(19) United States
(12) Patent Application Publication
(10) Pub. No.: US 2008/0039434 A1
(43) Pub. Date: Feb. 14, 2008

(54) Treatment of Interstitial Cystitis with Vitamin D Compounds

(75) Inventor: Enrico Colli, Milan (IT)

Correspondence Address: EDWARDS ANGELL PALMER & DODGE LLP, P.O. BOX 55874, BOSTON, MA, 02205, US

(73) Assignee: BioXell S. p.A., MILAN (IT)

(21) Appl. No.: 10/590,790

(22) PCT Filed: Mar. 1, 2005
(86) PCT No.: PCT/EP05/50902

§ 371 Date: May 15, 2007

(30) Foreign Application Priority Data
Mar. 1, 2004 (GB) GB 0404571.2
Mar. 1, 2004 (GB) GB 0404567.0

(51) Int. Cl. A61K 31/59; A61P 13/10

(52) U.S. Cl. 514/167

(57) ABSTRACT

There is provided according to the invention a vitamin D compound for use in the prevention and/or treatment of interstitial cystitis. Also provided is a method for preventing and/or treating interstitial cystitis by administering an effective amount of a vitamin D compound.

The present invention is concerned with the use of vitamin D compounds for the manufacture of a medicament for the prevention and/or treatment of interstitial cystitis. It is further concerned with a method for preventing and/or treating interstitial cystitis, by administering a vitamin D compound in an amount effective to prevent and/or to treat such disease alone or in combination with further agents.

Interstitial cystitis, referred to herein as “IC”, is a chronic inflammatory bladder disease, also known as chronic pelvic pain syndrome (CPPS) or painful bladder syndrome (PBS), characterized by pelvic pain, urinary urgency and frequency. This disease affects mainly females, although males are also diagnosed with IC. Unlike other bladder dysfunction conditions, IC is characterized by chronic inflammation of the bladder wall which is responsible for the symptomotology; in other words, the cause of the abnormal bladder contractility and chronic pelvic pain is the chronic inflammation and as a consequence the treatment should target this etiological component. In fact, the traditional treatment of bladder dysfunctions, like overactive bladder, with smooth muscle relaxant agents, is not effective in patients with IC.

Presently a large number of therapies are used for this disease, which reflects that this is a condition without a truly effective treatment. For example, intravesical dimethyl sulphoxide (DMSO) has been the subject of extensive clinical investigation. However, the mechanism of action is still unknown. The clinical results are not completely satisfactory and the route of administration (intravesical) is not ideal for the prolonged treatment often required in IC.

Some existing therapies are based on the concept of mucosal barrier protection, for example, use of the heparin analog pentosan polysulphate sodium (PPS). Again, the results are disappointing and on a long term basis, less than 20% of patients show a beneficial effect from the administration of oral PPS.

Other approaches include the use of antihistamines, flavonoids and other agents that may decrease the action of proinflammatory agents mediated by mast cells. Such approaches have shown inconsistent and marginal effectiveness in several studies. A further approach, the use of intravesical BCG (Bacille Calmette Guerin) also failed to show symptom improvement in a controlled cross-over trial versus DMSO.

As a consequence, there is a clear need to identify novel pharmacological approaches targeting all the different immunological factors involved in the etiology of the disease.

As described herein, it has now surprisingly been found that vitamin D analogues can treat and prevent interstitial cystitis.

The importance of vitamin D (cholecalciferol) in the biological systems of higher animals has been recognized since its discovery by Mellanby in 1920 (Mellanby, E. (1921) Spec. Rep. Ser. Med. Res. Council (GB) SRS 61:4). It was in the interval of 1920-1930 that vitamin D officially became classified as a “vitamin” that was essential for the normal development of the skeleton and maintenance of calcium and phosphorous homeostasis.

Studies involving the metabolism of vitamin D₃ were initiated with the discovery and chemical characterization of the plasma metabolite, 25-hydroxyvitamin D_{3 [}25(OH) D₃] (Blunt, J. W. et al. (1968) Biochemistry 6:3317-3322) and the hormonally active form, 1-alpha,25(OH)₂D₃ (Myrtle, J. F. et al. (1970) J. Biol. Chem. 245:1190-1196; Norman, A. W. et al. (1971) Science 173:51-54; Lawson, D. E. M. et al. (1971) Nature 230:228-230; Holick, M. F. (1971) Proc. Natl. Acad. Sci. USA 68:803-804). The formulation of the concept of a vitamin D endocrine system was dependent both upon appreciation of the key role of the kidney in producing 1-alpha,25(OH)₂D₃ in a carefully regulated fashion (Fraser, D. R. and Kodicek, E (1970) Nature 288:764-766; Wong, R. G. et al. (1972) J. Clin. Invest. 51:1287-1291), and the discovery of a nuclear receptor for 1-alpha,25(OH)₂D₃ (VD₃R) in the intestine (Haussler, M. R. et al. (1969) Exp. Cell Res. 58:234-242; Tsai, H. C. and Norman, A. W. (1972) J. Biol. Chem. 248:5967-5975).

The operation of the vitamin D endocrine system depends on the following: first, on the presence of cytochrome P450 enzymes in the liver (Bergman, T. and Postlind, H. (1991) Biochem. J. 276:427-432; Ohyama, Y and Okuda, K. (1991) J. Biol. Chem. 266:8690-8695) and kidney (Henry, H. L. and Norman, A. W. (1974) J. Biol. Chem. 249:7529-7535; Gray, R. W. and Ghazarian, J. G. (1989) Biochem. J. 259:561-568), and in a variety of other tissues to effect the conversion of vitamin D₃ into biologically active metabolites such as 1-alpha,25(OH)₂D₃ and 24R,25(OH)₂D₃; second, on the existence of the plasma vitamin D binding protein (DBP) to effect the selective transport and delivery of these hydrophobic molecules to the various tissue components of the vitamin D endocrine system (Van Baelen, H. et al. (1988) Ann NY Acad. Sci. 538:60-68; Cooke, N. E. and Haddad, J. G. (1989) Endocr. Rev. 10:294-307; Bikle, D. D. et al. (1986) J. Clin. Endocrinol. Metab. 63:954-959); and third, upon the existence of stereoselective receptors in a wide variety of target tissues that interact with the agonist 1-alpha,25(OH)₂D₃ to generate the requisite specific biological responses for this secosteroid hormone (Pike, J. W. (1991) Annu. Rev. Nutr. 11:189-216). To date, there is evidence that nuclear receptors for 1-alpha,25(OH)₂D₃ (VD₃R) exist in more than 30 tissues and cancer cell lines (Reichel, H. and Norman, A. W. (1989) Annu. Rev. Med. 40:71-78), including the normal bladder.

Vitamin D₃ and its hormonally active forms are well-known regulators of calcium and phosphorus homeostasis. These compounds are known to stimulate, at least one of, intestinal absorption of calcium and phosphate, mobilization of bone mineral, and retention of calcium in the kidneys. Furthermore, the discovery of the presence of specific vitamin D receptors in more than 30 tissues has led to the identification of vitamin D₃ as a pluripotent regulator outside its classical role in calcium/bone homeostasis. A paracrine role for 1-alpha,25(OH)₂ D₃ has been suggested by the combined presence of enzymes capable of oxidizing vitamin D₃ into its active forms, e.g., 25-OHD-1-alpha-hydroxylase, and specific receptors in several tissues such as bone, keratinocytes, placenta, and immune cells. Moreover, vitamin D₃ hormone and active metabolites have been found to be capable of regulating cell proliferation and differentiation of both normal and malignant cells (Reichel, H. et al. (1989) Ann. Rev. Med. 40: 71-78).

Given the activities of vitamin D₃ and its metabolites, much attention has focused on the development of synthetic analogues of these compounds. A large number of these analogues involve structural modifications in the A ring, B ring, C/D rings, and, primarily, the side chain (Bouillon, R. et al. (1995) Endocrine Reviews 16(2):201-204). Although a vast majority of the vitamin D₃ analogues developed to date involve structural modifications in the side chain, a few studies have reported the biological profile of A-ring diastereomers (Norman, A. W. et al. (1993) J. Biol. Chem. 268 (27): 20022-20030). Furthermore, biological esterification of steroids has been studied (Hochberg, R. B., (1998) Endocr Rev. 19(3): 331-348), and esters of vitamin D₃ are known (WO 97/11053).

Moreover, despite much effort in developing synthetic analogues, clinical applications of vitamin D and its structural analogues have been limited by the undesired side effects elicited by these compounds after administration to a subject for known indications/applications of vitamin D compounds.

The activated form of vitamin D, vitamin D3, and some of its analogues have been described as potent regulators of cell growth and differentiation. It has previously been found that vitamin D3 as well as an analogue (analogue V), inhibited BPH cell proliferation and counteracted the mitogenic activity of potent growth factors for BPH cells, such as keratinocyte growth factor (KGF) and insulin-like growth factor (IGF1). Moreover, the analogue induced bcl-2 protein expression, intracellular calcium mobilization, and apoptosis in both unstimulated and KGF-stimulated BPH cells.

Thus the invention provides vitamin D compounds, and new methods of treatment using such compounds, for the prevention or treatment of interstitial cystitis.

Before further description of the present invention, and in order that the invention may be more readily understood, certain terms are first defined and collected here for convenience.

By “interstitial cystitis” (IC) it is meant a chronic, inflammatory disorder of the bladder characterized by variable degrees of urinary urgency, frequency and bladder pain. As described herein, the Inventor has shown that vitamin D₃ analogues have applications in the treatment of both the inflammatory component of IC and the consequent bladder overactivity characterizing IC, which contribute to the symptoms of pain, urgency and frequency seen in IC patients. Some IC patients may experience pain as their main symptom with minimal frequency and urgency, whilst other patients may present with only frequency and urgency symptoms. IC patients may or may not experience the additional symptom of nocturia. Whilst pain is currently considered to be the most important characteristic symptom of IC, nocturia is not considered essential for the diagnosis of IC. It is also believed that patients with normal frequency but with pain and urgency can also have IC. This indicates that IC patients can present with a wide range of symptomatic combinations. IC should be suspected in all patients who present with urinary discomfort, suprapubic pressure or heaviness or burning micturition with or without pain, in the absence of bacterial infection. IC is currently diagnosed on the basis of clinical features. The recommended tests include urinalysis, urine culture, cytology, urodynamics and cystoscopy under anesthesia with bladder distension.

The term “administration” or “administering” includes routes of introducing the vitamin D compound(s) to a subject to perform their intended function. Examples of routes of administration which can be used include injection (subcutaneous, intravenous, parenterally, intraperitoneally, oral, inhalation, rectal, transdermal or via bladder instillation. The pharmaceutical preparations are, of course, given by forms suitable for each administration route. For example, these preparations are administered in tablets or capsule form, by injection, infusion, inhalation, lotion, ointment, suppository, etc. Oral administration is preferred. The injection can be bolus or can be continuous infusion. Depending on the route of administration, the vitamin D compound can be coated with or disposed in a selected material to protect it from natural conditions which may detrimentally effect its ability to perform its intended function. The vitamin D compound can be administered alone, or in conjunction with either another agent as described above, for example with a smooth muscle relaxant (such as alpha blockers or anti-muscarinic drugs) or with a pharmaceutically-acceptable carrier, or both. The vitamin D compound can be administered prior to the administration of the other agent, simultaneously with the agent, or after the administration of the agent. Furthermore, the vitamin D compound can also be administered in a pro-form which is converted into its active metabolite, or more active metabolite in vivo.

The term “effective amount” includes an amount effective, at dosages and for periods of time necessary, to achieve the desired result, i.e. sufficient to treat interstitial cystitis. An effective amount of vitamin D compound may vary according to factors such as the disease state, age and weight of the subject, and the ability of the vitamin D compound to elicit a desired response in the subject. Dosage regimens may be adjusted to provide the optimum therapeutic response. An effective amount is also one in which any toxic or detrimental effects (e.g., side effects) of the vitamin D compound are outweighed by the therapeutically beneficial effects.

A therapeutically effective amount of vitamin D compound (i.e., an effective dosage) may range from about 0.001 to 30 ug/kg body weight, preferably about 0.01 to 25 ug/kg body weight, more preferably about 0.1 to 20 ug/kg body weight, and even more preferably about 1 to 10 ug/kg, 2 to 9 ug/kg, 3 to 8 ug/kg, 4 to 7 ug/kg, or 5 to 6 ug/kg body weight. The skilled artisan will appreciate that certain factors may influence the dosage required to effectively treat a subject, including but not limited to the severity of the disease or disorder, previous treatments, the general health and/or age of the subject, and other diseases present. In addition, the dose administered will also depend on the particular Vitamin D compound used, the effective amount of each compounds can be determined by titration methods known in the art. Moreover, treatment of a subject with a therapeutically effective amount of a vitamin D compound can include a single treatment or, preferably, can include a series of treatments. In one example, a subject is treated with a vitamin D compound in the range of between about 0.1 to 20 ug/kg body weight, one time per day for a duration of six months or longer, for example for life depending on management of the symptoms and the evolution of the condition. Also, as with other chronic treatments an “on-off” or intermittent treatment regime can be considered. It will also be appreciated that the effective dosage of a vitamin D compound used for treatment may increase or decrease over the course of a particular treatment.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. Other examples of lower alkyl include sec-butyl, n-butyl and pentyl. In preferred embodiment, the term “lower alkyl” includes a straight chain alkyl having 4 or fewer carbon atoms in its backbone, e.g., C₁-C₄ alkyl.

Thus specific examples of alkyl include C1-6 alkyl or C1-4alkyl (such as methyl or ethyl). Specific examples of hydroxyalkyl include C1-6hydroxyalkyl or C1-4hydroalkyl (such as hydroxymethyl).

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The terms “alkenyl” and “alkynyl” refer to unsaturated aliphatic groups analogueous in length and possible substitution to the alkyls described above, but that contain at least one double or triple bond, respectively. For example, the invention contemplates cyano and propargyl groups.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

As used herein, the term “halogen” designates —F, —Cl, —Br or —I; the term “sulfhydryl” or “thiol” means —SH; the term “hydroxyl” means —OH.

The term “haloalkyl” is intended to include alkyl groups as defined above that are mono-, di- or polysubstituted by halogen, e.g., C1-6haloalkyl or C1-4haloalkyl such as fluoromethyl and trifluoromethyl.

The term “heteroatom” as used herein means an atom of any element other than carbon or hydrogen. Preferred heteroatoms are nitrogen, oxygen, sulfur and phosphorus.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “isomers” or “stereoisomers” refers to compounds which have identical chemical constitution, but differ with regard to the arrangement of the atoms or groups in space.

The terms “isolated” or “substantially purified” are used interchangeably herein and refer to vitamin D₃ compounds in a non-naturally occurring state. The compounds can be substantially free of cellular material or culture medium when naturally produced, or chemical precursors or other chemicals when chemically synthesized. In certain preferred embodiments, the terms “isolated” or “substantially purified” also refer to preparations of a chiral compound which substantially lack one of the enantiomers; i.e., enantiomerically enriched or non-racemic preparations of a molecule. Similarly, the terms “isolated epimers” or “isolated diastereomers” refer to preparations of chiral compounds which are substantially free of other stereochemical forms. For instance, isolated or substantially purified vitamin D₃ compounds include synthetic or natural preparations of a vitamin D₃ enriched for the stereoisomers having a substituent attached to the chiral carbon at position 3 of the A-ring in an alpha-configuration, and thus substantially lacking other isomers having a beta-configuration. Unless otherwise specified, such terms refer to vitamin D₃ compositions in which the ratio of alpha to beta forms is greater than 1:1 by weight. For instance, an isolated preparation of an a epimer means a preparation having greater than 50% by weight of the alpha-epimer relative to the beta stereoisomer, more preferably at least 75% by weight, and even more preferably at least 85% by weight. Of course the enrichment can be much greater than 85%, providing “substantially epimer-enriched” preparations, i.e., preparations of a compound which have greater than 90% of the alpha-epimer relative to the beta stereoisomer, and even more preferably greater than 95%. The term “substantially free of the beta stereoisomer” will be understood to have similar purity ranges.

As used herein, the term “vitamin D compound” includes any compound being an analogue of vitamin D that is capable of treating or preventing interstitial cystitis. Generally, compounds which are ligands for the Vitamin D receptor (VDR ligands) and which are capable of treating or preventing interstitial cystitis are considered to be within the scope of the invention. Vitamin D compounds are preferably agonists of the vitamin D receptor. Thus, vitamin D compounds are intended to include secosteroids. Examples of specific vitamin D compounds suitable for use in the methods of the present invention are further described herein. A vitamin D compound includes vitamin D₂ compounds, vitamin D₃ compounds, isomers thereof, or derivatives/analogues thereof. Preferred vitamin D compounds are vitamin D3 compounds which are ligands of (more preferably are agonists of) the vitamin D receptor. Preferably the vitamin D compound (e.g., the vitamin D3 compound) is a more potent agonist of the vitamin D receptor than the native ligand (i.e. the vitamin D, e.g., vitamin D3). Vitamin D1 compounds, vitamin D2 compounds and vitamin D3 compounds include, respectively, vitamin D1, D2, D3 and analogues thereof. In certain embodiments, the vitamin D compound may be a steroid, such as a secosteroid, e.g., calciol, calcidiol or calcitriol. Non-limiting examples of vitamin D compounds in accordance with the invention include those described in U.S. Pat. Nos. 6,017,908, 6,100,294, 6,030,962, 5,428,029 and 6,121,312, published international applications WO 98/51633, WO 01/40177A3.

The term “secosteroid” is art-recognized and includes compounds in which one of the cyclopentanoperhydro-phenanthrene rings of the steroid ring structure is broken. For example, 1-alpha,25(OH)₂D₃ and analogues thereof are hormonally active secosteroids. In the case of vitamin D₃, the 9-10 carbon-carbon bond of the B-ring is broken, generating a seco-B-steroid. The official IUPAC name for vitamin D₃ is 9,10-secocholesta-5,7,10(19)-trien-3B-ol. For convenience, a 6-s-trans conformer of 1alpha,25(OH)₂D₃ is illustrated herein having all carbon atoms numbered using standard steroid notation.

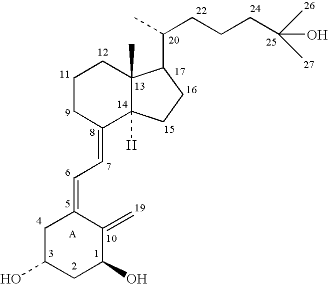

In the formulas presented herein, the various substituents on ring A are illustrated as joined to the steroid nucleus by one of these notations: a dotted line (----) indicating a substituent which is in the beta-orientation (i.e., above the plane of the ring), a wedged solid line [symbol] indicating a substituent which is in the alpha-orientation (i.e., below the plane of the molecule), or a wavy line [symbol] indicating that a substituent may be either above or below the plane of the ring. In regard to ring A, it should be understood that the stereochemical convention in the vitamin D field is opposite from the general chemical field, wherein a dotted line indicates a substituent on Ring A which is in an alpha-orientation (i.e., below the plane of the molecule), and a wedged solid line indicates a substituent on ring A which is in the beta-orientation (i.e., above the plane of the ring).

Furthermore the indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. Regardless, both configurations, cis/trans and/or Z/E are contemplated for the compounds for use in the present invention.

As shown, the A ring of the hormone 1-alpha,25(OH)₂D₃ contains two asymmetric centers at carbons 1 and 3, each one containing a hydroxyl group in well-characterized configurations, namely the 1-alpha- and 3-beta-hydroxyl groups. In other words, carbons 1 and 3 of the A ring are said to be “chiral carbons” or “carbon centers.”

With respect to the nomenclature of a chiral center, terms “d” and “l” configuration are as defined by the IUPAC Recommendations. As to the use of the terms, diastereomer, racemate, epimer and enantiomer will be used in their normal context to describe the stereochemistry of preparations.

Also, throughout the patent literature, the A ring of a vitamin D compound is often depicted in generic formulae as any one of the following structures: 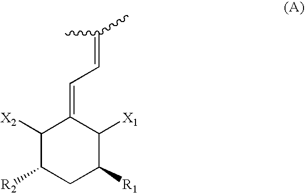
wherein X₁ and X₂ are defined as H or ═CH₂; or 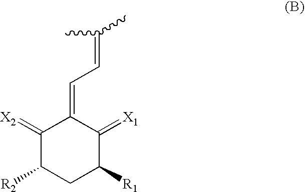
wherein X₁ and X₂ are defined as H₂ or CH₂.

Although there does not appear to be any set convention, it is clear that one of ordinary skill in the art understands either formula (A) or (B) to represent an A ring in which, for example, X₁ is ═CH₂ and X₂ is defined as H₂, as follows: 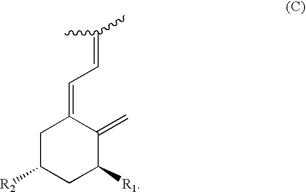
For purposes of the instant invention, formula (B) will be used in all generic structures.

Thus, in one aspect, the invention provides the use of a Vitamin D compound in the prevention or treatment of interstitial cystitis. Also provided is a method of treating a patient with interstitial cystitis by administering an effective amount of a Vitamin D compound. Further provided is the use of a Vitamin D compound in the manufacture of a medicament for the prevention or treatment of interstitial cystitis. Further provided is a vitamin D compound for use in the prevention and/or treatment of interstitial cystitis. Also provided is a kit containing a vitamin D compound together with instructions directing administration of said compound to a patient in need of treatment and/or prevention of interstitial cystitis thereby to treat and/or prevent interstitial cystitis in said patient. Interstitial cystitis may, for example, be characterized by the presence of symptoms of bladder dysfunction and bladder inflammation.

The methods and uses of the invention may, in one embodiment of the invention, be methods and uses in treating females. In another embodiment they are methods and uses in treating males.

In one embodiment of the invention, the vitamin D compound is a compound of formula (I): 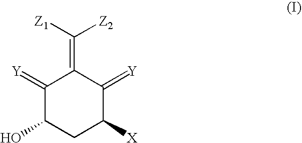
wherein:
X is hydroxyl or fluoro;
Y is H₂ or CH₂;
Z₁ and Z₂ are H or a substituent represented by formula (II), provided Z₁ and Z₂ are different (preferably Z₁ and Z₂ do not both represent formula (II)): 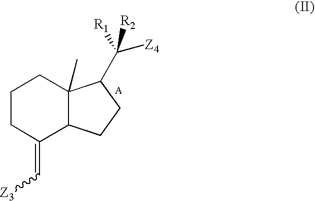
   wherein:
Z₃ represents the above-described formula (I);
A is a single bond or a double bond;
R₁, R₂, and Z₄, are each, independently, hydrogen, alkyl, or a saturated or unsaturated carbon chain represented by formula (III), provided that at least one of R₁, R₂, and Z₄ is the saturated or unsaturated carbon chain represented by formula (III) and provided that all of R₁, R₂, and Z₄ are not saturated or unsaturated carbon chain represented by formula (III): 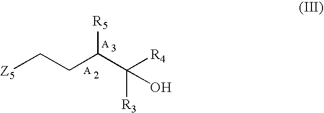
   wherein:
Z₅ represents the above-described formula (II);
A₂ is a single bond, a double bond, or a triple bond; and
A₃ is a single bond or a double bond; and
R₃, and R₄, are each, independently, hydrogen, alkyl, haloalkyl, hydroxyalkyl; and
R₅ is H₂ or oxygen. R₅ may also represent hydrogen or may be absent.

Thus, in the above structure of formula (III) (and in corresponding structures below), when A₂ represents a triple bond R₅ is absent. When A₂ represents a double bond R₅ represents hydrogen. When A₂ represents a single bond R₅ represents a carbonyl group or two hydrogen atoms.

In another embodiment of the invention, the vitamin D compound is a compound of formula (IV): 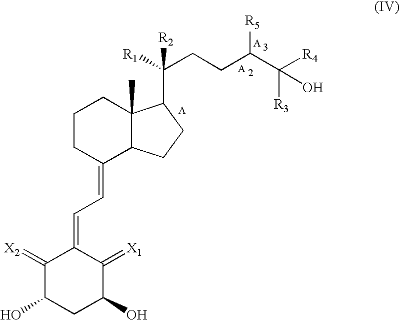
wherein:
X₁ and X₂ are H₂ or CH₂, wherein X₁ and X₂ are not CH₂ at the same time;
A is a single or double bond;
A₂ is a single, double or triple bond;
A₃ is a single or double bond;
R₁ and R₂ are hydrogen, C₁-C₄ alkyl or 4-hydroxy-4-methylpentyl, wherein R₁ and
R₂ are not both hydrogen;
R₅ is H₂ or oxygen, R₅ may also represent hydrogen or may be absent;
R₃ is C₁-C₄ alkyl, hydroxyalkyl or haloalkyl, eg., fluoroalkyl, e.g., fluoromethyl and trifluoromethyl; and
R₄ is C₁-C₄ alkyl, hydroxyalkyl or haloalkyl, eg., fluoroalkyl, e.g., fluoromethyl and trifluoromethyl.

In yet another embodiment of the invention, the vitamin D compound is a compound of formula (V): 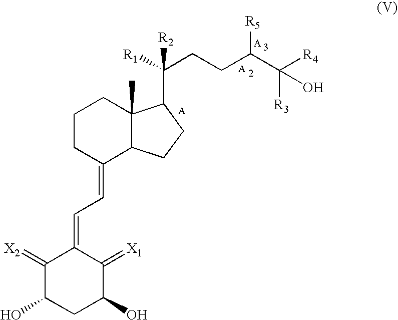
wherein:
X₁ and X₂ are H₂ or CH₂, wherein X₁ and X₂ are not CH₂ at the same time;
A is a single or double bond;
A₂ is a single, double or triple bond;
A₃ is a single or double bond;
R₁ and R₂ are hydrogen, C₁-C₄ alkyl, wherein R₁ and R₂ are not both hydrogen;
R₅ is H₂ or oxygen, R₅ may also represent hydrogen or may be absent;
R₃ is C₁-C₄ alkyl, hydroxyalkyl or haloalkyl, e.g., fluoroalkyl, e.g., fluoromethyl and trifluoromethyl; and
R₄ is C₁-C₄ alkyl, hydroxyalkyl haloalkyl, e.g., or fluoroalkyl, e.g., fluoromethyl and trifluoromethyl.
   An example of the above structure of formula (V) is 1,25-dihydroxy-16-ene-23-yne cholecalciferol.

In yet another embodiment, the vitamin D compound is a “geminal” compound of formula (VI): 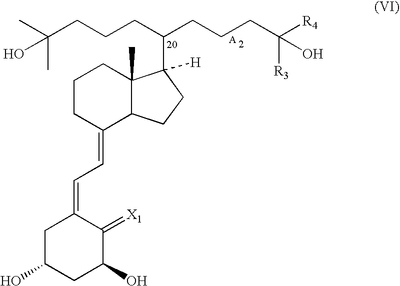
wherein:
X₁ is H₂ or CH₂;
A₂ is a single, a double or a triple bond;
R₃ is C₁-C₄ alkyl, hydroxyalkyl, or haloalkyl, e.g., fluoroalkyl, e.g., fluoromethyl and trifluoromethyl;
R₄ is C₁-C₄ alkyl, hydroxyalkyl or haloalkyl, e.g., fluoroalkyl, e.g., fluoromethyl and trifluoromethyl;
and the configuration at C₂₀ is R or S.

Compounds of this type may be referred to as “geminal” or “gemini” vitamin D₃ compounds due to the presence of two alkyl chains at C20.

An example geminal compound of formula (VI) is 1,25-dihydroxy-21-(3-hydroxy-3-methylbutyl)-19-nor-cholecalciferol hereinafter referred to as “Compound I”:

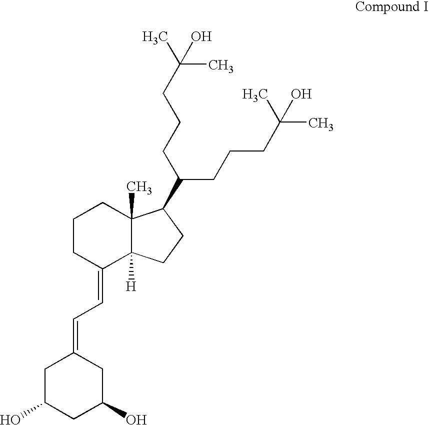

The synthesis of Compound I is described in WO98/49138 and U.S. Pat. No. 6,030,962 which are herein incorporated in itheir entirety by reference.

In another embodiment, the vitamin D compound is a compound of formula (VII): 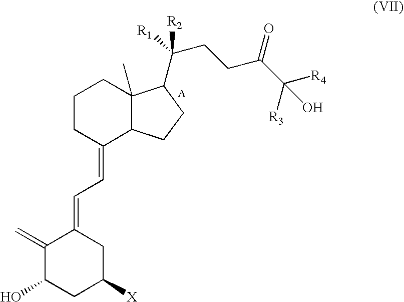
wherein:

A is a single or double bond;

R₁ and R₂ are each, independently, hydrogen, alkyl (for example methyl);

R₃, and R₄, are each, independently, alkyl, and

X is hydroxyl or fluoro.
In a further embodiment, the vitamin D compound is a compound having formula (VIII): 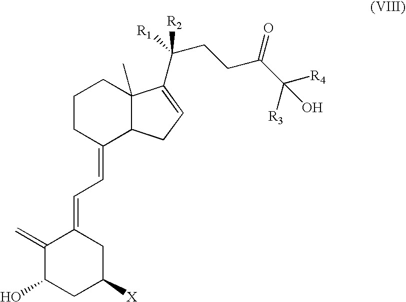
wherein:

R₁ and R₂, are each, independently, hydrogen, or alkyl, e.g., methyl;

R₃ is alkyl, e.g., methyl,

R₄ is alkyl, e.g., methyl; and

X is hydroxyl or fluoro.

In specific embodiments of the invention, the vitamin D compound is selected from the group consisting of:

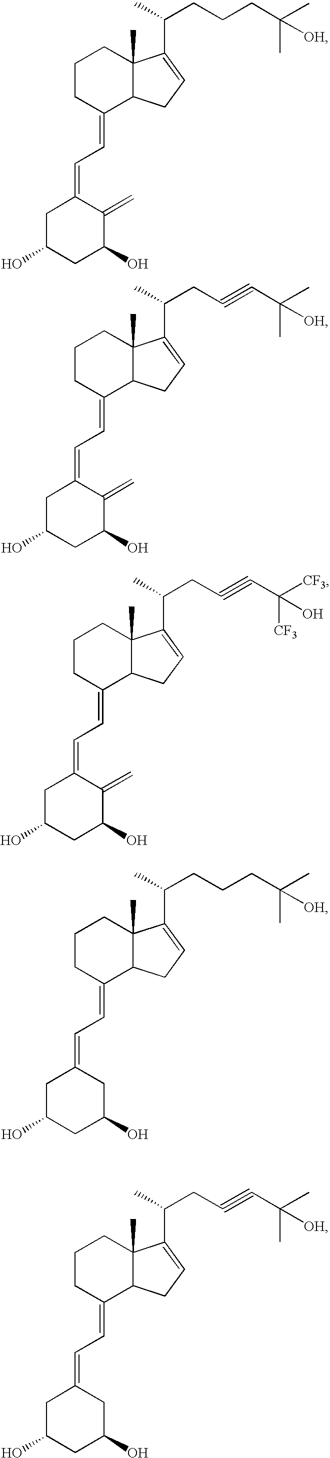

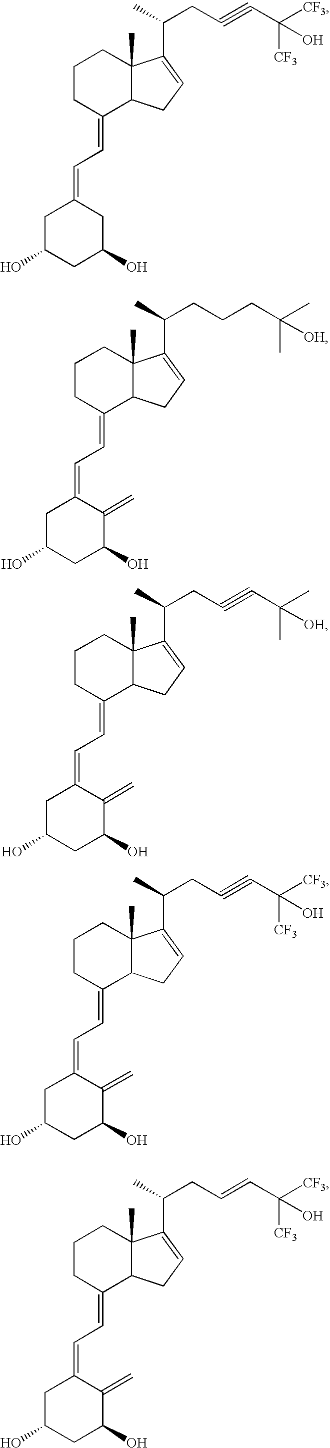

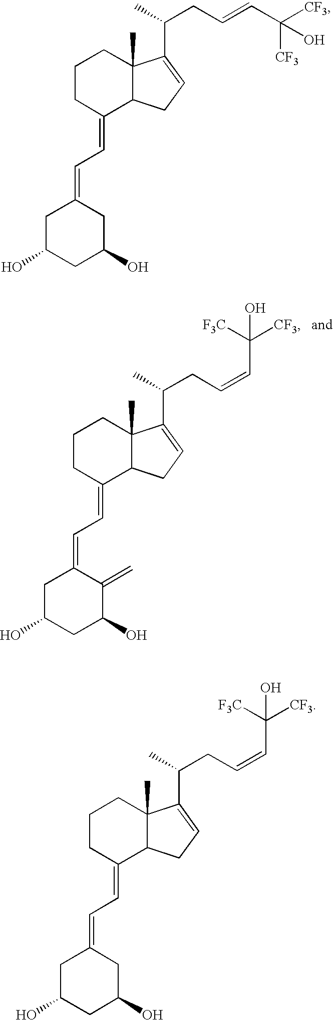

In other specific embodiments of the invention, the vitamin D compound is selected from the group consisting of:

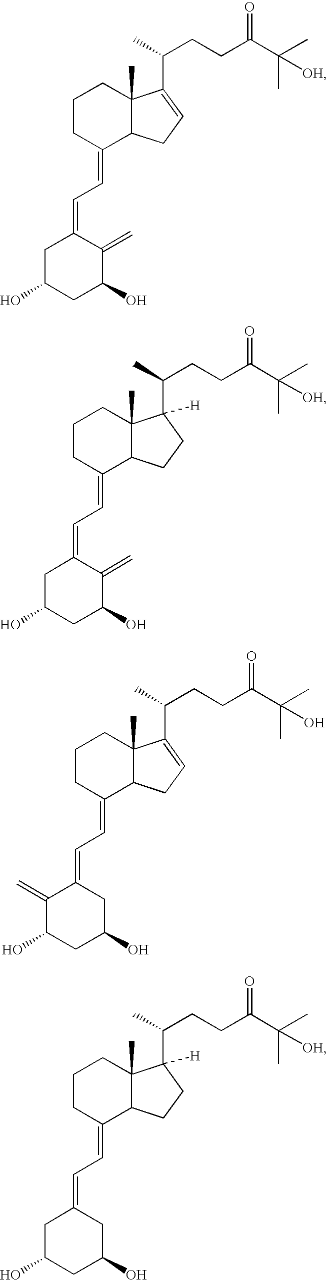

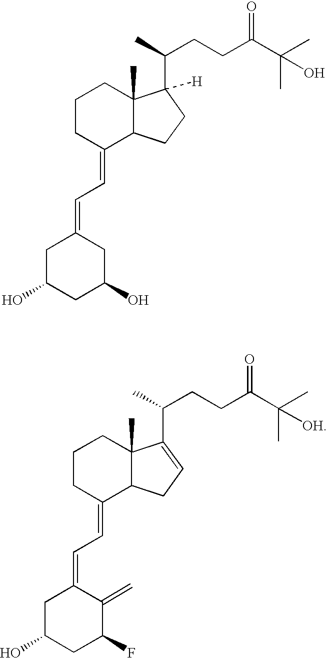

In further specific embodiments of the invention, the vitamin D compound is selected from the group of geminal compounds consisting of:

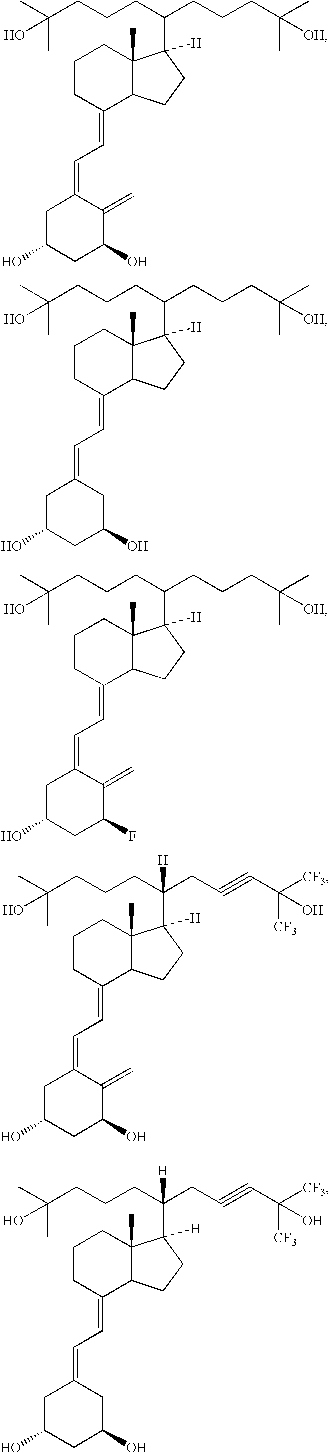

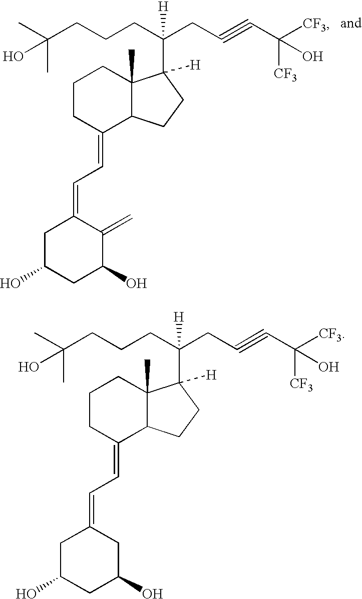

In still further specific embodiments of the invention, the vitamin D compound is a geminal compound of formula (IX): 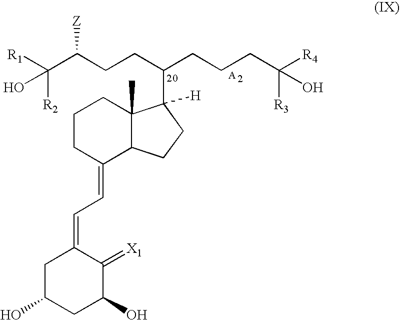
wherein:

X₁ is H₂ or CH₂;

A₂ is a single, a double or a triple bond;

R₁, R₂, R₃ and R₄ are each independently C₁-C₄ alkyl, hydroxyalkyl, or haloalkyl, e.g., fluoroalkyl, e.g., fluoromethyl and trifluoromethyl;

Z is —OH, Z may also be ═O, —NH₂ or —SH; and

the configuration at C₂₀ is R or S,

and pharmaceutically acceptable esters, salts, and prodrugs thereof.

In a further embodiment, X₁ is CH₂. In another embodiment, A₂ is a single bond. In another, R₁, R₂, R₃, and R₄ are each independently methyl or ethyl. In a further embodiment, Z is —OH. In another, X₁ is CH₂; A₂ is a single bond; R₁, R₂, R₃, and R₄ are each independently methyl or ethyl; and Z is —OH. In an even further embodiment, R₁, R₂, R₃, and R₄ are each methyl.

In a further embodiment of the invention, the vitamin D compound is a geminal compound of the formula: 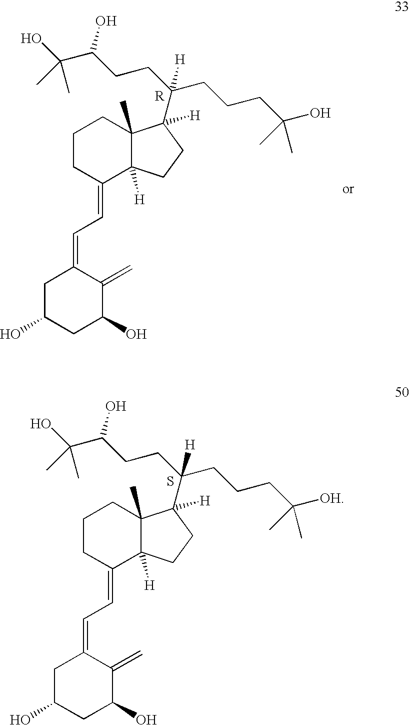
The chemical names of compounds 2 and 3 mentioned above are 1,25-dihydroxy-21-(2R,3-dihydroxy-3-methyl-butyl)-20R-cholecalciferol and 1,25-dihydroxy-21-(2R,3-dihydroxy-3-methyl-butyl)-20S-cholecalciferol respectively.

Additional embodiments of geminal compounds include the following vitamin D compounds for use in accordance with the invention: 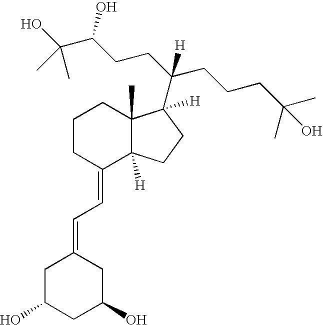
(1,25-Dihydroxy-21-(2R,3-dihydroxy-3-methyl-butyl)-20S-19-nor-cholecalciferol), 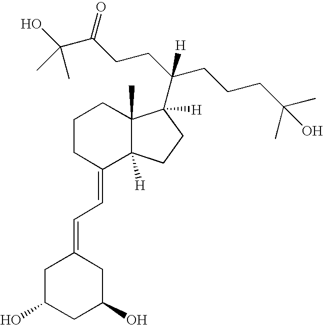
(1,25-Dihydroxy-20S-21-(3-hydroxy-3-methyl-butyl)-24-keto-19-nor-cholecalciferol), 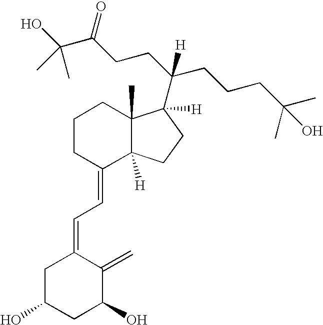
(1,25-Dihydroxy-20S-21-(3-hydroxy-3-methyl-butyl)-24-keto-cholecalciferol), 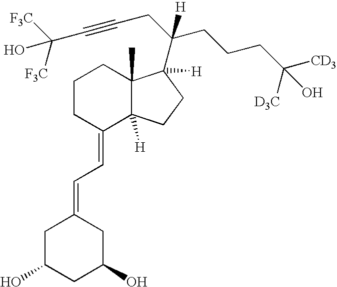
(1,25-Dihydroxy-21-(3-hydroxy-3-trifluoromethyl-4-trifluoro-butynyl)-26,27-hexadeutero-19-nor-20S-cholecalciferol)
   and 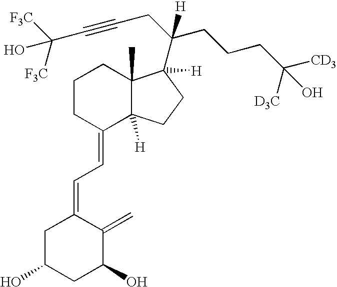
(1,25-Dihydroxy-21 (3-hydroxy-3-trifluoromethyl-4-trifluoro-butynyl)-26,27-hexadeutero-20S-cholecalciferol).

In further embodiments of the invention, the vitamin D compound is a compound of formula (X): 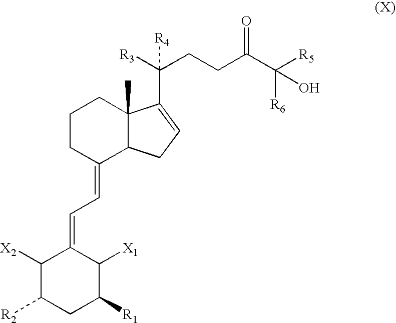
wherein:
X₁ and X₁ are each independently H₂ or ═CH₂, provided X₁ and X₁ are not both ═CH₂;
R₁ and R₂ are each independently, hydroxyl, OC(O)C₁-C₄ alkyl, OC(O)hydroxyalkyl, OC(O)fluororalkyl;
R₃ and R₄ are each independently hydrogen, C₁-C₄ alkyl hydroxyalkyl or haloalkyl, or R₃ and R₄ taken together with C₂₀ form C₃-C₆ cylcoalkyl; and
R₅ and R₆ are each independently C₁-C₄ alkyl
   and pharmaceutically acceptable esters, salts, and prodrugs thereof.

Suitably R₃ and R₄ are each independently hydrogen, C₁-C₄ alkyl, or R₃ and R₄ taken together with C₂₀ form C₃-C₆ cylcoalkyl.

In one example set of compounds R₅ and R₆ are each independently C₁-C₄ alkyl.

In another example set of compounds R₅ and R₆ are each independently haloalkyl e.g., C₁-C₄ fluoroalkyl.

When R₃ and R₄ are taken together with C20 to form C₃-C₆ cycloalkyl, an example is cyclopropyl.

In one embodiment, X₁ and X₁ are each H₂. In another embodiment, R₃ is hydrogen and R₄ is C₁-C₄ alkyl. In a preferred embodiment R₄ is methyl.

In another embodiment, R₅ and R₆ are each independently methyl, ethyl fluoromethyl or trifluoromethyl. In a preferred embodiment, R₅ and R₆ are each methyl.

In yet another embodiment, R₁ and R₁ are each independently hydroxyl or OC(O)C₁-C₄ alkyl. In a preferred embodiment, R₁ and R₁ are each OC(O)C₁-C₄ alkyl. In another preferred embodiment, R₁ and R₁ are each acetyloxy.

An example of such a compound is 1,3-O-diacetyl-1,25-dihydroxy-16-ene-24-keto-19-nor-cholecalciferol, having the following structure:

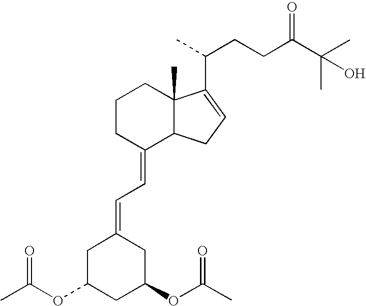

In another embodiment of the invention the vitamin D compound for use in accordance with the invention is 2-methylene-19-nor-20(S)-1-alpha,25-hydroxyvitamin D3:

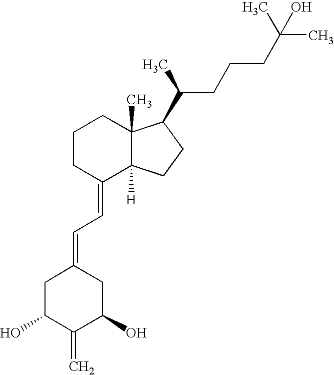

The synthesis of this and related compounds is described in WO02/05823 and U.S. Pat. No. 5,536,713 which are herein incorporated in their entirety by reference.

In another embodiment of the invention, the vitamin D compound is a compound of the formula (XII): 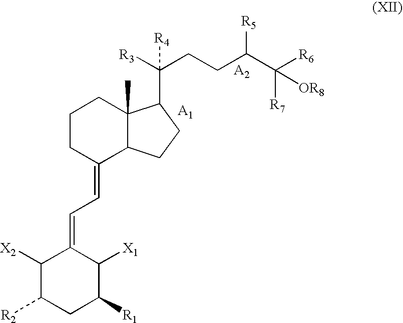
wherein:
A is single or double bond;
A₂ is a single, double or triple bond;
X₁ and X₂ are each independently H or ═CH₂, provided X₁ and X₂ are not both ═CH₂;
R₁ and R₂ are each independently OC(O)C₁-C₄ alkyl (for example OAc), OC(O)hydroxyalkyl, OROC(O)haloalkyl;
R₃, R₄ and R₅ are each independently hydrogen, C₁-C₄ alkyl, hydroxyalkyl, or haloalkyl, or R₃ and R₄ taken together with C₂₀ form C₃-C₆ cycloalkyl; and
R₆ and R₇ are each independently C₁₋₄alkyl or haloalkyl; and
R₈ is H, —COC₁-C₄alkyl (e.g. Ac), —COhydroxyalkyl or —COhaloalkyl; and
   pharmaceutically acceptable esters, salts, and prodrugs thereof.

When R₃ and R₄ are taken together with C₂₀ to form C₃-C₆ cycloalkyl an example is cyclopropyl.

Suitably R₆ and R₇ are each independently haloalkyl. R₈ may suitably represent H or Ac.

In one embodiment, A₁ is a single bond and A₂ is a single bond, E or Z double bond, or a triple bond. In another embodiment, A₁ is a double bond and A₂ is a single bond, E or Z double bond, or a triple bond. One of ordinary skill in the art will readily appreciate that when A₂ is a triple bond, R₅ is absent

In one embodiment, X₁ and X₂ are each H. In another embodiment, X₁ is CH₂ and X₂ is H₂. In another embodiment, R₃ is hydrogen and R₄ is C₁-C₄ alkyl. In a preferred embodiment R₄ is methyl.

In another example set of compounds R₁ and R₂ both represent OAc.

In one set of example compounds R₆ and R₇ are each independently C₁₋₄alkyl.

In another set of example compounds R₆ and R₇ are each independently haloalkyl. In another embodiment, R₆ and R₇ are each independently methyl, ethyl or fluoroalkyl. In a preferred embodiment, R₆ and R₈ are each trifluoroalkyl, e.g., trifluoromethyl.

Suitably R₅ represents hydrogen.

Thus, in certain embodiments, vitamin D compounds for use in accordance with the invention are represented by formula (XII): 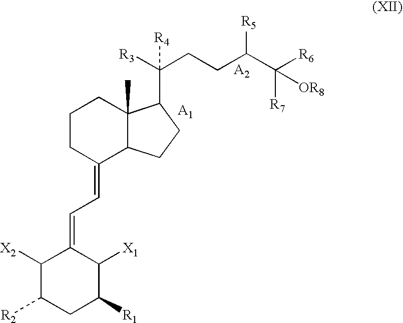
wherein:

A₁ is single or double bond;

A₂ is a single, double or triple bond;

X₁ and X₂ are each independently H or ═CH₂, provided X₁ and X₂ are not both ═CH₂;

R₁ and R₂ are each independently OC(O)C₁-C₄ alkyl, OC(O)hydroxyalkyl, or OC(O)haloalkyl;

R₃, R₄ and R₅ are each independently hydrogen, C₁-C₄ alkyl, hydroxyalkyl, or haloalkyl, or R₃ and R₄ taken together with C₂₀ form C₃-C₆ cycloalkyl;

R₆ and R₇ are each independently haloalkyl; and

R₈ is H, C(O)C₁-C₄ alkyl, C(O)hydroxyalkyl, or C(O)haloalkyl; and

pharmaceutically acceptable esters, salts, and prodrugs thereof.

An example compound of the above-described formula (XII) which is particularly preferred in the context of the present invention is 1,3-di-O-acetyl-1,25-dihydroxy-16,23Z-diene-26,27-hexafluoro-19-nor-cholecalciferol (“Compound A”):

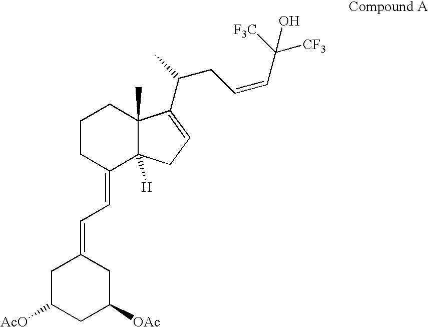

In another preferred embodiment the compound is one of formula (XIII), wherein R₁ and R₂ are each OAc; A₁ is a double bond; A₂ is a triple bond; and R₈ is either H or Ac:

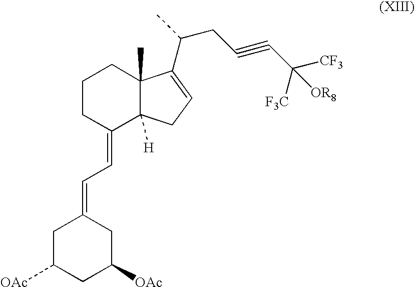

In certain embodiments of the above-represented formula (XII), vitamin D compounds for use in accordance with the invention are represented by the formula (XIV): 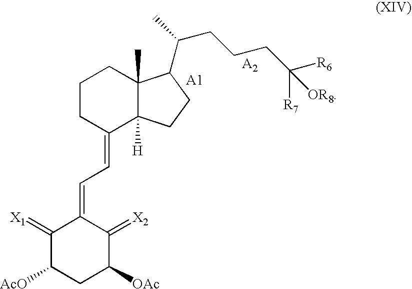
Other example compounds of the above-described formula (XIV) include:
1,3-di-O-acetyl-1,25-dihydroxy-23-yne-cholecalciferol;
1,3-di-O-acetyl-1,25-dihydroxy-16-ene-23-yne-cholecalciferol;
1,3-di-O-acetyl-1,25-dihydroxy-16,23E-diene-cholecalciferol;
1,3-di-O-acetyl-1,25-dihydroxy-16-ene-cholecalciferol;
1,3,25-Tri-O-acetyl-1,25-dihydroxy-16-ene-23-yne-26,27-hexafluoro-cholecalciferol:
1,3-di-O-acetyl-1,25-dihydroxy-16-ene-23-yne-26,27-hexafluoro-cholecalciferol;
1,3-Di-O-acetyl-1,25-dihydroxy-16,23E-diene-25R-26-trifluoro-cholecalciferol;
1,3-Di-O-acetyl-1,25-Dihydroxy-16-ene-23-yne-26,27-hexafluoro-19-nor-cholecalciferol;
1,3,25-Tri-O-acetyl-1,25-Dihydroxy-16-ene-23-yne-26,27-hexafluoro-19-nor-cholecalciferol;
1,3-di-O-acetyl-1,25-dihydroxy-16-ene-19-nor-cholecalciferol (“Compound C”);
1,3-Di-O-acetyl-1,25-dihydroxy-16-ene-23-yne-19-nor-cholecalciferol;
1,3-Di-O-acetyl-1,25-dihydroxy-16-ene-23-yne-26,27-bishomo-19-nor-cholecalciferol;

In certain other embodiments of the above-represented formula (XII), the vitamin D compounds for use in accordance with the invention are represented by the formula (XV):

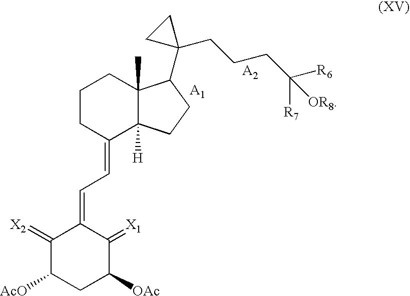

Other example compounds of the above-described formula (XV) include:
1,3-Di-O-acetyl-1,25-dihydroxy-20-cyclopropyl-23-yne-19-nor-cholecalciferol:
1,3,25-tri-O-acetyl-1,25-dihydroxy-20-cyclopropyl-23-yne-26,27-hexafluoro-19-nor-cholecalciferol;
1,3-di-O-acetyl-1,25-dihydroxy-20-cyclopropyl-23-yne-26,27-hexafluoro-19-nor-cholecalciferol;
1,3-di-O-acetyl-1,25-dihydroxy-20-cyclopropyl-23-yne-cholecalciferol;
1,3-di-O-acetyl-1,25-dihydroxy-20-cyclopropyl-23Z-ene-26,27-hexafluoro-19-nor-cholecalciferol;
1,3-di-O-acetyl-1,25-dihydroxy-20-cyclopropyl-cholecalciferol (“Compound F”);
1,3-di-O-acetyl-1,25-dihydroxy-16-ene-20-cyclopropyl-19-nor-cholecalciferol; and
1,3-Di-O-acetyl-1,25-dihydroxy-16-ene-20-cyclopropyl-cholecalciferol.

A preferred compound of formula XV is 1,3-di-O-acetyl-1,25-dihydroxy-20-cyclopropyl-23E-ene-26,27-hexafluoro-19-nor-cholecalciferol (“Compound E”):

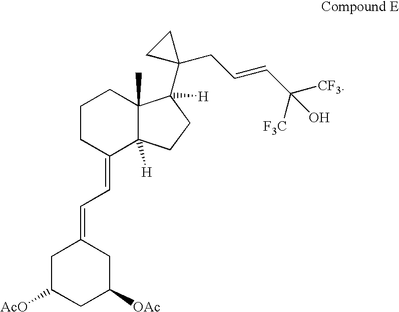

In a further embodiment, vitamin D compounds for use in the invention are compounds of the formula (XVI): 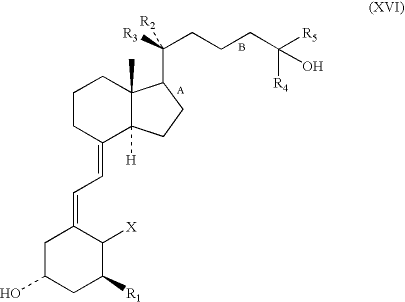
wherein:

X is H₂ or CH₂

R₁ is hydrogen, hydroxy or fluorine

R₂ is hydrogen or methyl

R₃ is hydrogen or methyl. When R₂ or R₃ is methyl, R₃ or R₂ must be hydrogen.

R₄ is methyl, ethyl or trifluoromethyl

R₅ is methyl, ethyl or trifluoromethyl

A is a single or double bond

B is a single, E-double, Z-double or triple bond.

In preferred compounds, each of R₄ and R₅ is methyl or ethyl, for example 1-alpha-fluoro-25-hydroxy-16,23E-diene-26,27-bishomo-20-epi-cholecalciferol (referred to as “Compound B” in examples, having the formula:

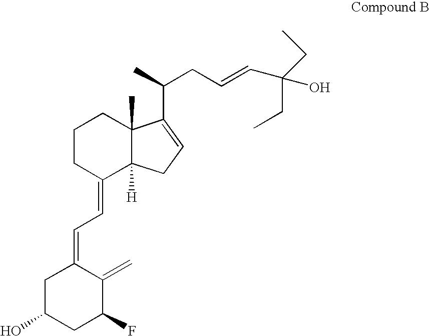

Such compounds are described in U.S. Pat. No. 5,939,408 and EP808833, the contents of which are herein incorporated by reference in their entirety. The invention also embraces use of esters and salts of Compound B. Esters include pharmaceutically acceptable labile esters that may be hydrolysed in the body to release Compound B. Salts of Compound B include adducts and complexes that may be formed with alkali and alkaline earth metal ions and metal ion salts such as sodium, potassium and calcium ions and salts thereof such as calcium chloride, calcium malonate and the like. However, although Compound B may be administered as a pharmaceutically acceptable salt or ester thereof, preferably Compound B is employed as is i.e., it is not employed as an ester or a salt thereof.

Other preferred vitamin D compounds for use in accordance with the invention included those having formula (XVII): 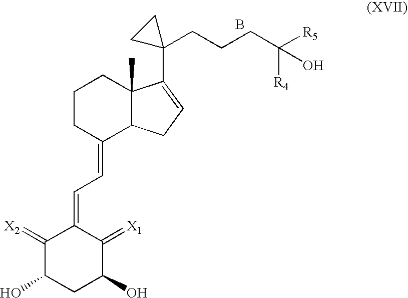
wherein:
B is single, double, or triple bond;
X₁ and X₂ are each independently H₂ or CH₂, provided X₁ and X₂ are not both CH₂; and
R₄ and R₅ are each independently alkyl or haloalkyl.

Compounds of formula (XVII) including the following:

### 1,25-Dihydroxy-16-ene-23-yne-20-cyclopyl-cholecalciferol

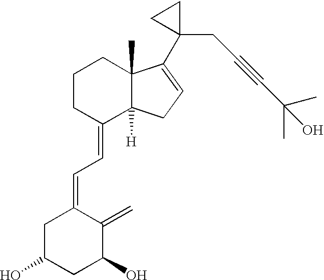

### 1,25-Dihydroxy-16-ene-23-yne-20-cyclopropyl-19-nor-cholecalciferol

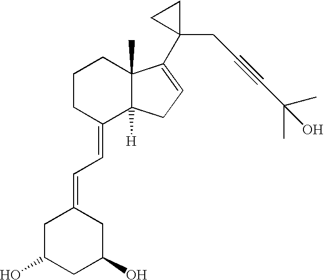

### 1,25-Dihydroxy-16-ene-20-cyclopropyl-23-yne-26,27-hexafluoro-19-nor-cholecalciferol

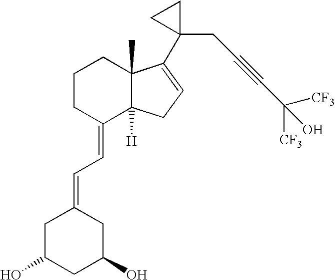

### 1,25-Dihydroxy-16-ene-20-cyclopropyl-23-yne-26,27-hexafluoro-cholecalciferol

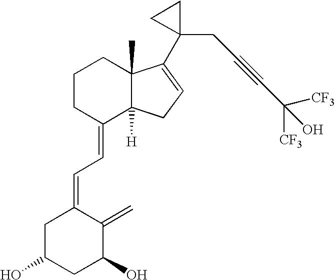

### 1,25-Dihydroxy-16,23E-diene-20-cyclopropyl-26,27-hexafluoro-19-nor-cholecalciferol

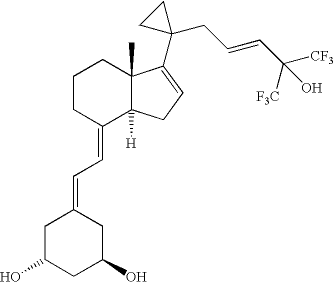

### 1,25-Dihydroxy-16,23E-diene-20-cyclopropyl-26,27-hexafluoro-cholecalciferol

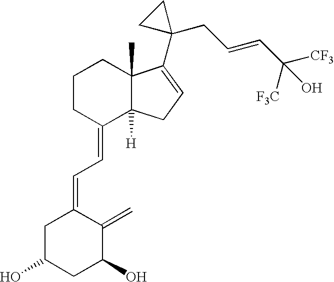

### 1,25-Dihydroxy-16,23Z-diene-20-cyclopropyl-26,27-hexafluoro-19-nor-cholecalciferol

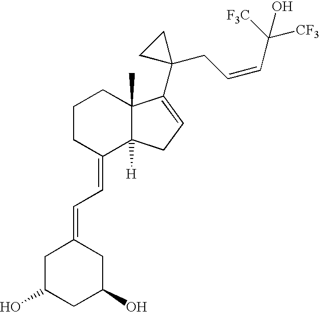

### 1,25-Dihydroxy-16,23Z-diene-20-cyclopropyl-26,27-hexafluoro-cholecalciferol

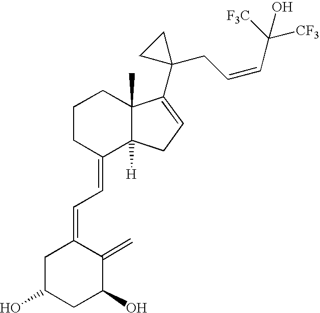

### 1,25-Dihydroxy-16-ene-20-cyclopropyl-19-nor-cholecalciferol

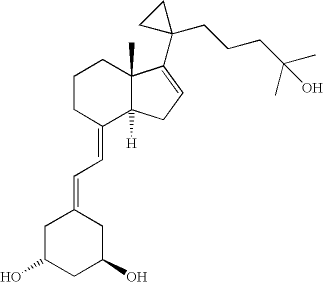

### 1,25-Dihydroxy-16-ene-20-cyclopropyl-cholecalciferol (“Compound H”)

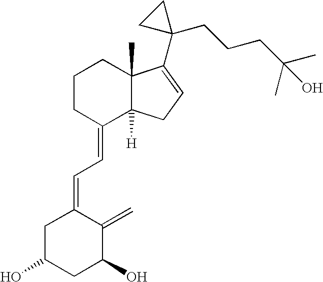

Another vitamin D compound of the invention is 1,25-dihydroxy-21(3-hydroxy-3-trifluoromethyl-4-trifluoro-butynyl)-26,27-hexadeutero-19-nor-20S-cholecalciferol.

The use of compounds having the structures given above is extended to pharmaceutically acceptable esters, salts, and prodrugs thereof.

A vitamin D compound of particular interest is calcitriol.

Other example compounds of use in the invention which are vitamin D receptor agonists include paricalcitol (ZEMPLAR™) (see U.S. Pat. No. 5,587,497), tacalcitol (BONALFA™) (see U.S. Pat. No. 4,022,891), doxercalciferol (HECTOROL™) (see Lam et al. (1974) Science 186,1038), maxacalcitol (OXAROL™) (see U.S. Pat. No. 4,891,364), calcipotriol (DAIVONEX™) (see U.S. Pat. No. 4,866,048), and falecalcitriol (FULSTAN™).

Other compounds include ecalcidene, calcithiazol and tisocalcitate.

Other compounds include atocalcitol, lexacalcitol and seocalcitol.

Another compound of possible interest is secalciferol (“OSTEO D”).

Other non-limiting examples of vitamin D compounds that may be of use in accordance with the invention include those described in published international applications: WO 01/40177, WO0010548, WO0061776, WO0064869, WO0064870, WO0066548, WO0104089, WO0116099, WO0130751, WO0140177, WO0151464, WO0156982, WO0162723, WO0174765, WO0174766, WO0179166, WO0190061, WO0192221, WO0196293, WO02066424, WO0212182, WO0214268, WO03004036, WO03027065, WO03055854, WO03088977, WO04037781, WO04067504, WO8000339, WO8500819, WO8505622, WO8602078, WO8604333, WO8700834, WO8910351, WO9009991, WO9009992, WO9010620, WO9100271, WO9100855, WO9109841, WO9112239, WO9112240, WO9115475, WO9203414, WO9309093, WO9319044, WO9401398, WO9407851, WO9407852, WO9408958, WO9410139, WO9414766, WO9502577, WO9503273, WO9512575, WO9527697, WO9616035, WO9616036, WO9622973, WO9711053, WO9720811, WO9737972, WO9746522, WO9818759, WO9824762, WO9828266, WO9841500, WO9841501, WO9849138, WO9851663, WO9851664, WO9851678, WO9903829, WO9912894, WO9915499, WO9918070, WO9943645, WO9952863, those described in U.S. patent Nos.: U.S. Pat. No. 3,856,780, U.S. Pat. No. 3,994,878, U.S. Pat. No. 4,021,423, U.S. Pat. No. 4,026,882, U.S. Pat. No. 4,028,349, U.S. Pat. No. 4,225,525, U.S. Pat. No. 4,613,594, U.S. Pat. No. 4,804,502, U.S. Pat. No. 4,898,855, U.S. Pat. No. 5,039,671, U.S. Pat. No. 5,087,619, U.S. Pat. No. 5,145,846, U.S. Pat. No. 5,247,123, U.S. Pat. No. 5,342,833, U.S. Pat. No. 5,428,029, U.S. Pat. No. 5,451,574, U.S. Pat. No. 5,612,328, U.S. Pat. No. 5,747,479, U.S. Pat. No. 5,804,574, U.S. Pat. No. 5,811,414, U.S. Pat. No. 5,856,317, U.S. Pat. No. 5,872,113, U.S. Pat. No. 5,888,994, U.S. Pat. No. 5,939,408, U.S. Pat. No. 5,962,707, U.S. Pat. No. 5,981,780, U.S. Pat. No. 6,017,908, U.S. Pat. No. 6,030,962, U.S. Pat. No. 6,040,461, U.S. Pat. No. 6,100,294, U.S. Pat. No. 6,121,312, U.S. Pat. No. 6,329,538, U.S. Pat. No. 6,331,642, U.S. Pat. No. 6,392,071, U.S. Pat. No. 6,452,028, U.S. Pat. No. 6,479,538, U.S. Pat. No. 6,492,353, U.S. Pat. No. 6,537,981, U.S. Pat. No. 6,544,969, U.S. Pat. No. 6,559,138, U.S. Pat. No. 6,667,298, U.S. Pat. No. 6,683,219, U.S. Pat. No. 6,696,431, U.S. Pat. No. 6,774,251, and those described in published US patent applications: US2001007907, US2003083319, US2003125309, US2003130241, US2003171605, US2004167105.

It will be noted that the structures of some of the compounds of the invention include asymmetric carbon atoms. Accordingly, it is to be understood that the isomers arising from such asymmetry (e.g., all enantiomers and diastereomers) are included within the scope of this invention, unless indicated otherwise. Such isomers can be obtained in substantially pure form by classical separation techniques and/or by stereochemically controlled synthesis.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

The invention also provides a pharmaceutical composition, comprising an effective amount of a vitamin D compound as described herein and a pharmaceutically acceptable carrier. In a further embodiment, the effective amount is effective to treat interstitial cystitis, as described previously.

In an embodiment, the vitamin D compound is administered to the subject using a pharmaceutically-acceptable formulation, e.g., a pharmaceutically-acceptable formulation that provides sustained delivery of the vitamin D compound to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension, (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those vitamin D compounds of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a vitamin D compound(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these compositions include the step of bringing into association a vitamin D compound(s) with the carrier and, optionally, one or more accessory ingredients. In general, the formulations are prepared by uniformly and intimately bringing into association a vitamin D compound with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a vitamin D compound(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the vitamin D compound(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active vitamin D compound(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more vitamin D compound(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a vitamin D compound(s) include powders, sprays, ointments, pastes, creams, lotions, gels, solutions, patches and inhalants. The active vitamin D compound(s) may be mixed under sterile conditions with a pharmaceutically-acceptable carrier, and with any preservatives, buffers, or propellants which may be required.

The ointments, pastes, creams and gels may contain, in addition to vitamin D compound(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a vitamin D compound(s), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The vitamin D compound(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a vitamin D compound(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Pharmaceutical compositions of the invention suitable for parenteral administration comprise one or more vitamin D compound(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of vitamin D compound(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the vitamin D compound(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the vitamin D compound(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of the invention may be varied so as to obtain an amount of the active ingredient which is effective to achieve the desired therapeutic response for a particular patient, composition, and mode of administration, without being toxic to the patient. An exemplary dose range is from 0.1 to 300 μg per day

A preferred dose of the vitamin D compound for the present invention is the maximum that a patient can tolerate and not develop hypercalcemia. Preferably, the vitamin D compound of the present invention is administered at a concentration of about 0.001 ug to about 100 ug per kilogram of body weight, about 0.001-about 10 ug/kg or about 0.001 ug-about 100 ug/kg of body weight. Ranges intermediate to the above-recited values are also intended to be part of the invention.

The vitamin D compound may be administered separately, sequentially or simultaneously in separate or combined pharmaceutical formulations with a second medicament for the treatment of interstitial cystitis.

Synthesis of Compounds of the Invention

A number of the compounds of the present invention can be prepared by incubation of vitamin D₃ analogues in cells, for example, incubation of vitamin D₃ analogues in either UMR 106 cells or Ros 17/2.8 cells results in production of vitamin D₃ compounds of the invention. For example, Incubation of 1,25-dihydroxy-16-ene-5,6-trans-calcitriol in UMR 106 cells results in production of the 1,25-dihydroxy-16-ene-24-oxo-5,6-trans-calcitriol.

In addition to the methods described herein, compounds of the present invention can be prepared using a variety of synthetic methods. For example, one skilled in the art would be able to use methods for synthesizing existing vitamin D₃ compounds to prepare compounds of the invention (see e.g., Bouillon, R. et al., (1995) Endocrine Reviews 16(2):201-204; Ikekawa N. (1987) Med. Res. Rev. 7:333-366; DeLuca H. F. and Ostrem V. K. (1988) Prog. Clin. Biol. Res. 259:41-55; Ikekawa N. and Ishizuka S. (1992) CRC Press 8:293-316; Calverley M. J. and Jones G. (1992) Academic Press 193-270; Pardo R. and Santelli M. (1985) Bull. Soc. Chim. Fr:98-114; Bythgoe B. (1980) Chem. Soc. Rev. 449-475; Quinkert G. (1985) Synform 3:41-122; Quinkert G. (1986) Synform 4:131-256; Quinkert G. (1987) Synform 5:1-85; Mathieu C. et al. (1994) Diabetologia 37:552-558; Dai H. and Posner G. H. (1994) Synthesis 1383-1398); DeLuca et al., WO 97/11053.

Exemplary methods of synthesis include the photochemical ring opening of a 1-hydroxylated side chain-modified derivative of 7-dehydrocholesterol which initially produces a previtamin that is easily thermolyzed to vitamin D₃ in a well known fashion (Barton D. H. R. et al. (1973) J. Am. Chem. Soc. 95:2748-2749; Barton D. H. R. (1974) JCS Chem. Comm. 203-204); phosphine oxide coupling method developed by (Lythgoe et al (1978) JCS Perkin Trans. 1:590-595) which comprises coupling a phosphine oxide to a Grundmann's ketone derivative to directly produce a 1-alpha,25(OH)₂D₃ skeleton as described in Baggiolini E. G., et al. (1986) J. Org. Chem. 51:3098-3108; DeSchrijver J. and DeClercq P. J. (1993) Tetrahed Lett 34:4369-4372; Posner G. H and Kinter C. M. (1990) J. Org. Chem. 55:3967-3969; semihydrogenation of dienynes to a previtamin structure that undergoes rearrangement to the corresponding vitamin D₃ analogue as described by Harrison R. G. et al. (1974) JCS Perkin Trans. 1:2654-2657; Castedo L. et al. (1988) Tetrahed Lett 29:1203-1206; Mascarenas J. S. (1991) Tetrahedron 47:3485-3498; Barrack S. A. et al. (1988) J. Org. Chem. 53:1790-1796) and Okamura W. H. et al. (1989) J. Org. Chem. 54:4072-4083; the vinylallene approach involving intermediates that are subsequently arranged using heat or a combination of metal catalyzed isomerization followed by sensitized photoisomerization (Okamura W. H. et al. (1989) J. Org. Chem. 54:4072-4083; Van Alstyne E. M. et al. (1994) J. Am. Chem. Soc. 116:6207-6210); the method described by Trost et al. B. M. et al. J. Am. Chem. Soc. 114:9836-9845; Nagasawa K. et al. (1991) Tetrahed Lett 32:4937-4940 involves an acyclic A-ring precursor which is intramolecular cross-coupled to the bromoenyne leading directly to the formation of 1,25(OH)₂D₃ skeleton; a tosylated derivative which is isomerized to the i-steroid that can be modified at carbon-1 and then subsequently back-isomerized under sovolytic conditions to form 1-alpha,25(OH)₂D₂ or analogues thereof (Sheves M. and Mazur Y. (1974) J. Am. Chem. Soc. 97:6249-6250; Paaren H. E. et al. (1980) J. Org. Chem. 45:3253-3258; Kabat M. et al. (1991) Tetrahed Lett 32:2343-2346; Wilson S. R. et al. (1991) Tetrahed Lett 32:2339-2342); the direct modification of vitamin D derivatives to 1-oxygenated 5,6-trans vitamin D as described in (Andrews D. R. et al. (1986) J. Org. Chem. 51:1635-1637); the Diels-Alders cycloadduct method of previtamin D₃ can be used to cyclorevert to 1-alpha,25(OH)₂D₂ through the intermediary of a previtamin form via thermal isomerization (Vanmaele L. et al. (1985) Tetrahedron 41:141-144); and, a final method entails the direct modification of 1-alpha,25(OH)₂D₂ or an analogue through use of suitable protecting groups such as transition metal derivatives or by other chemical transformations (Okarmura W. H. et al. (1992) J. Cell Biochem. 49:10-18). Additional methods for synthesizing vitamins D2 compounds are described in, for example, Japanese Patent Disclosures Nos. 62750/73, 26858/76, 26859/76, and 71456/77; U.S. Pat. Nos. 3,639,596; 3,715,374; 3,847,955 and 3,739,001.

Examples of the compounds of this invention having a saturated side chain can be prepared according to the general process illustrated and described in U.S. Pat. No. 4,927,815. Examples of compounds of the invention having an unsaturated side chain can be prepared according to the general process illustrated and described in U.S. Pat. No. 4,847,012. Examples of compounds of the invention wherein R groups together represent a cycloalkyl group can be prepared according to the general process illustrated and described in U.S. Pat. No. 4,851,401.

Another synthetic strategy for the preparation of side-chain-modified analogues of 1-alpha,25-dihydroxyergocalciferol is disclosed in Kutner et al., The Journal of Organic Chemistry, 1988, 53:3450-3457. In addition, the preparation of 24-homo and 26-homo vitamin D analogues are disclosed in U.S. Pat. No. 4,717,721.

The enantioselective synthesis of chiral molecules is now state of the art. Through combinations of enantioselective synthesis and purification techniques, many chiral molecules can be synthesized as an enantiomerically enriched preparation. For example, methods have been reported for the enantioselective synthesis of A-ring diastereomers of 1-alpha,25(OH)₂D₃ as described in Muralidharan et al (1993) J. Organic Chem. 58(7): 1895-1899 and Norman et al. (1993) J. Biol. Chem. 268(27): 20022-30. Other methods for the enantiomeric synthesis of various compounds known in the art include, inter alia, epoxides (see, e.g., Johnson, R. A.; Sharpless, K. B. In Catalytic Asymmetric Synthesis; Ojima, I., Ed.: VCH: New York, 1993; Chapter 4.1. Jacobsen, E. N. Ibid. Chapter 4.2), diols (e.g., by the method of Sharpless, J. Org. Chem. (1992) 57:2768), and alcohols (e.g., by reduction of ketones, E. J. Corey et al., J. Am. Chem. Soc. (1987) 109:5551). Other reactions useful for generating optically enriched products include hydrogenation of olefins (e.g., M. Kitamura et al., J. Org. Chem. (1988) 53:708); Diels-Alder reactions (e.g., K. Narasaka et al., J. Am. Chem. Soc. (1989) 111:5340); aldol reactions and alkylation of enolates (see, e.g., D. A. Evans et al., J. Am. Chem. Soc. (1981) 103:2127; D. A. Evans et al., J. Am. Chem. Soc. (1982) 104:1737); carbonyl additions (e.g., R. Noyori, Angew. Chem. Int. Ed. Eng. (1991) 30:49); and ring-opening of meso-epoxides (e.g., Martinez, L. E.; Leighton J. L., Carsten, D. H.; Jacobsen, E. N. J. Am. Chem. Soc. (1995) 117:5897-5898). The use of enzymes to produce optically enriched products is also well known in the art (e.g., M. P. Scheider, ed. “Enzymes as Catalysts in Organic Synthesis”, D. Reidel, Dordrecht (1986).

Chiral synthesis can result in products of high stereoisomer purity. However, in some cases, the stereoisomer purity of the product is not sufficiently high. The skilled artisan will appreciate that the separation methods described herein can be used to further enhance the stereoisomer purity of the vitamin D₃-epimer obtained by chiral synthesis.

Compounds of formula (XVIII): 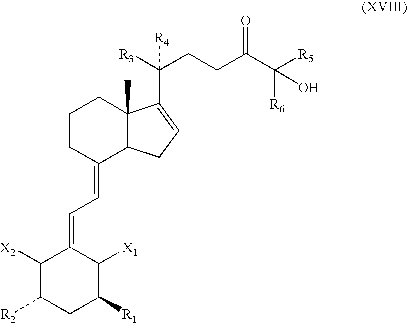
wherein:
X₁ and X₁ are each independently H₂ or ═CH₂, provided X₁ and X₁ are not both ═CH₂;
R₁ and R₂ are each independently, hydroxyl, OC(O)C₁-C₄ alkyl, OC(O)hydroxyalkyl, OC(O)fluororalkyl, provided that R₁ and R₂ are not both hydroxyl;
R₃ and R₄ are each independently hydrogen, C₁-C₄ alkyl, or R₃ and R₄ taken together with C₂₀ form C₃-C₆ cycloalkyl; and
R₅ and R₆ are each independently C₁-C₄ alkyl, hydroxyalkyl, or haloalkyl, e.g., fluoroalkyl, e.g., fluoromethyl and trifluoromethyl;

and pharmaceutically acceptable esters, salts, and prodrugs thereof, can be synthesized by methods described in this section, and the chemical literature. In particular, compounds of formula (XVIII) of the invention are prepared as shown in Scheme 1 below. Accordingly, compounds of formula (XVIII) are prepared by coupling compounds of formula (XIX) with compounds of formula (XX) in tetrahydrofuran with n-butyllithium as a base to give compounds of formula (XXI). Subsequent removal of the protecting silyl groups (R₁═OSi(CH₃)₂t.Bu) affords the 1,3 dihydroxy vitamin D₃ compound of formula (XVIII) (R₁═OH, R₂═OH). Acylation at the 1 and/or 3 positions is achieved using methods well-known in the art. For example, preparation of the 1,3 diacetoxy compounds of formula IV (R₁═R₂═OAc) requires additional acetylation with acetic anhydride and pyridine, as shown in Scheme 2 and described below.

Referring to Scheme 1, compounds of formula (XX) are known compounds, and are prepared starting from the known epoxy-ketone of formula (XXII). The compound of formula (XXII) is converted to the epoxy-olefin of formula (XXIII) by a Wittig reaction. Reduction with LiAlH₄ to the compound (XXIV) and protection of the hydroxy group resulted in compound (XXV). Then, the ene reaction of formula (XXV) with the known hydroxy-conjugated ketone (XXVI) (R₅═R₆═CH₃) in tetrahydrofuran, in the presence of Lewis acid (CH₃)₂ Al Cl, provides the compound (XXVII) featuring the C,D-rings and full side chain of the target vitamin D analogs. Finally, removal of the silyl group and oxidation provides the key intermediate, Ketone of formula (XX).

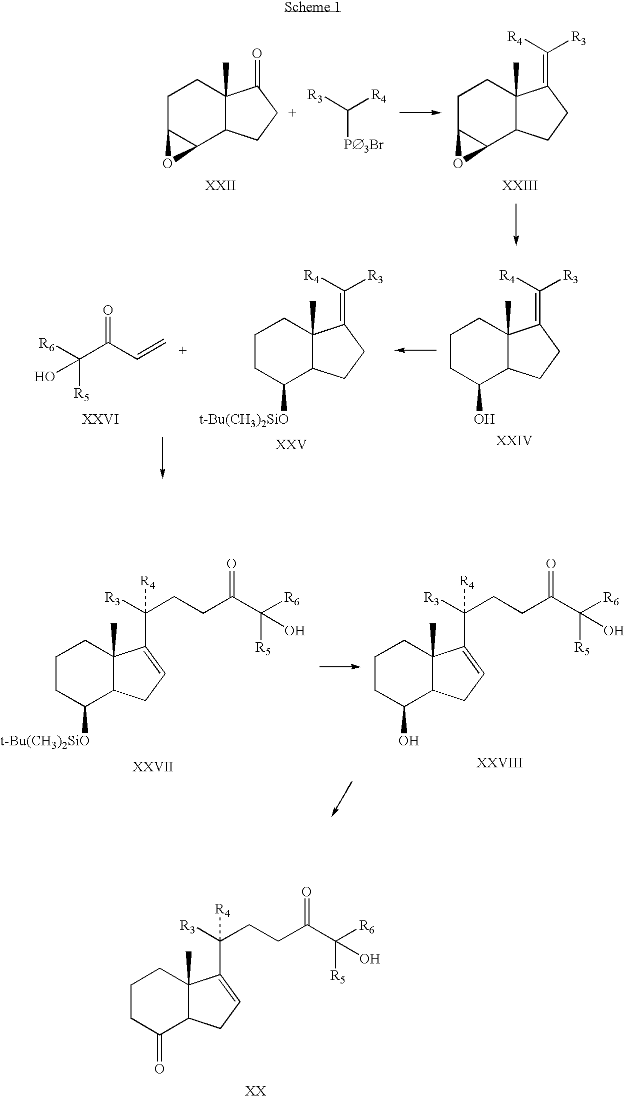

Scheme 2 shows the coupling of compound (XX) with a silylated phosphine oxide under Witting coupling conditions. Removal of the silyl protecting group provides diols of formula (XVIII), where R₁ and R² are both hydroxyl. 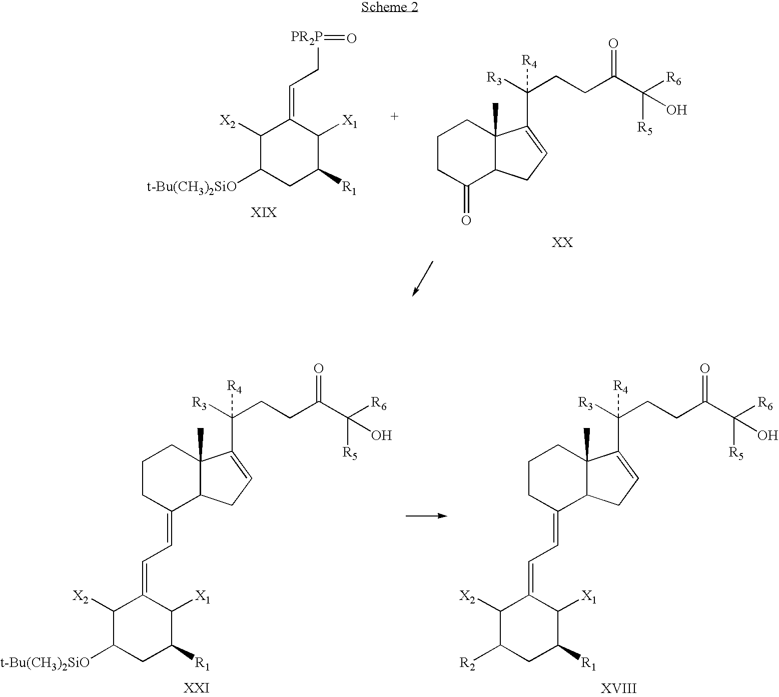
wherein X₁, X₂, R₃, R₄, R₅ and R₆ are as defined above.

Scheme 3 demonstrates the acetylation of the vitamin D₃ derivatives of formula (P) to the acetates of formula (Q).

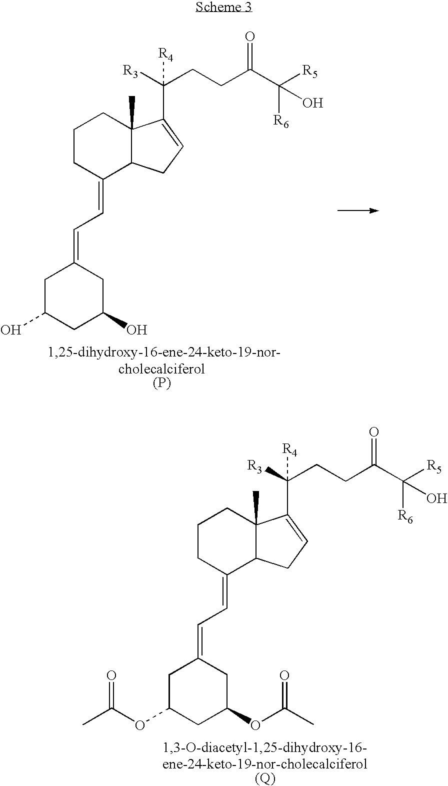

Vitamin D₃ compounds of the formula: 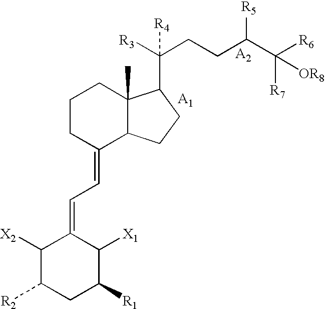
wherein:
A₁ is single or double bond;
A₂ is a single, double or triple bond;
X₁ and X₂ are each independently H or ═CH₂,
R₁ and R₂ are each independently OC(O)C1-C4 alkyl, OC(O)hydroxyalkyl, or OROC(O)haloalkyl;
R₃, R₄ and R₅ are each independently hydrogen, C1-C4 alkyl, hydroxyalkyl, or haloalkyl, or R₃ and R₄ taken together with C20 form C3-C6 cycloalkyl;
R₆ and R₇ are each independently haloalkyl; and

R₈ is H or OC(O)CL-C4 alkyl, OC(O)hydroxyalkyl, or OROC(O)haloalkyl; and

pharmaceutically acceptable esters, salts, and prodrugs thereof.
may be prepared analogously to the synthesis of 1,3-Di-O-acetyl-1,25-dihydroxy-16,23Z-diene-26,27-hexafluoro-19-nor-cholecalciferol (1) (“Compound A” in the following examples), which is carried out under standard acetylation conditions of the diol to the corresponding diacetate:

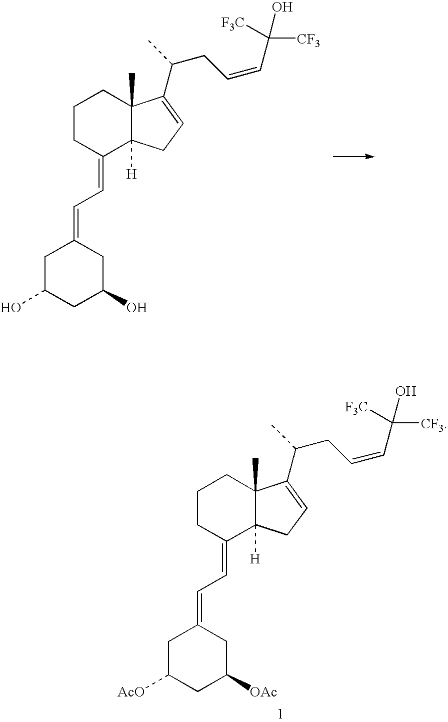

The present invention will now be described with reference to the following non-limiting examples, with reference to the figures, in which:

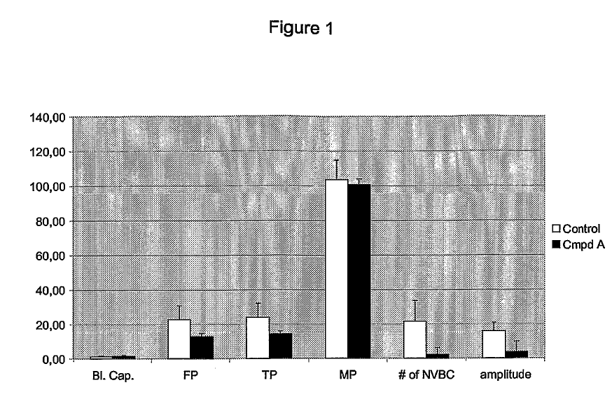

FIG. 1 shows a comparison between cystometric parameters recorded in rats treated with a vitamin D3 analogue “Compound A” and control (vehicle treated) rats.

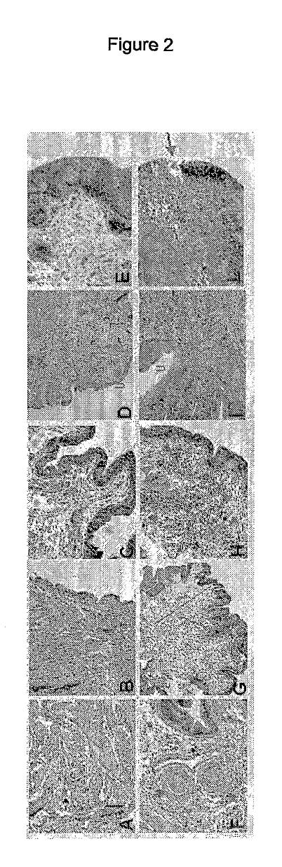

FIG. 2 shows the effect of a vitamin D3 analogue Compound A (A-E) versus vehicle (miglyol) (F-L) on the histological signs of inflammation in rat bladders. Five different parameters were considered: hemostasis (A,F), edema (B,G), infiltration (C,H), fibrosis (D,I), urothelial damage (E,L). Arrows and bars indicate the signs of inflammation present in the vehicle treated animal versus Compound A treated rats. U=urothelium.

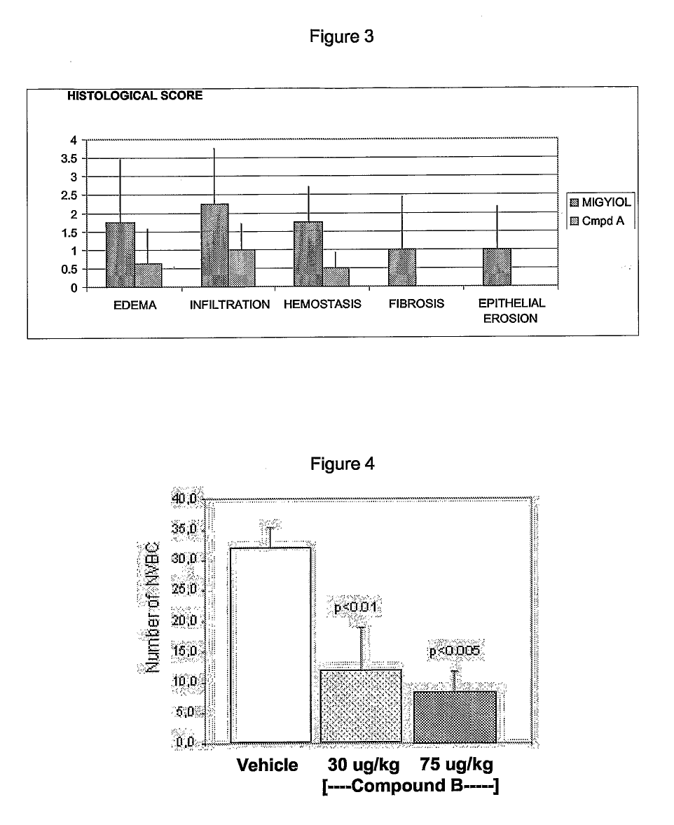

FIG. 3 shows a histogram summarizing the histological score of 4 rats per group for each sign of inflammation. Different inflammatory parameters were considered: hemostasis, edema, infiltration of inflammatory cells (mostly lymphocyte and monocyte), epithelial erosion, fibrosis and scored as described in Example 2. The mean of histological scores ±standard deviation was plotted.

FIG. 4 shows the number of non-voiding bladder contractions experienced by subjects from Example 5. The average number of contractions are shown for each treatment group (vehicle control, 30 ug/kg Compound B and 75 ug/kg Compound B) with error bars indicating the standard deviation.

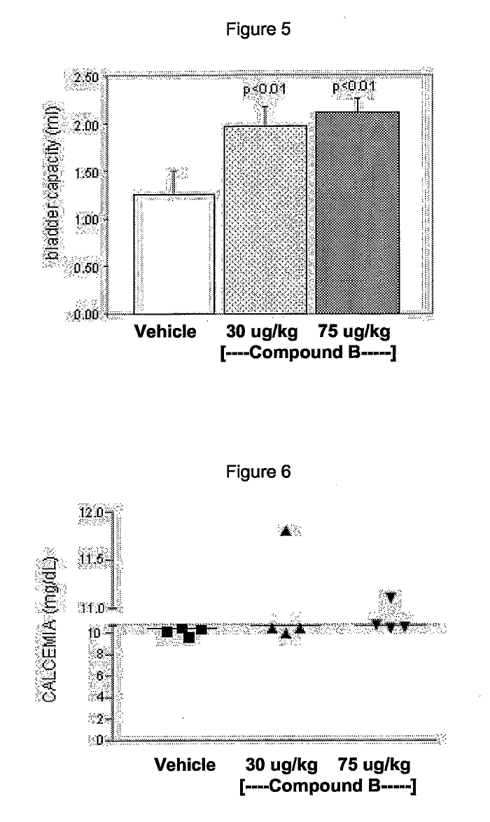

FIG. 5 shows the bladder capacity of subjects from Example 5. The average bladder capacity (ml) is shown for each treatment group (vehicle control, 30 ug/kg Compound B and 75 ug/kg Compound B) with error bars indicating the standard deviation.

FIG. 6 shows the serum calcium levels of subjects from Example 5. The calcium levels in serum are shown (mg/dL) for each subject in the treatment groups (vehicle control, 30 ug/kg Compound B and 75 ug/kg Compound B) with the average level in each group indicated by a horizontal line.

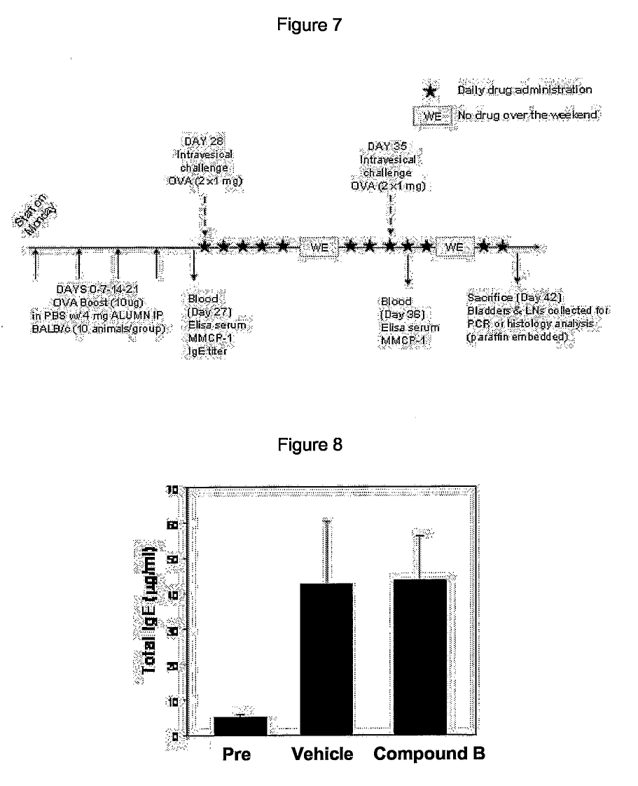

FIG. 7 illustrates the experimental timeline for Example 6.

FIG. 8 shows the total amount of IgE protein detected in serum of subjects from Example 6. The average total amount of IgE (ug/ml) is shown for each group (pre challenge, vehicle treated control, 75 ug/kg Compound B treated) with error bars indicating the standard deviation.

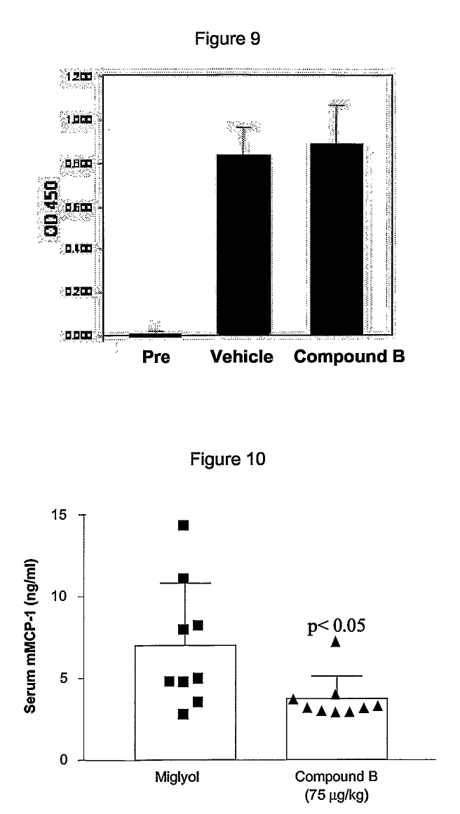

FIG. 9 shows the amount of ovalbumin specific IgE protein detected in serum of subjects from Example 6. The average amount of specific IgE protein in serum (OD450) is shown for each group (pre challenge, vehicle treated control, 75 ug/kg Compound B treated) with error bars indicating the standard deviation.

FIG. 10 shows the serum MMCP1 protein levels in subjects from Example 6. The average level of serum MMCP1 protein (ug/ml) is shown for each group (pre challenge, vehicle treated control, 75 ug/kg Compound B treated) with error bars indicating the standard deviation. This data is overlaid with the individual values derived from each subject.

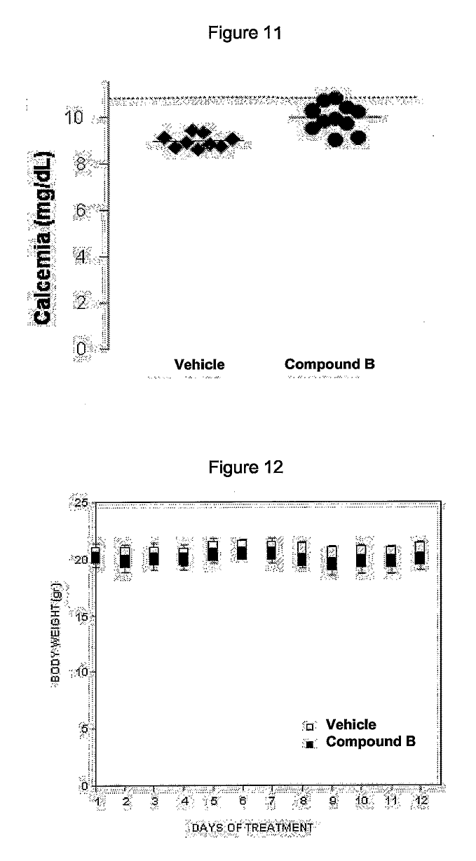

FIG. 11 shows the serum calcium levels of subjects from Example 6. The calcium levels in serum are shown (mg/dL) for each subject in the treatment groups (vehicle control and 75 ug/kg Compound B) with the average level in each group indicated by a horizontal line. An extended dashed horizontal line indicates the level at which calcium toxicity begins to arise.

FIG. 12 shows the variation in body weight of subjects from Example 6. The average body weight is shown (g) at daily timepoints for both treatment groups (vehicle control and 75 ug/kg Compound B), error bars indicate the standard deviation.

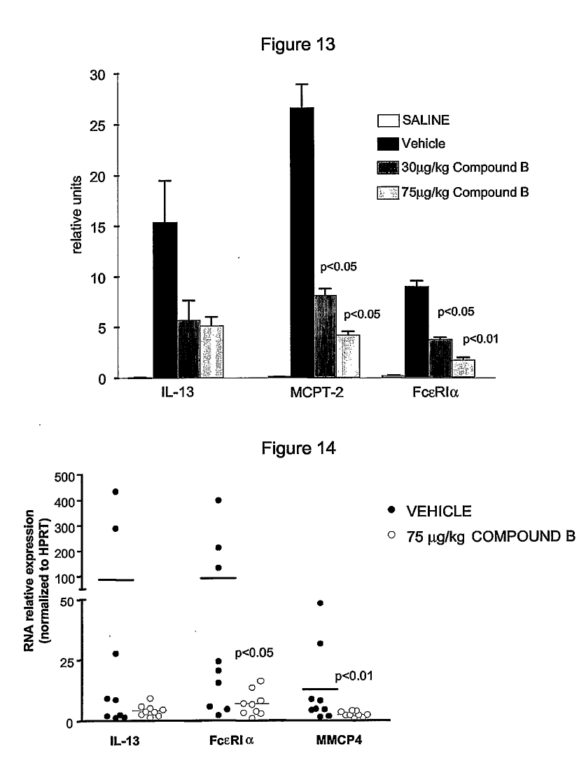

FIG. 13 shows the mRNA expression levels of the inflammatory marker genes IL-13, MCPT2 and FcεR1α in subjects from Example 6. Data shows the level of each marker relative to the housekeeping gene for saline challenged (vehicle treated) and ovalbumin challenged (vehicle, 30 ug/kg Compound B and 75 ug/kg Compound B treated).

FIG. 14 shows the mRNA expression levels of the inflammatory marker genes IL-13, MMCP4 and FcεR1α in subjects from Example 6. The plot shows the level of each marker relative to the housekeeping gene for ovalbumin challenged treatment groups (vehicle or 75 ug/kg Compound B). The individual data points from subjects are plotted, with a horizontal line indicating the average level.

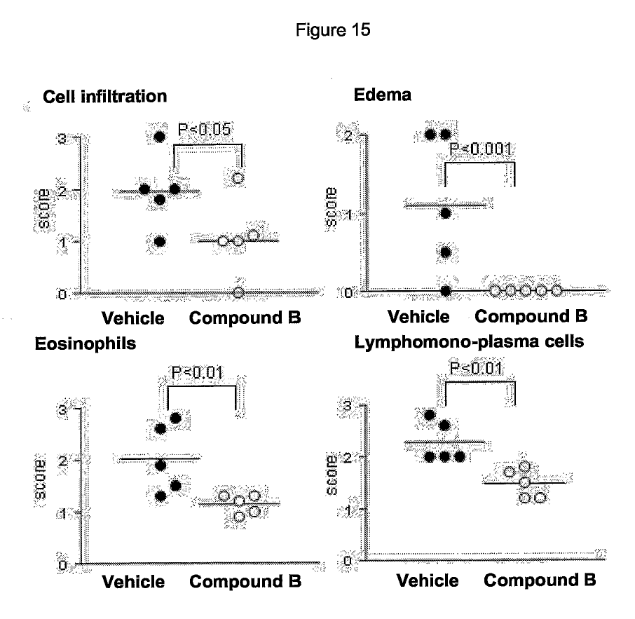

FIG. 15 shows the results of histological analysis (Mast cell infiltration, Edema, Eosinophils and Lymphomono-plasma cells) performed on subjects from Example 6. The plots show the score allocated to each subject, with the average level for each treatment group (vehicle or 75 ug/kg Compound B) indicated by a horizontal line.

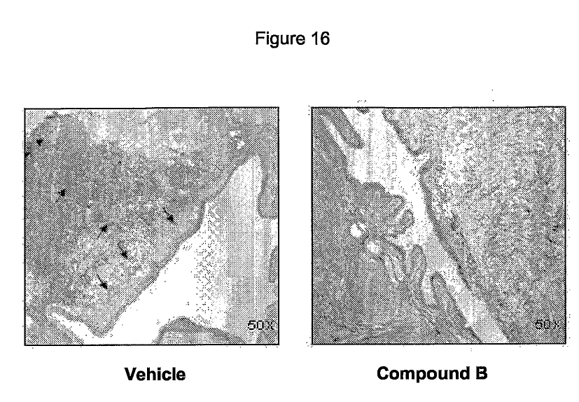

FIG. 16 shows representative sections of bladder (×50 magnification) from vehicle and 75 ug/kg Compound B treated animals. Histological lesions are indicated with arrows.

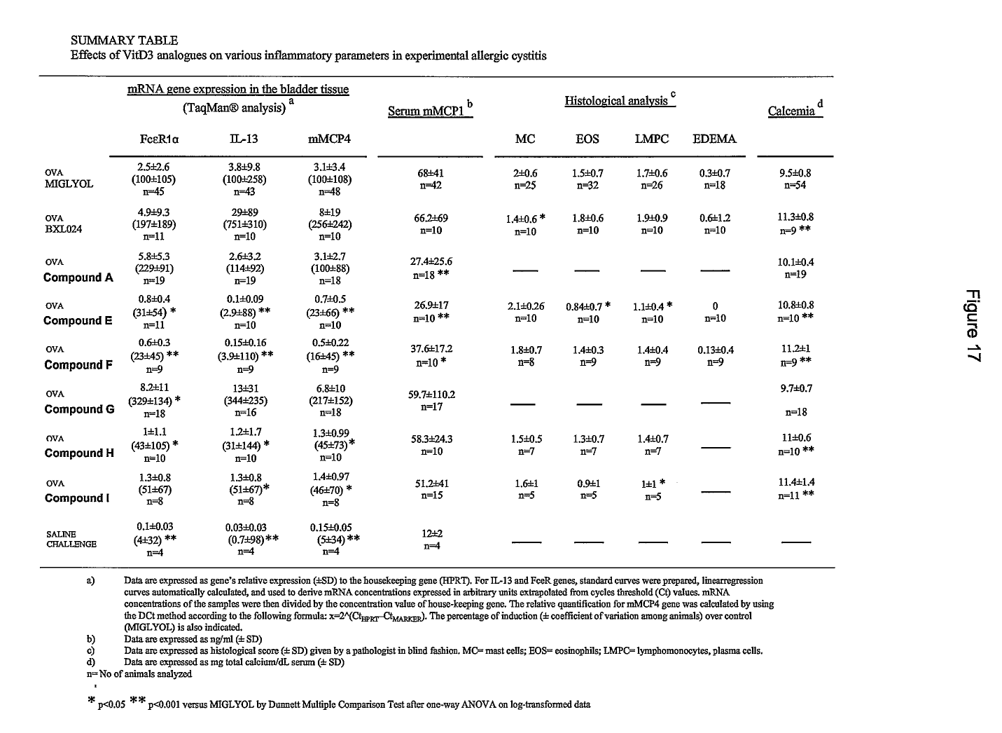

FIG. 17 shows a summary of the experimental results from Example 7.

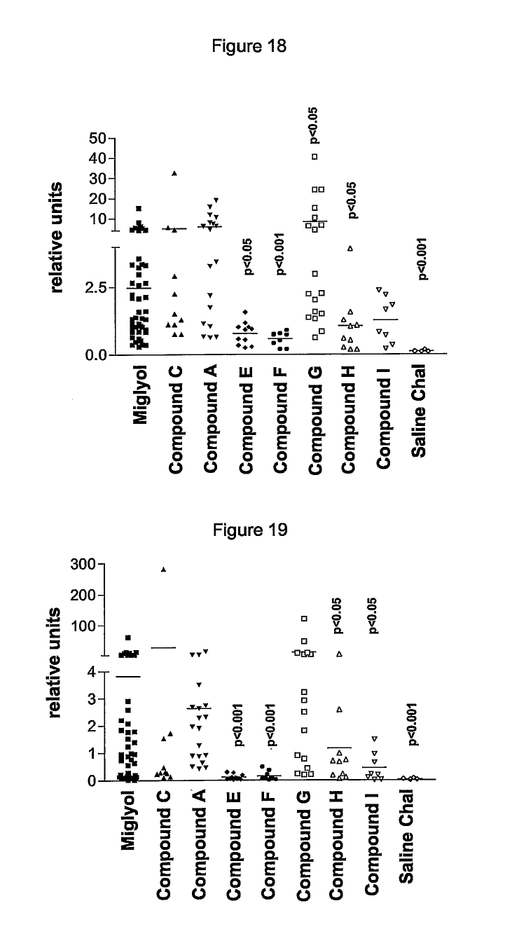

FIG. 18 shows the mRNA expression level of the inflammatory marker gene FcεR1α in subjects from Example 7. The plot shows the level of FcεR1α relative to the housekeeping gene for saline challenged, untreated and ovalbumin challenged treatment groups (vehicle, Compound C to Compound I treated). The individual data points from subjects are plotted, with a horizontal line indicating the average level.

FIG. 19 shows the mRNA expression level of the inflammatory marker gene IL-13 in subjects from Example 7. The plot shows the level of IL-13 relative to the housekeeping gene for saline challenged, untreated and ovalbumin challenged treatment groups (vehicle, Compound C to Compound I treated). The individual data points from subjects are plotted, with a horizontal line indicating the average level.

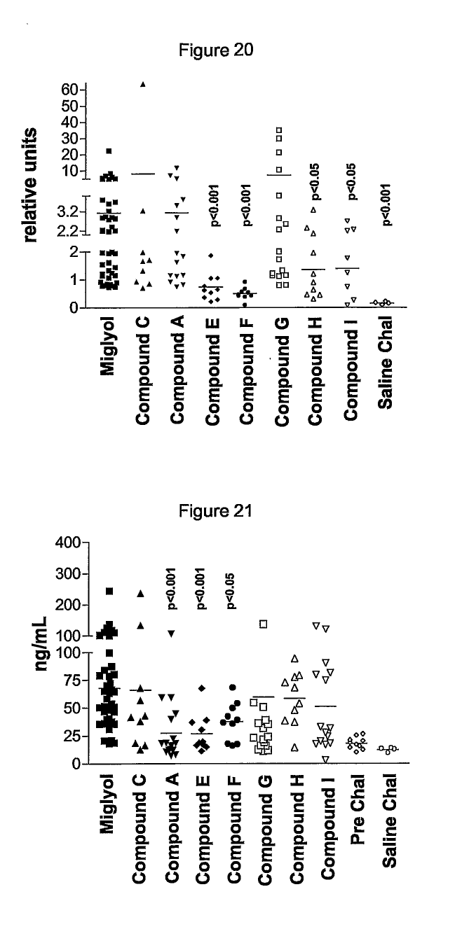

FIG. 20 shows the mRNA expression level of the inflammatory marker gene MMCP4 in subjects from Example 7. The plot shows the level of MMPC4 relative to the housekeeping gene for saline challenged, untreated and ovalbumin challenged treatment groups (vehicle, Compound C to Compound I treated). The individual data points from subjects are plotted, with a horizontal line indicating the average level.

FIG. 21 shows the serum MMCP1 protein levels in subjects from Example 7. The plot shows the level of MMCP1 protein (ng/ml) in serum for pre-challenge, saline challenged, untreated and ovalbumin challenged treatment groups (vehicle, Compound C to Compound I treated). The individual data points from subjects are plotted, with a horizontal line indicating the average level

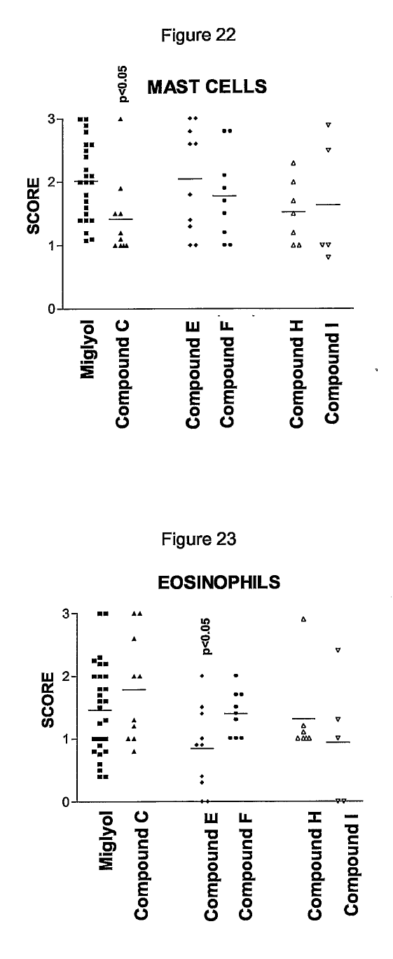

FIG. 22 shows the results of histological analysis of mast cell infiltration performed on subjects from Example 7. The plot shows the score allocated to each subject, with the average level for each treatment group (vehicle, Compound C, Compound E, Compound F, Compound H or Compound I) indicated by a horizontal line.

FIG. 23 shows the results of histological analysis of eosinophils performed on subjects from Example 7. The plot shows the score allocated to each subject, with the average level for each treatment group (vehicle, Compound C, Compound E, Compound F, Compound H or Compound I) indicated by a horizontal line.

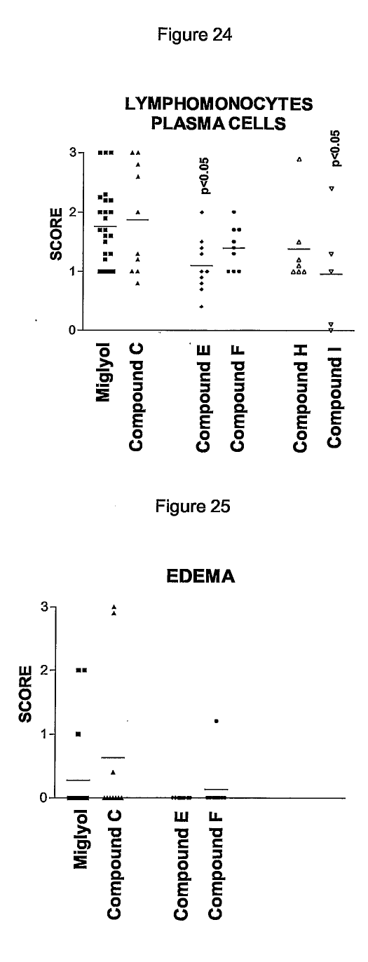

FIG. 24 shows the results of histological analysis of LMPC performed on subjects from Example 7. The plot shows the score allocated to each subject, with the average level for each treatment group (vehicle, Compound C, Compound E, Compound F, Compound H or Compound I) indicated by a horizontal line.

FIG. 25 shows the results of histological analysis of edema performed on subjects from Example 7. The plot shows the score allocated to each subject, with the average level for each treatment group (vehicle, Compound C, Compound E or Compound F) indicated by a horizontal line.

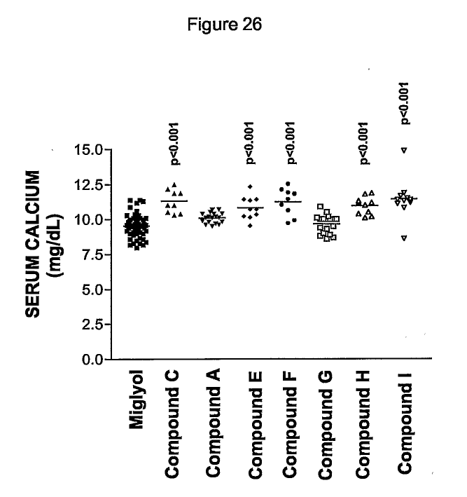

FIG. 26 shows the calcium concentration in serum of subjects from Example 7. The serum calcium concentrations are shown (mg/dL) for each subject in the treatment groups (vehicle control, Compounds C to I) with the average level in each group indicated by a horizontal line.

### SYNTHETIC EXAMPLES

All operations involving vitamin D₃ analogs were conducted in amber-colored glassware in a nitrogen atmosphere. Tetrahydrofuran was distilled from sodium-benzophenone ketyl just prior to its use and solutions of solutes were dried with sodium sulfate. Melting points were determined on a Thomas-Hoover capillary apparatus and are uncorrected. Optical rotations were measured at 25° C. ¹H NMR spectra were recorded at 400 MHz in CDCl₃ unless indicated otherwise. TLC was carried out on silica gel plates (Merck PF-254) with visualization under short-wavelength UV light or by spraying the plates with 10% phosphomolybdic acid in methanol followed by heating. Flash chromatography was carried out on 40-65 μm mesh silica gel. Preparative HPLC was performed on a 5×50 cm column and 15-30 μm mesh silica gel at a flow rate of 100 ml/min.

### Synthetic Example 1

### Synthesis of 1,3-Di-O-acetyl-1,25-dihydroxy-16,23Z-diene-26,27-hexafluoro-19-nor-cholecalciferol (1) (Compound A)

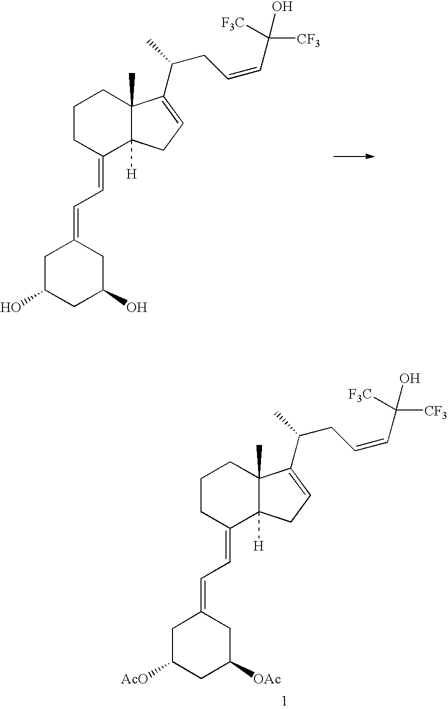

The starting material 1,25-dihydroxy-16,23Z-diene-26,27-hexafluoro-19-nor-cholecalciferol can be prepared as described in U.S. Pat. No. 5,428,029 to Doran et al. 3 mg of 1,25-dihydroxy-16,23Z-diene-26,27-hexafluoro-19-nor-cholecalciferol was dissolved in 0.8 ml of pyridine, cooled to ice-bath temperature and 0.2 ml of acetic anhydride was added and maintained at that temperature for 16 h. Then the reaction mixture was diluted with 1 ml of water, stirred for 10 min in the ice bath and distributed between 5 ml of water and 20 ml of ethyl acetate. The organic layer was washed with 3×5 ml of water, once with 5 ml of saturated sodium hydrogen carbonate, once with 3 ml of brine then dried (sodium sulfate) and evaporated. The oily residue was taken up in 1:6 ethyl acetate-hexane and flash-chromatographed using a stepwise gradient of 1:6, 1:4 and 1:2 ethyl acetate-hexane. The column chromatography was monitored by TLC (1:4 ethyl acetate-hexane, spot visualization with phosphomolybdic acid spray), the appropriate fractions were pooled, evaporated, the residue taken up in methyl formate, filtered, then evaporated again to give 23.8 mg of the title compound (1) as a colorless syrup; 400 MHz ¹H NMR δ 0.66 (3H, s), 0.90 (1H, m), 1.06 (3H, d, J=7.2 Hz), 1.51 (1H, m), 1.72-1.82 (3H, m), 1.9-2.1 (3H, m), 1.99 (3H, s) 2.04 (3H, s), 2.2-2.3 (3 m), 2.44-2.64 (6H, m), 2.78 (1H, m), 3.01 (1H, s), 5.10 (2H, m). 5.38 (1H, m), 5.43 (1H, d, J=12 Hz), 5.85 (1H, d, J=11.5 Hz), 5.97 (1H, dt, J=12 and 7.3 Hz), 6.25 (1H, d, J=11.5 Hz).

### Synthetic Example 2

### Synthesis of 1,3-Di-O-acetyl-1,25-Dihydroxy-16-ene-23-yne-26,27-hexafluoro-19-nor-cholecalciferol (2) and 1,3,25-Tri-O-acetyl-1,25-Dihydroxy-16-ene-23-yne-26,27-hexafluoro-19-nor-cholecalciferol (3)

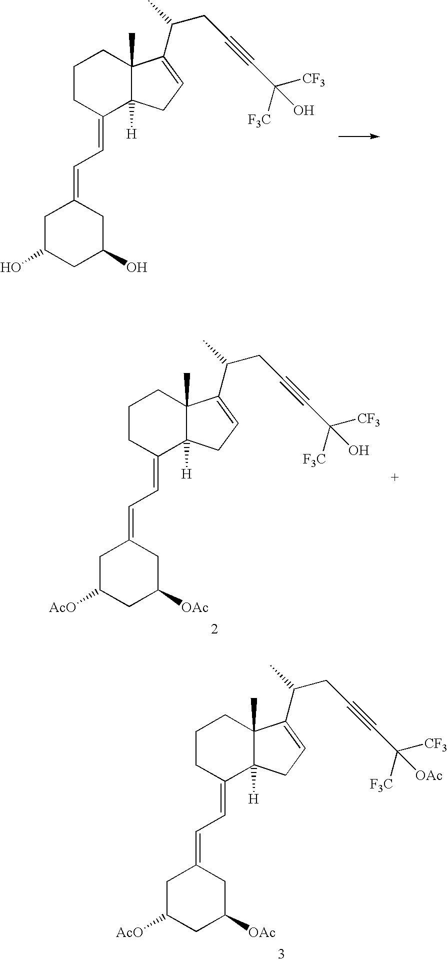

The starting material 1,25-dihydroxy-16-ene-23-yne-26,27-hexafluoro-19-nor-cholecalciferol can be prepared as described in U.S. Pat. Nos. 5,451,574 and 5,612,328 to Baggiolini et al. 314 mg (0.619 mmole) of 1,25-dihydroxy-16-ene-23-yne-26,27-hexafluoro-19-nor-cholecalciferol was dissolved in 1.5 ml of pyridine, cooled to ice-bath temperature, and 0.4 ml of acetic anhydride was added. The reaction mixture was kept at room temperature for 7 hours and then for 23 hours in a refrigerator. It was then diluted with 10 ml water and extracted with 30 ml of ethyl acetate. The organic extract was washed with water and brine, dried over sodium sulfate and evaporated. The residue was FLASH chromatographed on a 10×140 mm column with 1:6 and 1:4 ethyl acetate-hexane as the mobile phase to give 126 mg of 1,3-Di-O-acetyl-1,25-Dihydroxy-16-ene-23-yne-26,27-hexafluoro-19-nor-cholecalciferol (2), and 248 mg of 1,3,25-Tri-O-acetyl-1,25-Dihydroxy-16-ene-23-yne-26,27-hexafluoro-19-nor-cholecalciferol (3).

### Synthetic Example 3

### Synthesis of 1,3-Di-O-acetyl-1,25-dihydroxy-16-ene-23-yne-cholecalciferol (4)

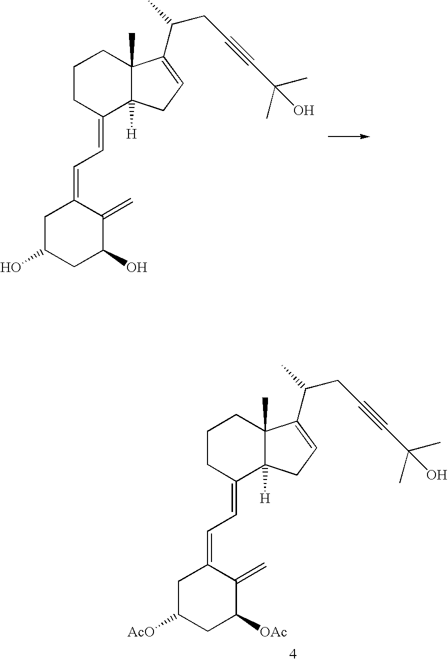

A 10-mL round-bottom flask was charged with 40 mg of 1,25-dihydroxy-16-ene-23-yne-cholecalciferol. This material was dissolved in 1 mL of pyridine. This solution was cooled in an ice bath then 0.3 mL of acetic anhydride was added. The solution was stirred for 30 min, then refrigerated overnight, diluted with water and transferred to a separatory funnel with the aid of 10 mL of water and 40 mL of ethyl acetate. The organic layer was washed with 4×20 mL of water, 10 mL of brine passed through a plug of sodium sulfate and evaporated. The light brown, oily residue was taken up in 1:9 ethyl acetate-hexane then flash chromatographed on a 10×130 mm column using 1:9 ethyl acetate-hexane as mobile phase for fractions 1-5, 1:6 for fractions 6-13 and 1:4 ethyl acetate-hexane for fractions 14-20 (18 mL fractions). Fractions 14-19 contained the main band with Rf0.15 (TLC 1:4). Those fractions were pooled and evaporated to a colorless oil, 0.044 g. The material was taken up in methyl formate, filtered and evaporated to give a colorless, sticky foam, 0.0414 g of the title compound (4).

### Synthetic Example 4

### Synthesis of 1,3-Di-O-acetyl-1,25-dihydroxy-16,23E-diene-cholecalciferol (5)

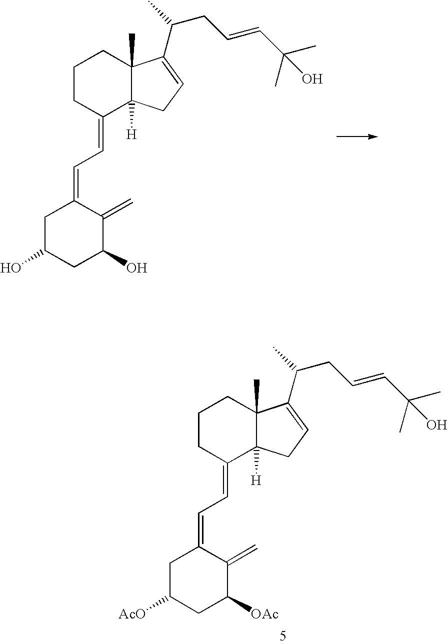

0.0468 g of 1,25-Dihydroxy-16,23E-diene-cholecalciferol was dissolved in 1.5 mL of pyridine. This solution was cooled in an ice bath then refrigerated overnight, diluted with 10 mL of water while still immersed in the ice bath, stirred for 10 min and transferred to a separatory funnel with the aid of 10 mL of water and 40 mL of ethyl acetate. The organic layer was washed with 4×20 mL of water, 10 mL of brine passed through a plug of sodium sulfate and evaporated. The light brown, oily residue was taken up in 1:9 ethyl acetate-hexane then flash chromatographed on a 10×130 mm column using 1:9 ethyl acetate-hexane as mobile phase for fractions 1-3 (20 mL fractions), 1:6 for fractions 6-8 and 1:4 ethyl acetate-hexane for fractions 9-17 (18 mL each). Fractions 11-14 contained the main band with Rf 0.09 (TLC 1:4). Those fractions were pooled and evaporated to a colorless oil, 0.0153 g. This material was taken up in methyl formate, filtered and evaporated, to give 0.014 g of the title compound (5).

### Synthetic Example 6

### Synthesis of 1,3-Di-O-acetyl-1,25-dihydroxy-16-ene-cholecalciferol (6)

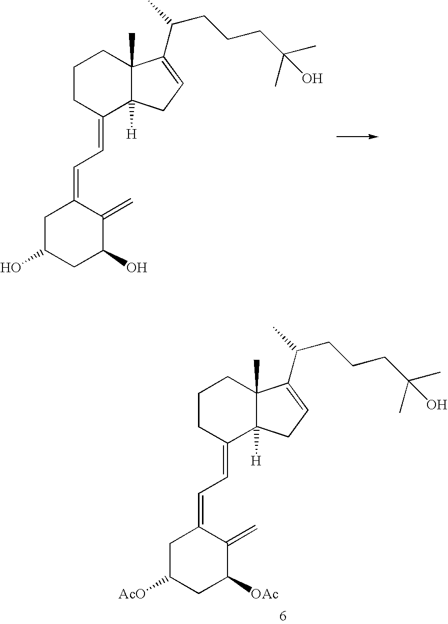

0.0774 g of 1,25-Dihydroxy-16-ene-cholecalciferol was dissolved in 1.5 mL of pyridine. This solution was cooled in an ice bath then 0.3 mL of acetic anhydride was added. The solution was stirred, refrigerated overnight then diluted with 1 mL of water, stirred for 1 h in the ice bath and diluted with 30 mL of ethyl acetate and 15 mL of water. The organic layer was washed with 4×15 mL of water, once with 5 mL of brine then dried (sodium sulfate) and evaporated. The light brown, oily residue was taken up in 1:9 ethyl acetate-hexane then flash chromatographed on a 10×130 mm column using 1:9 ethyl acetate-hexane as mobile phase for fraction 1 (20 mL fractions), 1:6 for fractions 2-7 and 1:4 ethyl acetate-hexane for fractions 8-13. Fractions 9-11 contained the main band with Rf 0.09 (TLC 1:4 ethyl acetate-hexane). Those fractions were pooled and evaporated to a colorless oil, 0.0354 g. This material was taken up in methyl formate, filtered and the solution evaporated, 0.027 g colorless film, the title compound (6).

### Synthetic Example 7

### Synthesis of 1,3,25-Tri-O-acetyl-1,25-dihydroxy-16-ene-23-yne-26,27-hexafluoro-cholecalciferol (7) and 1,3-Di-O-acetyl-1,25-dihydroxy-16-ene-23-yne-26,27-hexafluoro-cholecalciferol (8)

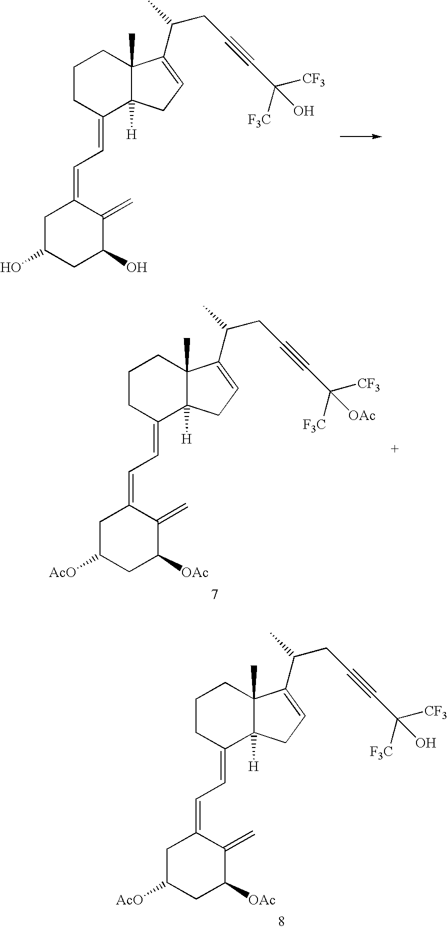

0.0291 g of 1,25-dihydroxy-16-ene-23-yne-26,27-hexafluoro-cholecalciferol was dissolved in 1.5 mL of pyridine. This solution was cooled in an ice bath then 0.25 mL of acetic anhydride was added. The solution was stirred for 20 min and kept in a freezer overnight. The cold solution was diluted with 15 mL of water, stirred for 10 min, and diluted with 30 mL of ethyl acetate. The organic layer was washed with 4×15 mL of water, once with 5 mL of brine then dried (sodium sulfate) and evaporated. The light brown, oily residue was taken up in 1:6 ethyl acetate-hexane then flash chromatographed on a 10×110 mm column using 1:6 ethyl acetate-hexane as mobile phase. Fractions 2-3 gave 72.3461−72.3285=0.0176 g. Evaporation of fractions 6-7 gave 0.0055 g. The residue of fractions 2-3 was taken up in methyl formate, filtered and evaporated to give 0.0107 g of the title triacetate (7). The residue of fractions 6-7 was taken up in methyl formate, filtered and evaporated to give 0.0049 g of diacetate (8).

### Synthetic Example 8

### Synthesis of 1,3-Di-O-acetyl-1,25-dihydroxy-16,23E-diene-25R,26-trifluoro-cholecalciferol (9)

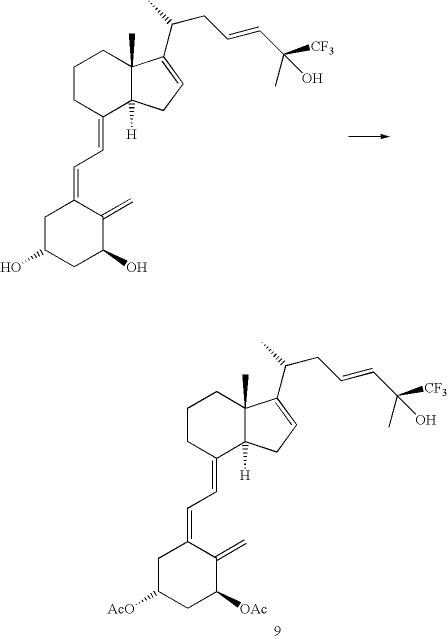

1.5 mL of 1,25-dihydroxy-16,23E-diene-25R,26-trifluoro-cholecalciferol was dissolved in 1.5 mL of pyridine, cooled to ice-bath temperature and 0.4 mL of acetic anhydride was added. The mixture was then refrigerated. After two days the mixture was diluted with 1 mL of water, stirred for 10 min in the ice bath then distributed between 10 mL of water and 30 mL of ethyl acetate. The organic layer was washed with 4×15 mL of water, once with 5 mL of brine then dried (sodium sulfate) and evaporated. The light brown, oily residue was taken up in 1:6 ethyl acetate-hexane then flash chromatographed on a 10×130 mm column using 1:6 ethyl acetate-hexane as mobile phase. Fractions 4-6 (TLC, 1:4) contained the main band (see TLC) These fractions were evaporated and gave 0.0726 g. This residue was taken up in methyl formate, filtered and evaporated, to give 0.0649 g of colorless foam, the title compound (9).

### Synthetic Example 8

### Synthesis of 1,3-Di-O-acetyl-1,25-dihydroxy-16-ene-19-nor-cholecalciferol (10) (“Compound C”)

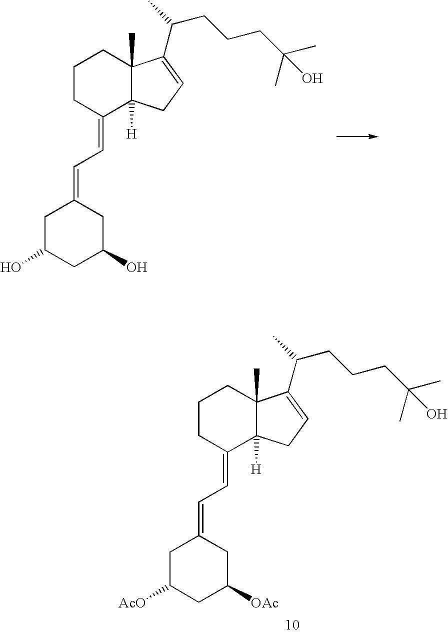

0.0535 g of 1,25-Dihydroxy-16-ene-19-nor-cholecalciferol was dissolved in 1.5 mL of pyridine, cooled to ice-bath temperature and 0.3 mL of acetic anhydride was added and the mixture was refrigerated overnight. The solution was diluted with 1 mL of water, stirred for 10 min in the ice bath then distributed between 10 mL of water and 30 mL of ethyl acetate. The organic layer was washed with 4×15 mL of water, once with 5 mL of brine then dried (sodium sulfate) and evaporated. The nearly colorless, oily residue was taken up in 1:6 ethyl acetate-hexane as mobile phase for fractions 1-6 then 1:4 ethyl acetate-hexane was used. Fractions 9-19 (TLC, 1:4 ethyl acetate-hexane, Rf 0.09, see below) were pooled, evaporated, to give 0.0306 g, which was taken up in methyl formate, filtered, then evaporated. It gave 0.0376 of the title compound (10).

### Synthetic Example 9

### Synthesis of 1,3-Di-O-Acetyl-1,25-dihydroxy-16-ene-23-yne-19-nor-cholecalciferol (11)

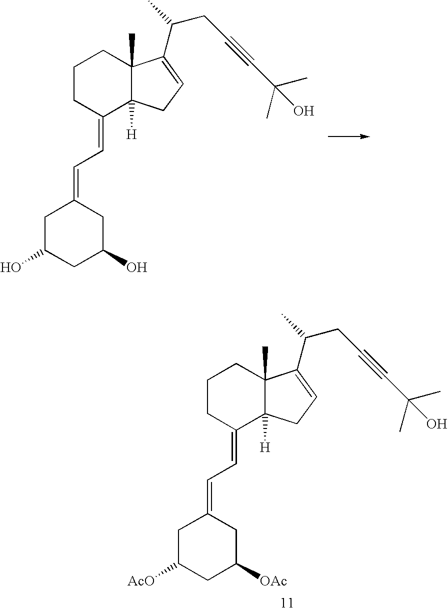

50 mg of 1,25-dihydroxy-16-ene-23-yne-19-nor-cholecalciferol was dissolved in 0.8 mL of pyridine, cooled to ice-bath temperature and 0.2 mL of acetic anhydride was added. The mixture was refrigerated for 3 days then diluted with 1 mL of water, stirred for 10 min in the ice bath and distributed between 5 mL of water and 20 mL of ethyl acetate. The organic layer was washed with 4×5 mL of water, once with 3 mL of brine then dried (sodium sulfate) and evaporated. The nearly colorless, oily residue was taken up in 1:6 ethyl acetate-hexane then flash chromatographed on a 15×120 mm column using 1:6 ethyl acetate-hexane as mobile phase for fractions 1-6, 1:4 for fractions 9-12, 1:3 for fractions 13-15 and 1:2 ethyl acetate-hexane for the remaining fractions. Fractions 11-16 (TLC, 1:4 ethyl acetate-hexane, Rf 0.09, see below) were pooled, evaporated 76.1487-76.1260=0.0227 g, taken up in methyl formate, filtered, then evaporated. It gave 0.0186 g of the title compound (11).

### Synthetic Example 10

### Synthesis of 1,3-Di-O-acetyl-1,25-dihydroxy-16-ene-23-yne-26,27-bishomo-19-nor-cholecalciferol (12)

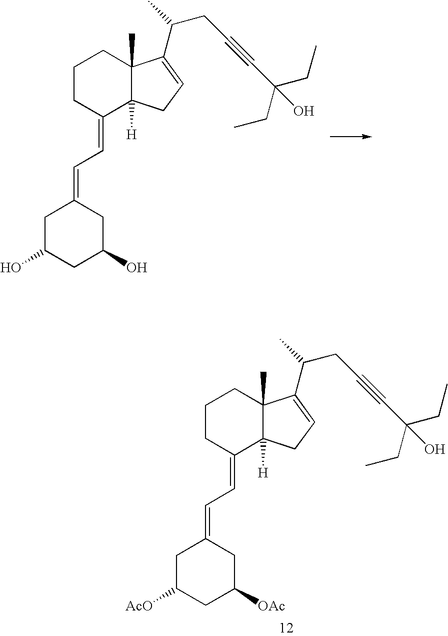

0.0726 g of 1,25-dihydroxy-16-ene-23-yne-26,27-bishomo-19-nor-cholecalciferol was dissolved in 0.8 mL of pyridine, cooled to ice-bath temperature and 0.2 mL of acetic anhydride was added. The solution was stirred in the ice-bath then refrigerated overnight. The solution was then diluted with 1 mL of water, stirred for 10 min in the ice bath and distributed between 10 mL of water and 25 mL of ethyl acetate. The organic layer was washed with 3×10 mL of water, once with 5 mL of saturated sodium hydrogen carbonate, once with 3 mL of brine then dried and evaporated, 33.5512−33.4654=0.0858 g of a tan oily residue that was flash-chromatographed on a 15×120 mm column using 1:6 as mobile phase. Fractions 7-11 (20 mL each) were pooled (TLC 1:4 ethyl acetate-hexane, Rf 0.14) and evaporated, 67.2834−67.2654=0.018 g. This residue was taken up in methyl formate, filtered and evaporated. It gave 0.0211 g of the title compound (12).

### Synthetic Example 11

### Synthesis of 1,3-Di-O-acetyl-1,25-dihydroxy-20-cyclopropyl-23-yne-19-nor-cholecalciferol (13)

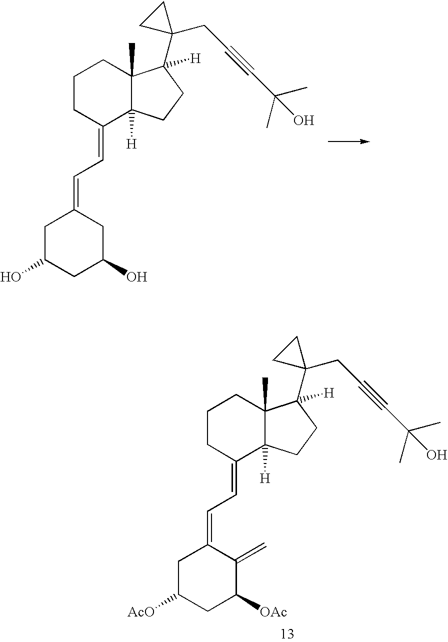

0.282 g of 1,25-Dihydroxy-20-cyclopropyl-23-yne-19-nor-cholecalciferol was dissolved in 0.8 mL of pyridine, cooled to ice-bath temperature and 0.2 mL of acetic anhydride was added and the mixture was refrigerated overnight, then diluted with 1 mL of water, stirred for 10 min in the ice bath and distributed between 5 mL of water and 20 mL of ethyl acetate. The organic layer was washed with 3×5 mL of water, once with 5 mL of saturated sodium hydrogen carbonate, once with 3 mL of brine then dried (sodium sulfate) and evaporated. The oily residue was taken up in 1:6 ethyl acetate-hexane then flash chromatographed on a 15×110 mm column using 1:6 ethyl acetate-hexane as mobile phase for fractions 1-4, 1:4 for fractions 5-12, 1:3 for fractions 13-15 ethyl acetate-hexane for the remaining fractions. Fractions 7-12 (TLC, 1:4 ethyl acetate-hexane, Rf 0.13) were pooled, evaporated, the residue taken up in methyl formate, filtered, then evaporated to give 0.023 g of the title compound (13).

### Synthetic Example 12

### Synthesis of 1,3,25-Tri-O-acetyl-1,25-dihydroxy-20-cyclopropyl-23-yne-26,27-hexafluoro-19-nor-cholecalciferol (14) and 1,3-Di-O-acetyl-1,25-dihydroxy-20-cyclopropyl-23-yne-26,27-hexafluoro-19-nor-cholecalciferol (15)

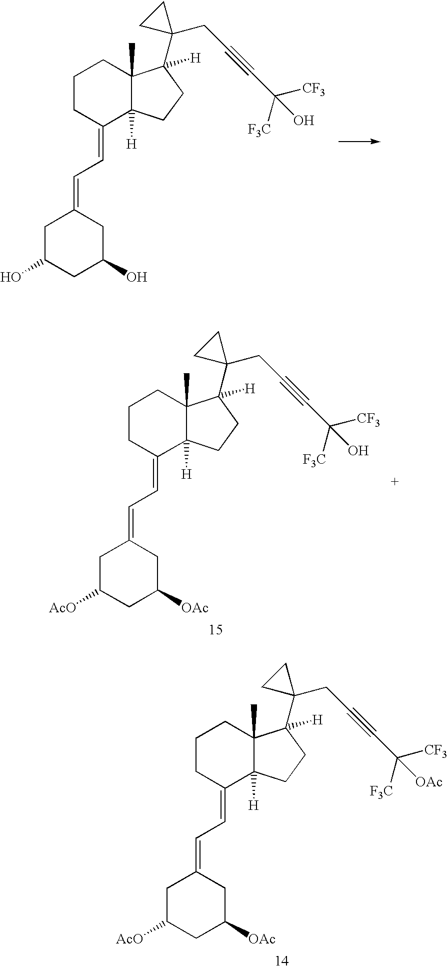

0.1503 g of 1,25-dihydroxy-20-cyclopropyl-23-yne-26,27-hexafluoro-19-nor-cholecalciferol was dissolved in 0.8 mL of pyridine, cooled to ice-bath temperature and 0.2 mL of acetic anhydride was added. The mixture was refrigerated overnight then diluted with 1 mL of water, stirred for 10 min in the ice bath and distributed between 5 mL of water and 20 mL of ethyl acetate. The organic layer was washed with 3×5 mL of water, once with 5 mL of saturated sodium hydrogen carbonate, once with 3 mL of brine then dried (sodium sulfate) and evaporated. The oily residue was taken up in 1:6 ethyl acetate-hexane then flash chromatographed on a 15×150 mm column using 1:6 ethyl acetate-hexane as mobile phase for fractions 1-5, 1:4 for the remaining fractions. Fractions 3-4 and 6-7 were pooled, evaporated, then taken up in methyl formate, filtered, and evaporated to give 0.0476 g of the title triacetate (14) and 0.04670 g of the title diacetate (15).

### Synthetic Example 13

### Synthesis of 1,3-Di-O-acetyl-1,25-dihydroxy-20-cyclopropyl-23-yne-cholecalciferol (16)

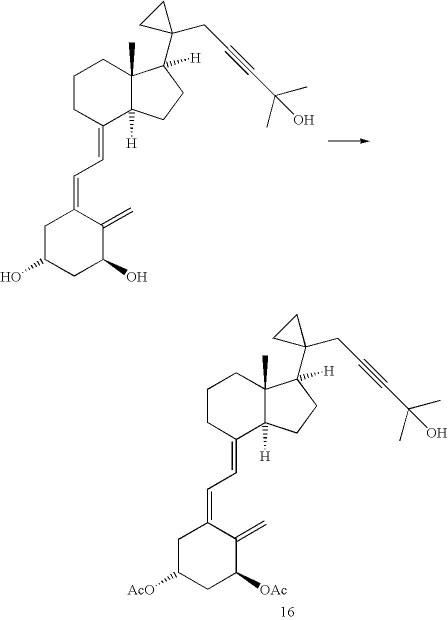

0.0369 g of 1,25-dihydroxy-20-cyclopropyl-23-yne-cholecalciferol was dissolved in 0.8 mL of pyridine, cooled to ice-bath temperature and 0.2 mL of acetic anhydride was added and the mixture was refrigerated overnight, then diluted with 1 mL of water, stirred for 10 min in the ice bath and distributed between 5 mL of water and 20 mL of ethyl acetate. The organic layer was washed with 3×5 mL of water, once with 5 mL of saturated sodium hydrogen carbonate, once with 3 mL of brine then dried (sodium sulfate) and evaporated. The oily residue was taken up in 1:6 ethyl acetate-hexane then flash-chromatographed on a 13×110 mm column using 1:6 ethyl acetate-hexane as mobile phase for fractions 1-7, 1:4 ethyl acetate-hexane for the remaining fractions. Fractions 9-11 (TLC, 1:4 ethyl acetate-hexane) were pooled, evaporated, taken up in methyl formate, filtered, then evaporated, to give 0.0099 g of the title compound (16).

### Synthetic Example 14

### Synthesis of 1,3-Di-O-acetyl-1,25-dihydroxy-20-cyclopropyl-23E-ene-26,27-hexafluoro-19-nor-cholecalciferol (17) (Compound E)

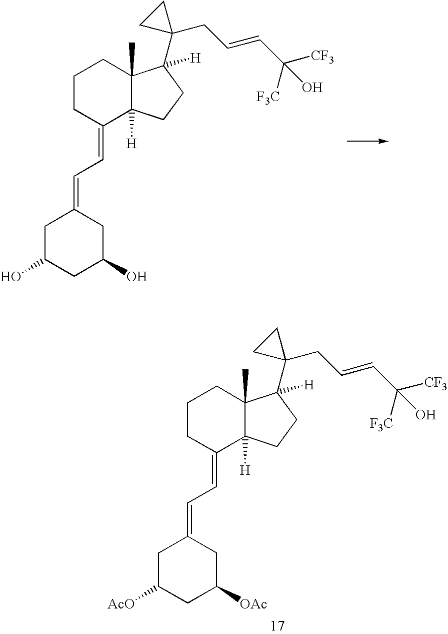

0.0328 g of 1,25-dihydroxy-20-cyclopropyl-23E-ene-26,27-hexafluoro-19-nor-cholecalciferol was dissolved in 0.8 mL of pyridine, cooled to ice-bath temperature and 0.2 mL of acetic anhydride was added. The solution was refrigerated overnight. The solution was then diluted with 1 mL of water, stirred for 10 min in the ice bath and distributed between 5 mL of water and 20 mL of ethyl acetate. (Extraction of the aqueous layer gave no phosphomolybdic acid-detectable material). The organic layer was washed with 3×5 mL of water, once with 5 mL of saturated sodium hydrogen carbonate, once with 3 mL of brine then dried (sodium sulfate) and evaporated, the residue shows Rf 0.25 as the only spot. The oily residue was taken up in 1:6 ethyl acetate-hexane then flash-chromato-graphed on a 13.5×110 mm column using 1:6 ethyl acetate-hexane as mobile phase for fractions 1-10. Fractions 4-9 were pooled and evaporated, the residue taken up in methyl formate, filtered, then evaporated to give 0.0316 g of the title compound (17).

### Synthetic Example 15

### Synthesis of 1,3-Di-O-acetyl-1,25-dihydroxy-20-cyclopropyl-23Z-ene-26,27-hexafluoro-19-nor-cholecalciferol (18)

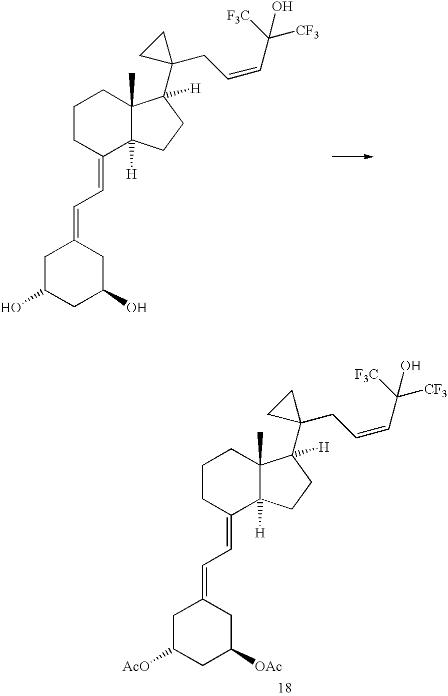

0.0429 g of 1,25-dihydroxy-20-cyclopropyl-23Z-ene-26,27-hexafluoro-19-nor-cholecalciferol was dissolved in 0.8 mL of pyridine, cooled to ice-bath temperature and 0.2 mL of acetic anhydride was added. The solution was refrigerated overnight. The solution was then diluted with 1 mL of water, stirred for 10 min in the ice bath and distributed between 7 mL of water and 25 mL of ethyl acetate. The organic layer was washed with 3×5 mL of water, once with 5 mL of saturated sodium hydrogen carbonate, once with 3 mL of brine then dried (sodium sulfate, TLC (1:4 ethyl acetate-hexane shows mostly one spot) and evaporated, flash-chromatographed on a 15×120 mm column using 1:6 as mobile phase. Fractions 3-6 (20 mL each) were pooled and evaporated. The residue was taken up in methyl formate, filtered and evaporated, to give 0.0411 g of the title compound (18).

### Synthetic Example 16

### Synthesis of 1,3-Di-O-acetyl-1,25-dihydroxy-20-cyclopropyl-cholecalciferol (19) (Compound F)

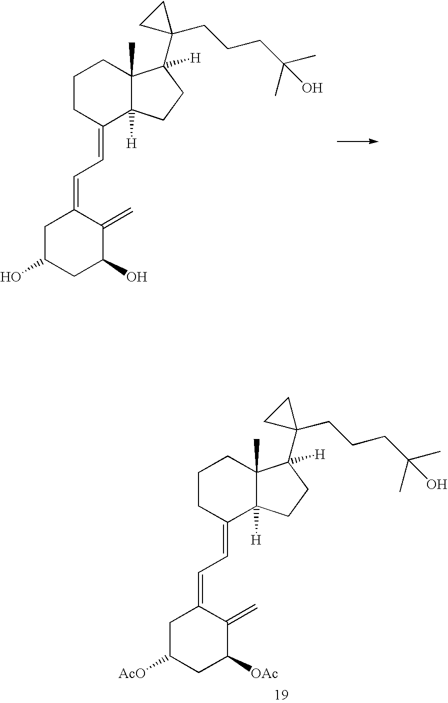

0.0797 g of 1,25-dihydroxy-20-cyclopropyl-cholecalciferol was dissolved in 0.8 mL of pyridine, cooled to ice-bath temperature and 0.2 mL of acetic anhydride was added. The solution was refrigerated overnight. The solution was then diluted with 1 mL of water, stirred for 10 min in the ice bath and distributed between 10 mL of water and 25 mL of ethyl acetate. The organic layer was washed with 3×10 mL of water, once with 5 mL of saturated sodium hydrogen carbonate, once with 3 mL of brine then dried and evaporated, to give 0.1061 g of a tan oily residue that was flash-chromatographed on a 15×120 mm column using 1:6 as mobile phase. Fractions 9-16 (20 mL each) were pooled (TLC 1:4 ethyl acetate-hexane, Rf 0.13) and evaporated. This residue was taken up in methyl formate, filtered and evaporated to give 0.0581 g of the title compound (19).

### Synthetic Example 17

### Synthesis of 1,3-Di-O-acetyl-1-alpha,25-dihydroxy-16-ene-20-cyclopropyl-19-nor-cholecalciferol (20)

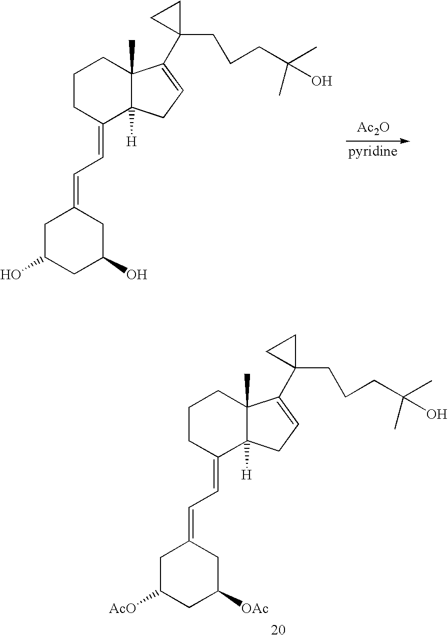

To the solution of 1-alpha,25-Dihydroxy-16-ene-20-cyclopropyl-19-nor-cholecalciferol (94 mg, 0.23 mmol) in pyridine (3 mL) at 0° C., acetic anhydride (0.5 mL, 5.3 mmol) was added. The mixture was stirred for 1 h, refrigerated for 15 h. and then was stirred for additional 8 h. Water (10 mL) was added and after stirring for 15 min. the reaction mixture was extracted with AcOEt:Hexane 1:1 (25 mL), washed with water (4×25 mL) and brine (20 mL), dried over Na₂SO₄. The residue (120 mg) after evaporation of the solvent was purified by FC (15 g, 30% AcOEt in hexane) to give the titled compound (20) (91 mg, 0.18 mmol, 80%). [α]³⁰_{D}=+14.4 c 0.34, EtOH; UV λmax (EtOH): 242 nm (ε 34349), 250 nm (ε 40458), 260 nm (ε 27545); ¹H NMR (CDCl₃): 6.25 (1H, d, J=11.1 Hz), 5.83 (1H, d, J=11.3 Hz), 5.35 (1H, m), 5.09 (2H, m), 2.82-1.98 (7H, m), 2.03 (3H, s), 1.98 (3H, s), 2.00-1.12 (15H, m), 1.18 (6H, s), 0.77 (3H, s), 0.80-0.36 (4H, m); ¹³C NMR (CDCl₃): 170.73 (0), 170.65 (0), 157.27 (0), 142.55 (0), 130.01 (0), 125.06 (1), 123.84 (1), 115.71, 71.32 (0), 70.24 (1), 69.99 (1), 59.68 (1), 50.40 (0), 44.08 (2), 41.40 (2), 38.37 (2), 35.96 (2), 35.80 (2), 32.93 (2), 29.48 (3), 29.31 (2), 28.71 (2), 23.71 (2), 22.50 (2), 21.56 (3), 21.51 (0), 21.44 (3), 18.01 (3), 12.93 (2), 10.53 (2); MS HRES Calculated for C₃₁H₄₆O₅ M+Na 521.3237, Observed M+Na 521.3233

### Synthetic Example 18

### Synthesis of 1,3-Di-O-acetyl-1-alpha,25-hydroxy-16-ene-20-cyclopropyl-cholecalciferol (21) pyridine

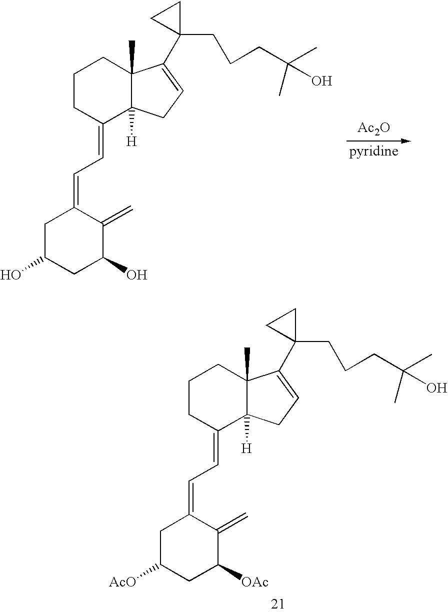

To the solution of 1-alpha,25-Dihydroxy-16-ene-20-cyclopropyl-cholecalciferol (100 mg, 0.23 mmol) in pyridine (3 mL) at 0° C., acetic anhydride (0.5 mL, 5.3 mmol) was added. The mixture was stirred for 2 h and then refrigerated for additional 15 h. Water (10 mL) was added and after stirring for 15 min. the reaction mixture was extracted with AcOEt: Hexane 1:1 (25 mL), washed with water (4×25 mL), brine (20 mL) and dried over Na₂SO₄. The residue (150 mg) after evaporation of the solvent was purified by FC (15 g, 30% AcOEt in hexane) to give the titled compound (21) (92 mg, 0.18 mmol, 78%). [α]³⁰_{D}=−14.9 c 0.37, EtOH; UV λmax (EtOH): 208 nm (ε 15949), 265 nm (ε 15745); ¹H NMR (CDCl₃): 6.34 (1H, d, J=11.3 Hz), 5.99 (1H, d, J=11.3 Hz), 5.47 (1H, m), 5.33 (1H, m), 5.31 (1H, s), 5.18 (1H, m), 5.04 (1H, s), 2.78 (1H, m), 2.64 (1H, m), 2.40-1.10 (18H, m), 2.05 (3H, s), 2.01 (3H, s), 1.18 (6H, s), 0.76 (3H, s), 0.66-0.24 (4H, m); ¹³C NMR (CDCl₃): 170.76 (0), 170.22 (0), 157.18 (0), 143.02 (0), 142.40 (0), 131.94 (0), 125.31 (1), 125.10 (1), 117.40 (1), 115.22 (2), 72.97 (1), 71.32 (0), 69.65 (1), 59.71 (1), 50.57 (0), 44.07 (2), 41.73 (2), 38.36 (2), 37.10 (2), 35.80 (2), 29.45 (3), 29.35 (2), 29.25 (3), 28.92 (2), 23.80 (2), 22.48 (2), 21.55 (3), 21.50 (3), 21.35 (0), 17.90 (3), 12.92 (2), 10.54 (2); MS HRES Calculated for C₃₂H₄₆O₅ M+Na 533.3237, Observed M+Na 533.3236

### Synthetic Example 19

### Synthesis of 1,3-Di-O-acetyl-1,25-dihydroxy-23-yne-cholecalciferol (22)

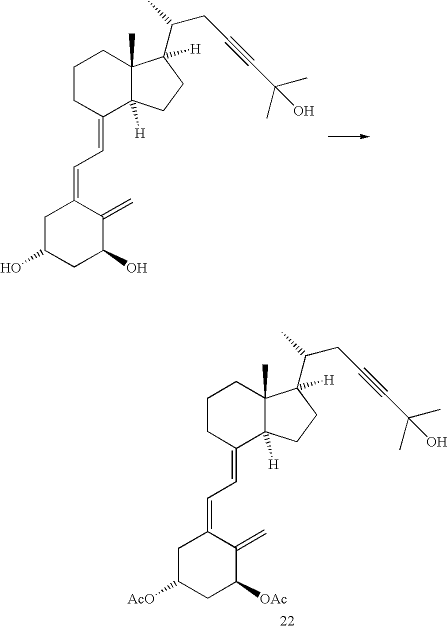

0.2007 g of (0.486 mmol) was dissolved in 2 mL of pyridine. This solution was cooled in an ice bath and 0.6 mL of acetic anhydride was added. The solution was kept in an ice bath for 45 h then diluted with 10 mL of water, stirred for 10 min and equilibrated with 10 mL of water and 40 mL of ethyl acetate. The organic layer was washed with 4×20 mL of water, 10 mL of brine, dried (sodium sulfate) and evaporated. The brown, oily residue was flash chromatographed using 1:19, 1:9, and 1:4 ethyl acetate-hexane as stepwise gradient. The main band with Rf 0.16 (TLC 1:4 acetate-hexane) was evaporated to give 1,3-di-O-acetyl-1,25-dihydroxy-23-yne-cholecalciferol (22) a colorless foam, 0.0939

### Synthetic Example 20

### Synthesis of (3aR,4S,7aR)-7a-Methyl-1-[1-(4-hydroxy-4-methyl-pent-2-ynyl)-cyclopropyl]-3a,4,5,6,7,7a-hexahydro-3H-inden-4-ol

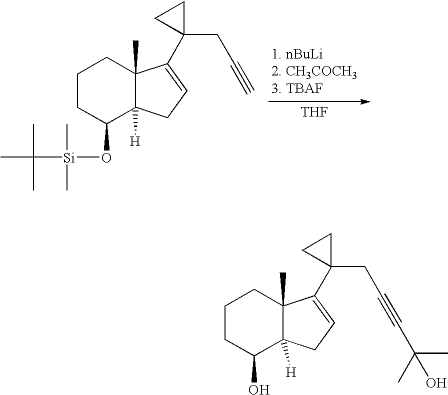

To a stirred solution of (3aR,4S,7aR)-1-{1-[4-(tert-Butyl-dimethyl-silanyloxy)-7a-methyl-3a,4,5,6,7,7a-hexahydro-3H-inden-1-yl])-cyclopropyl}-ethynyl (1.0 g, 2.90 mmol) in tetrahydrofurane (15 mL) at −78° C. was added n-BuLi (2.72 mL, 4.35 mmol, 1.6M in hexane). After stirring at −78° C. for 1 h., acetone (2.5 mL, 34.6 mmol) was added and the stirring was continued for 2.5 h. NH₄Cl_{aq} was added (15 mL) and the mixture was stirred for 15 min at room temperature then extracted with AcOEt (2×50 mL). The combined extracts were washed with brine (50 mL) and dried over Na₂SO₄. The residue after evaporation of the solvent (2.4 g) was purified by FC (50 g, 100% AcOEt in hexane) to give (3aR,4S,7aR)-5-{1-[4-(tert-Butyl-dimethyl-silanyloxy)-7a-methyl-3a,4,5,6,7,7a-hexahydro-3H-inden-1-yl]-cyclopropyl}-2-methyl-pent-3-yn-2-ol (1.05 g, 2.61 mmol) which was treated with tetrabutylammonium fluoride (6 mL, 6 mmol, 1.0M in THF) and stirred at 65-75° C. for 48 h. The mixture was diluted with AcOEt (25 mL) and washed with water (5×25 mL), brine (25 mL). The combined aqueous washes were extracted with AcOEt (25 mL) and the combined organic extracts were dried over Na₂SO₄. The residue after evaporation of the solvent (1.1 g) was purified by FC (50 g, 20% AcOEt in hexane) to give the titled compound (0.75 g, 2.59 mmol, 90%). [α]³⁰_{D}=+2.7 c 0.75, CHCl₃ ¹H NMR (CDCl₃): 5.50 (1H, m), 4.18 (1H, m), 2.40 (2H, s), 2.35-1.16 (11H, m), 1.48 (6H, s), 1.20 (3H, s), 0.76-0.50 (4H, m); ¹³C NMR (CDCl₃): 156.39, 125.26, 86.39, 80.19, 69.21, 65.16, 55.14, 46.94, 35.79, 33.60, 31.67, 29.91, 27.22, 19.32, 19.19, 17.73, 10.94, 10.37; MS HREI Calculated for C₂₂H₂₈O₂ M+ 288.2089, Observed M+ 288.2091.

### Synthetic Example 21

### Synthesis of (3aR,4S,7aR)-7a-Methyl-1-[1-(4-hydroxy-4-methyl-pent-2Z-enyl)-cyclopropyl]-3a,4,5,6,7,7a-hexahydro-3H-inden-4-ol

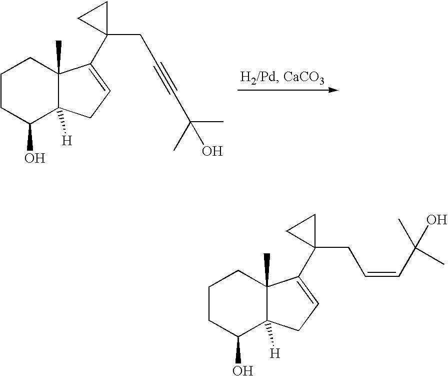

The mixture of (3aR,4S,7aR)-7a-Methyl-1-[1-(-4-hydroxy-4-methyl-pent-2-ynyl)-cyclopropyl]-3a,4,5,6,7,7a-hexahydro-3H-inden-4-ol (0.72 g, 2.50 mmol), ethyl acetate (10 mL), hexane (24 mL), absolute ethanol (0.9 mL), quinoline (47 L) and Lindlar catalyst (156 mg, 5% Pd on CaCO₃) was hydrogenated at room temperature for 2 h. The reaction mixture was filtered through a celite pad and the pad was washed with AcOEt. The filtrates and the washes were combined and washed with 1M HCl, NaHCO₃ and brine. After drying over Na₂SO₄ the solvent was evaporated and the residue (0.79 g) was purified by FC (45 g, 20% AcOEt in hexane) to give the titled compound (640 mg, 2.2 mmol, 88%).

### Synthetic Example 22

### Synthesis of (3aR,4S,7aR)-7a-Methyl-1-[1-(4-hydroxy-4-methyl-pentyl)-cyclopropyl]-3a,4,5,6,7,7a-hexahydro-3H-inden-4-ol

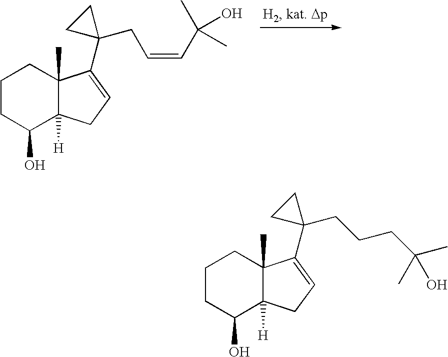

The mixture of (3aR,4S,7aR)-7a-Methyl-1-[1-(4-hydroxy-4-methyl-pent-2Z-enyl)-cyclopropyl]-3a,4,5,6,7,7a-hexahydro-3H-inden-4-ol (100 mg, 0.34 mmol), 1,4-bis(diphenyl-phosphino)butane 1,5 cyclooctadiene rhodium tetrafluoroborate (25 mg, 0.034 mmol), dichloromethane (5 mL) and one drop of mercury was hydrogenated using Paar apparatus at room temperature and 50 p.s.i. pressure for 3 h. The reaction mixture was filtered through Celite pad, which was then washed with ethyl acetate. The combine filtrates and washes were evaporated to dryness (110 mg) and purified by FC (10 g, 20% AcOEt in hexane) to give the titled compound (75 mg, 0.26 mmol, 75%). [α]³⁰_{D}=−8.5 c 0.65, CHCl₃. ¹H NMR (CDCl₃): 5.37 (1H, m,); 4.14 (1H, m), 2.37-1.16 (17H, m), 1.19 (6H, s), 1.18 (3H, s), 0.66-0.24 (4H, m); MS HREI Calculated for C₁₉H₃₂O₂ M+H 292.2402. Observed M+H 292.2404.

### Synthetic Example 23

### Synthesis of (3aR,7aR)-7a-Methyl-1-[1-(4-methyl-4-trimethylsilanyloxy-pentyl)-cyclopropyl]-3a,4,5,6,7,7a-hexahydro-3H-inden-4-one

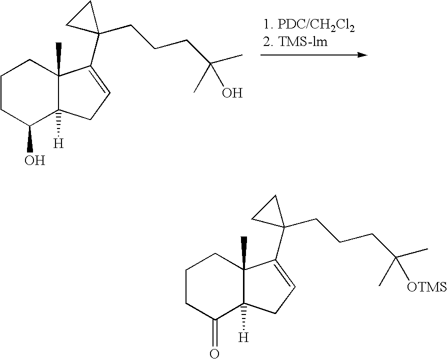

To a stirred suspension of (3aR,4S,7aR)-7a-Methyl-1-[1-(4-hydroxy-4-methyl-pentenyl)-cyclopropyl]-3a,4,5,6,7,7a-hexahydro-3H-inden-4-ol (440 mg, 1.50 mmol) and Celite (2.0 g) in dichloromethane (10 mL) at room temperature wad added pyridinium dichromate (1.13 g, 3.0 mmol). The resulting mixture was stirred for 5 h filtered through silica gel (10 g), and then silica gel pad was washed with 20% AcOEt in hexane. The combined filtrate and washes were evaporated, to give a crude (3aR,7aR)-7a-Methyl-1-[1-(4-hydroxy-4-methyl-pentenyl)-cyclopropyl]-3a,4,5,6,7,7a-hexahydro-3H-inden-4-one (426 mg, 1.47 mmol, 98%). To a stirred solution of (3aR,7aR)-7a-Methyl-1-[1-(4-hydroxy-4-methyl-pentenyl)-cyclopropyl]-3a,4,5,6,7,7a-hexahydro-3H-inden-4-one (424 mg, 1.47 mmol) in dichloromethane (10 mL) at room temperature was added trimethylsilyl-imidazole (0.44 mL, 3.0 mmol). The resulting mixture was stirred for 1.0 h filtered through silica gel (10 g) and the silica gel pad was washed with 10% AcOEt in hexane. Combined filtered and washes were evaporated to give the titled compound (460 mg, 1.27 mmol, 86%). [α]²⁹_{D}=−9.9 c 0.55, CHCl₃. ¹H NMR (CDCl₃): 5.33 (1H, dd, J=3.2, 1.5 Hz), 2.81 (1H, dd, J=10.7, 6.2 Hz), 2.44 (1H, ddd, J=15.6, 10.7, 1.5 Hz), 2.30-1.15 (13H, m) overlapping 2.03 (ddd, J=15.8, 6.4, 3.2 Hz), 1.18 (6H, s), 0.92 (3H, s), 0.66-0.28 (4H, m), 0.08 (9H, s); ¹³C NMR (CDCl₃): 211.08 (0), 155.32 (0), 124.77 (1), 73.98 (0), 64.32 (1), 53.91 (0), 44.70 (2), 40.45 (2), 38.12 (2), 34.70 (2), 29.86 (3), 29.80 (3), 26.80 (2), 24.07 (2), 22.28 (2), 21.24 (0), 18.35 (3), 12.60 (2), 10.64 (2), 2.63 (3); MS HRES Calculated for C₂₂H₃₈O₂Si M+ 362.2641. Observed M+ 362.2648.

### Synthetic Example 24

### Synthesis of (3aR,7aR)-7a-Methyl-1-[1-(4-methyl-4-trimethylsilanyloxy-pent-2-ynyl)-cyclopropyl]-3a,4,5,6,7,7a-hexahydro-3H-inden-4-one

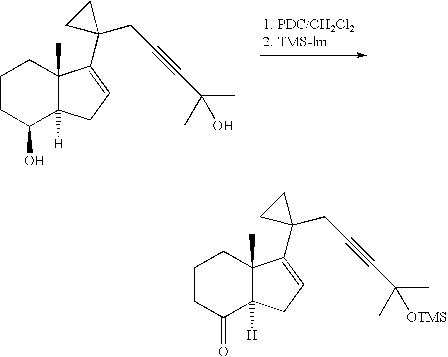

To a stirred suspension of (3aR,4S,7aR)-7a-Methyl-1-[1-(4-hydroxy-4-methyl-pent-2-ynyll)-cyclopropyl]-3a,4,5,6,7,7a-hexahydro-3H-inden-4-ol (381 mg, 1.32 mmol) and Celite (2.0 g) in dichloromethane (10 mL) at room temperature wad added pyridinium dichromate (1.0 g, 2.65 mmol). The resulting mixture was stirred for 1.5 h filtered through silica gel (10 g), and then silica gel pad was washed with 20% AcOEt in hexane. The combined filtrate and washes were evaporated, to give a crude (3aR,7aR)-7a-Methyl-1-[1-(4-hydroxy-4-methyl-pent-2-ynyll)-cyclopropyl]-3a,4,5,6,7,7a-hexahydro-3H-inden-4-one (360 mg, 1.26 mmol, 95%). To a stirred solution of (3aR,7aR)-7a-Methyl-1-[1-(4-hydroxy-4-methyl-pent-2-ynyll)-cyclopropyl]-3a,4,5,6,7,7a-hexahydro-3H-inden-4-one (360 mg, 1.26 mmol) in dichloromethane (10 mL) at room temperature was added trimethylsilyl-imidazole (0.25 mL, 1.7 mmol). The resulting mixture was stirred for 0.5 h filtered through silica gel (10 g) and the silica gel pad was washed with 5% AcOEt in hexane. Combined filtered and washes were evaporated to give the titled compound (382 mg, 1.07 mmol, 81%).

### Synthetic Example 25

### Synthesis of 1-alpha,25-Dihydroxy-16-ene-20-cyclopropyl-23,24-yne-cholecalciferol (23)

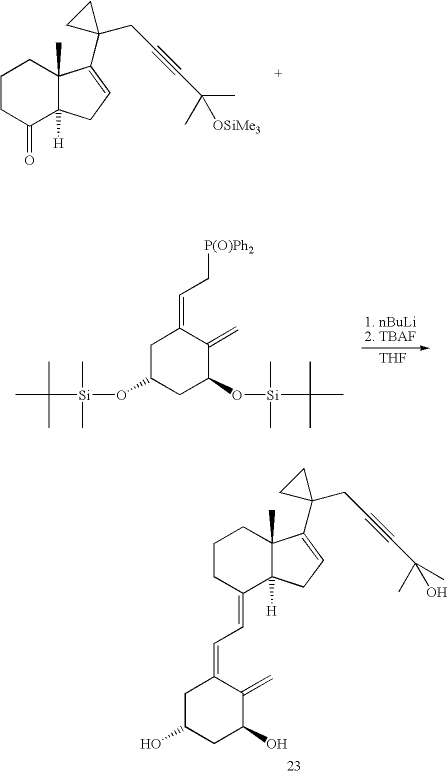

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (513 mg, 0.88 mmol) in tetrahydrofurane (6 mL) at −78° C. was added n-BuLi (0.55 mL, 0.88 mmol). The resulting mixture was stirred for 15 min and solution of (3aR,7aR)-7a-Methyl-1-[1-(4-methyl-4-trimethylsilanyloxy-pent-2-ynyll)-cyclopropyl]-3a,4,5,6,7,7a-hexahydro-3H-inden-4-one (179 mg, 0.50 mmol, in tetrahydrofurane (2 mL) was added dropwise. The reaction mixture was stirred at −72° C. for 3.5 h diluted with hexane (25 mL) washed brine (30 mL) and dried over Na₂SO₄. The residue (716 mg) after evaporation of the solvent was purified by FC (15 g, 5% AcOEt in hexane) to give 1-alpha,3-beta-Di(tert-Butyl-dimethyl-silanyloxy)-25-trimethylsilanyloxy-16-ene-20-cyclopropyl-23,24-yne-cholecalciferol (324 mg, 045 mmol). To the 1-alpha,3-beta-Di(tert-Butyl-dimethyl-silanyloxy)-25-trimethylsilanyloxy-16-ene-20-cyclopropyl-23,24-yne-cholecalciferol (322 mg, 0.45 mmol) tetrabutylammonium fluoride (4 mL, 4 mmol, 1M solution in THF) was added, at room temperature. The mixture was stirred for 18 h diluted with AcOEt (25 mL) and washed with water (5×20 mL), brine (20 mL) and dried over Na₂SO₄. The residue (280 mg) after evaporation of the solvent was purified by FC (10 g, 50% AcOEt in hexane and AcOEt) to give the titled compound (23) (172 mg, 0.41 mmol, 82%). [α]³¹_{D}=+32.4 c 0.50, MeOH. UV λmax (EtOH): 261 nm (ε 11930); ¹H NMR (CDCl₃): 6.36 (1H, d, J=11.3 Hz), 6.09 (1H, d, J=1.3 Hz), 5.45 (1H, m), 5.33 (1H, m), 5.01 (1H, s), 4.45 (1H, m), 4.22 (1H, m), 2.80 (1H, m), 2.60 (1H, m), 2.50-1.10 (16H, m), 1.45 (6H, s), 0.81 (3H, s), 0.72-0.50 (4H, m); MS HRES Calculated for C₂₈H₃₈O₃ M+ 422.2821. Observed M+ 422.2854.

### Synthetic Example 26

### Synthesis of 1-alpha,25-Dihydroxy-16-ene-20-cyclopropyl-23,24-yne-19-nor-cholecalciferol (24)

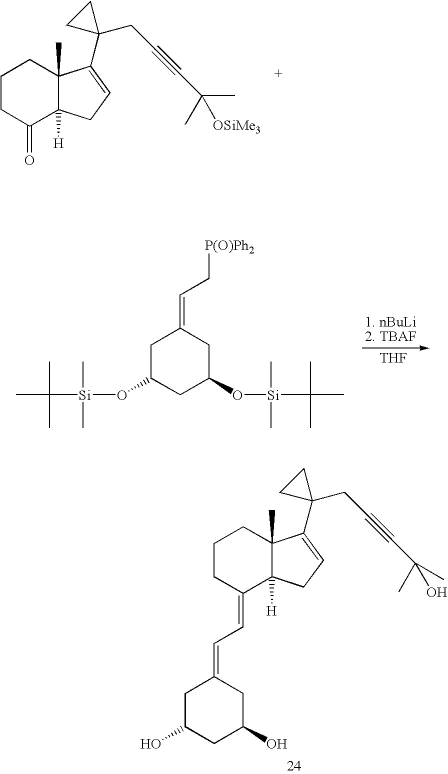

To a stirred solution of a (1R,3R)-1,3-bis-((tert-butyldimethyl)silanyloxy)-5-[2-(diphenylphosphinoyl)ethylidene]-cyclohexane (674 mg, 1.18 mmol) in tetrahydrofurane (8 mL) at −78° C. was added n-BuLi (0.74 mL, 1.18 mmol). The resulting mixture was stirred for 15 min and solution of (3aR,7aR)-7a-Methyl-1-[1-(4-methyl-4-trimethylsilanyloxy-pent-2-ynyl)-cyclopropyl]-3a,4,5,6,7,7a-hexahydro-3H-inden-4-one (235 mg, 0.66 mmol, in tetrahydrofurane (3 mL) was added dropwise. The reaction mixture was stirred at −72° C. for 3.5 h diluted with hexane (25 mL) washed brine (30 mL) and dried over Na₂SO₄. The residue (850 mg) after evaporation of the solvent was purified by FC (15 g, 5% AcOEt in hexane) to give 1-alpha,3-beta-Di(tert-Butyl-dimethyl-silanyloxy)-25-trimethylsilanyloxy-16-ene-20-cyclopropyl-23,24-yne-19-nor-cholecalciferol (330 mg, 0.46 mmol). To the 1-alpha,3-beta-Di(tert-Butyl-dimethyl-silanyloxy)-25-trimethylsilanyloxy-16-ene-20-cyclopropyl-23,24-yne-19-nor-cholecalciferol (328 mg, 0.46 mmol) tetrabutylammonium fluoride (5 mL, 5 mmol, 1M solution in THF) was added, at room temperature. The mixture was stirred for 62 h diluted with AcOEt (25 mL) and washed with water (5×20 mL), brine (20 mL) and dried over Na₂SO₄. The residue (410 mg) after evaporation of the solvent was purified by FC (10 g, 50% AcOEt in hexane and AcOEt) to give the titled compound (24) (183 mg, 0.45 mmol, 68%). [[α]²⁹_{D}=+72.1 c 0.58, MeOH. UV λmax (EtOH): 242 nm (ε 29286), . . . 251 nm (ε 34518), 260 nm (ε 23875); ¹H NMR (CDCl₃): 6.30 (1H, d, J=11.3 Hz), 5.94 (1H, d, J=11.3 Hz), 5.48 (1H, m), 4.14 (1H, m), 4.07 (1H, m), 2.78 (2H, m), 2.52-1.10 (18H, m), 1.49 (6H, s), 0.81 (3H, s), 0.72-0.50 (4H, m); MS HRES Calculated for C₂₇H₃₈O₃ M+ 410.2821. Observed M+ 410.2823.

### Synthetic Example 27

### Synthesis of (3aR,4S,7aR)-7a-Methyl-1-[1-(5,5,5-trifluoro-4-hydroxy-4-trifluoromethyl-pent-2-ynyl)-cyclopropyl]-3a,4,5,6,7,7a-hexahydro-3H-inden-4-ol

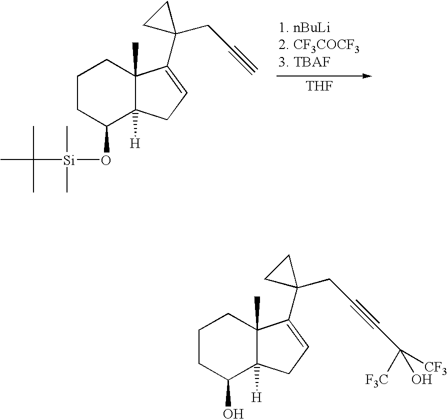

To a stirred solution of (3aR,4S,7aR)-1-{1-[4-(tert-Butyl-dimethyl-silanyloxy)-7a-methyl-3a,4,5,6,7,7a-hexahydro-3H-inden-1-yl])-cyclopropyl}-ethynyl (1.95 g, 5.66 mmol) in tetrahydrofurane (35 mL) at −78° C. was added n-BuLi (4.3 mL, 6.88 mmol, 1.6M in hexane). After stirring at −78° C. for 1 h., hexafluoroacetone (six drops from the cooling finger) was added and the stirring was continued for 1 h. NH₄Cl_{aq} was added (10 mL) and the mixture was allowed to warm to room temperature. The reaction mixture was diluted with brine (100 mL) and extracted with hexane (2×125 mL). The combined extracts were dried over Na₂SO₄. The residue after evaporation of the solvent (8.2 g) was purified by FC (150 g, 10% AcOEt in hexane) to give (3aR,4S,7aR)-5-{1-[4-(tert-Butyl-dimethyl-silanyloxy)-7a-methyl-3a,4,5,6,7,7a-hexahydro-3H-inden-1-yl]-cyclopropyl}-1,1,1-trifluoro-2-trifluoromethyl-pent-3-yn-2-ol (2.73 g, 5.35 mmol) which was treated with tetrabutylammonium fluoride (20 mL, 20 mmol, 1.0M in THF) and stirred at 65-75° C. for 30 h. The mixture was diluted with AcOEt (150 mL) and washed with water (5×150 mL), brine (150 mL). The combined aqueous washes were extracted with AcOEt (150 mL) and the combined organic extracts were dried over Na₂SO₄. The residue after evaporation of the solvent (3.2 g) was purified by FC (150 g, 20% AcOEt in hexane) to give the titled compound (2.05 g, 5.17 mmol, 97%). [α]²⁸_{D}=+6.0 c 0.47, CHCl_{3.1}H NMR (CDCl₃): 5.50 (1H, br. s), 4.16 (1H, br. s), 3.91 (1H, s), 2.48 (1H, part A of the AB quartet, J=17.5 Hz), 2.43 (1H, part B of the AB quartet, J=17.5 Hz), 2.27 (1H, m), 2.00-1.40 (9H, m), 1.18 (3H, s), 0.8-0.5 (4H, m); ¹³C NMR (CDCl₃): 155.26 (0), 126.68 (1), 121.32 (0, q, J=284 Hz), 90.24 (0), 71.44 (0, sep. J=34 Hz), 70.54 (O), 69.57 (1), 55.17 (1), 47.17 (0), 36.05 (2), 33.63 (2), 30.10 (2), 27.94 (2), 19.50 (3), 19.27 (0), 17.90 (2), 11.56 (2), 11.21 (2); MS HREI Calculated for C₁₉H₂₂O₂F₆ M+ 396.1524. Observed M+ 396.1513.

### Synthetic Example 28

### Synthesis of (3aR,7aR)-7a-Methyl-1-[1-(5,5,5-trifluoro-4-trifluoromethyl-4-hydroxy-pen-2-ynyl)-cyclopropyl]-3a,4,5,6,7,7a-hexahydro-3H-inden-4-one

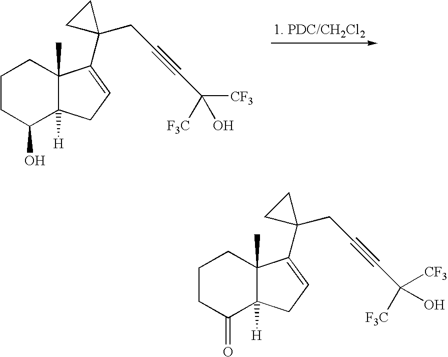

To a stirred suspension of (3aR,4S,7aR)-7a-Methyl-1-[1-(5,5,5-trifluoro-4-hydroxy-4-trifluoromethyl-pent-2-ynyl)-cyclopropyl]-3a,4,5,6,7,7a-hexahydro-3H-inden-4-ol (504 mg, 1.27 mmol) and Celite (1.5 g) in dichloromethane (12 mL) at room temperature wad added pyridinium dichromate (0.98 g, 2.6 mmol). The resulting mixture was stirred for 2.5 h filtered through silica gel (5 g), and then silica gel pad was washed with 20% AcOEt in hexane. The combined filtrate and washes were evaporated, to give a titled compound (424 mg, 1.08 mmol, 85%). [α]²⁸_{D}=+3.1 c 0.55, CHCl₃. ¹H NMR (CDCl₃): 5.46 (1H, br. s), 3.537 (1H, s), 2.81 (1H, dd, J=10.7, 6.5 Hz), 2.49-1.76 (10H, m), 0.90 (3H, s), 0.77-0.53 (4H, m); MS HREI Calculated for C₁₉H₂₀O₂F₆ M+H 395.1440. Observed M+H 395.1443.

### Synthetic Example 29

### Synthesis of 1-alpha,25-Dihydroxy-16-ene-20-cyclopropyl-23,24-yne-26,27-hexafluoro-19-nor-cholecalciferol (25)

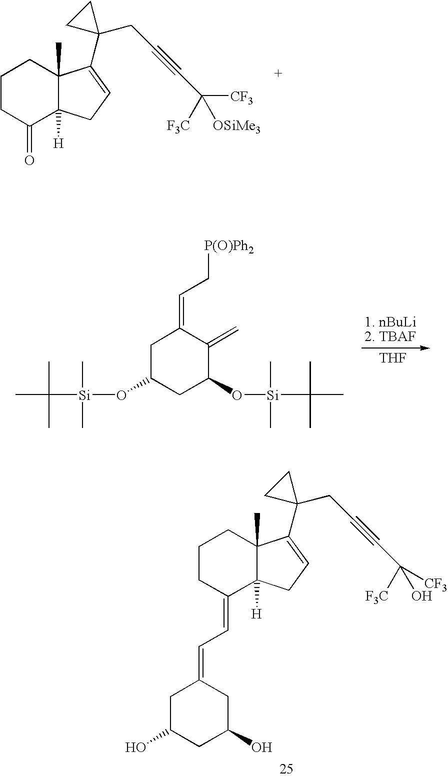

To a stirred solution of a (1R,3R)-1,3-bis-((tert-butyldimethyl)silanyloxy)-5-[2-(diphenylphosphinoyl)ethylidene]-cyclohexane (900 mg, 1.58 mmol) in tetrahydrofurane (8 mL) at −78° C. was added n-BuLi (1.0 mL, 1.6 mmol). The resulting mixture was stirred for 15 min and solution of (3aR,7aR)-7a-Methyl-1-[1-(5,5,5-trifluoro-4-trifluoromethyl-4-hydroxy-pen-2-ynyl)-cyclopropyl]-3a,4,5,6,7,7a-hexahydro-3H-inden-4-one (200 mg, 0.51 mmol, in tetrahydrofurane (3 mL) was added dropwise. The reaction mixture was stirred at −72° C. for 3.5 h diluted with hexane (25 mL) washed brine (30 mL) and dried over Na₂SO₄. The residue (850 mg) after evaporation of the solvent was purified by FC (20 g, 10% AcOEt in hexane) to give 1-alpha,3-beta-Di(tert-Butyl-dimethyl-silanyloxy)-25-hydroxy-16-ene-20-cyclopropyl-23,24-yne-26,27-hexafluoro-19-nor-cholecalciferol (327 mg, 0.44 mmol, 86%). To the 1-alpha,3-beta-Di(tert-Butyl-dimethyl-silanyloxy)-25-hydroxy-16-ene-20-cyclopropyl-23,24-yne-26,27-hexafluoro-19-nor-cholecalciferol (327 mg, 0.44 mmol). Tetrabutylammonium fluoride (4 mL, 4 mmol, 1M solution in THF) was added, at room temperature. The mixture was stirred for 24 h. diluted with AcOEt (25 mL) and washed with water (5×20 mL), brine (20 mL) and dried over Na₂SO₄. The residue (250 mg) after evaporation of the solvent was purified by FC (10 g, 50% AcOEt in hexane and AcOEt) to give the titled compound (25) (183 mg, 0.45 mmol, 68%). [α]³⁰_{D}=+73.3 c 0.51, EtOH. UV λmax (EtOH): 243 nm (ε 2938) 4 251 nm (ε 34973), 260 nm (ε 23924); ¹H NMR (CDCl₃): 6.29 (1H, d, J=11.1 Hz), 5.93 (1H, d, J=11.1 Hz), 5.50 (1H, m), 4.12 (1H, m), 4.05 (1H, m), 2.76 (2H, m), 2.55-1.52 (18H, m), 0.80 (3H, s), 0.80-0.49 (4H, m); ¹³C NMR (CDCl₃): 155.24 (0), 141.78 (0), 131.28 (0), 126.23 (1), 123.65 (1), 121.09 (0, q, J=285 Hz), 115.67 (1), 89.63 (0), 70.42 (0), 67.48 (1), 67.29 (1), 59.19 (1), 49.87 (0), 44.49 (2), 41.98 (2), 37.14 (2), 35.76 (2), 29.22 (2), 28.47 (2), 27.57 (2), 23.46 (2), 19.32 (0), 17.97 (3), 11.89 (2), 10.18 (2); MS HRES Calculated for C₂₇H₃₂O₃F₆ M+H 519.2329. Observed M+H 519.2325.

### Synthetic Example 30

### Synthesis of 1-alpha,25-Dihydroxy-16-ene-20-cyclopropyl-23,24-yne-26,27 hexafluoro-cholecalciferol (26)

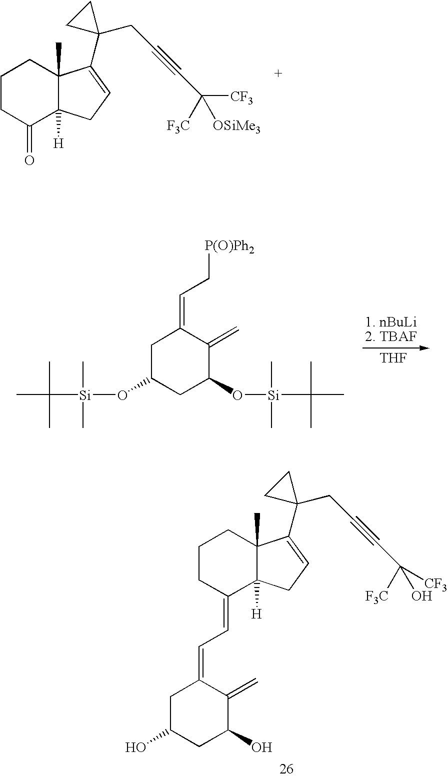

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (921 mg, 1.58 mmol) in tetrahydrofurane (8 mL) at −78° C. was added n-BuLi (1.0 mL, 1.6 mmol). The resulting mixture was stirred for 15 min and solution of (3aR,7aR)-7a-Methyl-1-[1-(5,5,5-trifluoro-4-trifluoromethyl-4-hydroxy-pen-2-ynyl)-cyclopropyl]-3a,4,5,6,7,7a-hexahydro-3H-inden-4-one (197 mg, 0.50 mmol, in tetrahydrofurane (2 mL) was added dropwise. The reaction mixture was stirred at −72° C. for 3.5 h diluted with hexane (25 mL) washed brine (30 mL) and dried over Na₂SO₄. The residue (876 mg) after evaporation of the solvent was purified by FC (20 g, 105% AcOEt in hexane) to give 1-alpha,3-beta-Di(tert-Butyl-dimethyl-silanyloxy)-25-hydroxy-16-ene-20-cyclopropyl-23,24-yne-26,27-hexafluoro-cholecalciferol (356 mg, 0.47 mmol). To the 1-alpha,3-beta-Di(tert-Butyl-dimethyl-silanyloxy)-25-hydroxy-16-ene-20-cyclopropyl-23,24-yne-26,27-hexafluoro-cholecalciferol (356 mg, 0.47 mmol) tetrabutylammonium fluoride (5 mL, 5 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (270 mg) after evaporation of the solvent was purified by FC (20 g, 50% AcOEt in hexane and AcOEt) to give the titled compound (26) (216 mg, 0.41 mmol, 87%). [α]³⁰_{D}=+40.0 c 0.53, EtOH. UV λmax (EtOH): 262 nm (ε 12919); ¹H NMR (CDCl₃): 6.38 (1H, d, J=11.5 Hz), 6.10 (1H, d, J=11.1 Hz), 5.49 (1H, m), 5.35 (1H, s), 5.02 (1H, s), 4.45 (1H, m), 4.25 (1H, m), 3.57 (1H, s), 2.83-1.45 (18H, m), 0.82 (3H, s), 0.80-0.51 (4H, m); MS HRES Calculated for C₂₈H₃₂O₃F₆ M+H 531.2329. Observed M+H 531.2337.

### Synthetic Example 31

### Synthesis of (3aR,4S,7aR)-7a-Methyl-1-[1-(5,5,5-trifluoro-4-hydroxy-4-trifluoromethyl-pent-2E-enyl)-cyclopropyl]-3a,4,5,6,7,7a-hexahydro-3H-inden-4-ol

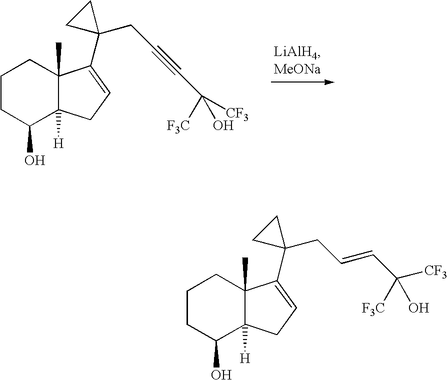

To a lithium aluminum hydride (4.5 mL, 4.5 mmol, 1.0M in THF) at 5° C. was added first solid sodium methoxide (245 mg, 4.6 mmol) and then dropwise solution of (3aR,4S,7aR)-7a-Methyl-1-[1-(5,5,5-trifluoro-4-hydroxy-4-trifluoromethyl-pent-2-ynyl)-cyclopropyl]-3a,4,5,6,7,7a-hexahydro-3H-inden-4-ol (360 mg, 0.91 mmol) in tetrahydrofurane (5 mL). After addition was completed the mixture was stirred under reflux for 2.5 h. Then it was cooled in the ice-bath and quenched with water (2.0 mL) and sodium hydroxide (2.0 mL, 2.0 M water solution); diluted with ether (50 mL) stirred for 30 min, MgSO₄ (5 g) was than added and stirring was continued for 30 min. The residue after evaporation of the filtrates (0.42 g) was purified by FC (20 g, 20% AcOEt in hexane) to give the titled compound (315 mg, 0.79 mmol, 87%). [α]²⁸_{D}=+2.0 c 0.41, CHCl₃. ¹H NMR (CDCl₃): 6.24 (1H, dt, J=15.7, 6.7 Hz), 5.60 (1H, d, J=15.7 Hz), 5.38 (1H, br. s), 4.13 (1H, br. s), 3.27 (1H, s), 2.32-1.34 (12H, m), 1.15 (3H, s), 0.80-0.45 (4H, m); ¹³C NMR (CDCl₃): 155.89 (0), 138.10 (1), 126.21 (1), 122.50 (0, q, J=287 Hz), 119.15 (1), 76.09 (0, sep. J=31 Hz), 69.57 (1), 55.33 (1), 47.30 (0), 40.31 (2), 36.05 (2), 33.71 (2), 30.10 (2), 20.36 (0), 19.46 (3), 17.94 (2), 11.96 (2), 11.46 (2); MS REI Calculated for C₁₉H₂₄O₂F₆ M+ 398.1680. Observed M+ 398.1675.

### Synthetic Example 32

### Synthesis of (3aR,7aR)-7a-Methyl-1-[1-(5,5,5-trifluoro-4-trifluoromethyl-4-trimethylsilanyloxy-pen-2E-enyl)-cyclopropyl]-3a,4,5,6,7,7a-hexahydro-3H-inden-4-one

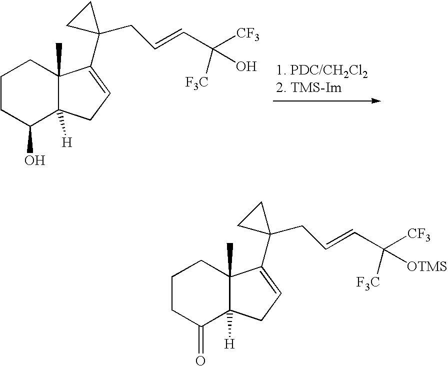

To a stirred suspension of (3aR,4S,7aR)-7a-Methyl-[1-(5,5,5-trifluoro-4-hydroxy-4-trifluoromethyl-pent-2E-enyl)-cyclopropyl]-3a,4,5,6,7,7a-hexahydro-3H-inden-4-ol (600 mg, 1.51 mmol) and Celite (2.0 g) in dichloromethane (10 mL) at room temperature wad added pyridinium dichromate (1.13 g, 3.0 mmol). The resulting mixture was stirred for 3.5 h filtered through silica gel (10 g), and then silica gel pad was washed with 25% AcOEt in hexane. The combined filtrate and washes were evaporated, to give a crude (3aR,7aR)-7a-Methyl-1-[1-(5,5,5-trifluoro-4-hydroxy-4-trifluoromethyl-pent-2E-enyl)-cyclopropyl]-3a,4,5,6,7,7a-hexahydro-3H-inden-4-one (550 mg, 1.39 mmol, 92%). To a stirred solution of (3aR,7aR)-7a-Methyl-1-[1-(5,5,5-trifluoro-4-hydroxy-4-trifluoromethyl-pent-2E-enyl)-cyclopropyl]-3a,4,5,6,7,7a-hexahydro-3H-inden-4-one (550 mg, 1.39 mmol) in dichloromethane (15 mL) at room temperature was added trimethylsilyl-imidazole (1.76 mL, 12.0 mmol). The resulting mixture was stirred for 1.0 h filtered through silica gel (10 g) and the silica gel pad was washed with 10% AcOEt in hexane. Combined filtered and washes were evaporated to give the titled compound (623 mg, 1.33 mmol, 88%). [α]²⁸_{D}=−1.6 c 0.51, CHCl_{3.1}H NMR (CDCl₃): 6.14 (1H, dt, J=15.5, 6.7 Hz), 5.55 (1H, d, J=15.5 Hz), 5.35 (1H, m), 2.80 (1H, dd, J=10.7, 6.4 Hz), 2.47-1.74 (10H, m), 0.90 (3H, s), 0.76-0.40 (4H, m), 0.2 (9H, s); ¹³C NMR (CDCl₃): 210.99 (0), 154.28 (0), 137.41 (1), 126.26 (1), 122.59 (0, q, J=289 Hz), 120.89 (1), 64.31 (1), 53.96 (0), 40.60 (2), 40.13 (2), 35.00 (2), 27.03 (2), 24.21 (2), 20.57 (0), 18.53 (3), 12.41 (2), 10.79 (2), 1.65 (3); MS HRES Calculated for C₂₂H₃₀O₂F₆Si M+H 469.1992. Observed M+H 469.1995.

### Synthetic Example 33

### Synthesis of 1-alpha,25-Dihydroxy-16-ene-20-cyclopropyl-23,24-E-ene-26,27-hexafluoro-19-nor-cholecalciferol (27)

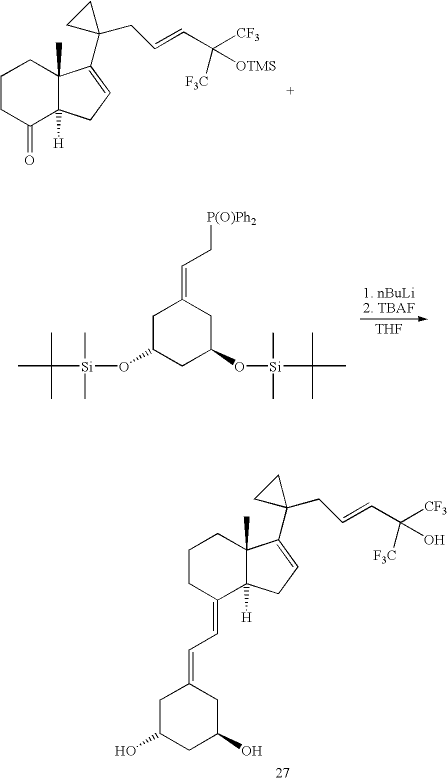

To a stirred solution of a (1R,3R)-1,3-bis-((tert-butyldimethyl)silanyloxy)-5-[2-(diphenylphosphinoyl)ethylidene]-cyclohexane (514 mg, 0.90 mmol) in tetrahydrofurane (6 mL) at −78° C. was added n-BuLi (0.57 mL, 0.91 mmol). The resulting mixture was stirred for 15 min and solution of (3aR,7aR)-7a-Methyl-1-[1-(5,5,5-trifluoro-4-trifluoromethyl-4-trimethylsilanyloxy-pent-2E-enyl)-cyclopropyl]-3a,4,5,6,7,7a-hexahydro-3H-inden-4-one (200 mg, 0.43 mmol, in tetrahydrofurane (2 mL) was added dropwise. The reaction mixture was stirred at −72° C. for 3.5 h diluted with hexane (35 mL) washed brine (30 mL) and dried over Na₂SO₄. The residue (750 mg) after evaporation of the solvent was purified by FC (15 g, 5% AcOEt in hexane) to give a mixture of 1-alpha,3-beta-Di(tert-Butyl-dimethyl-silanyloxy)-25-trimethylsilanyloxy-16-ene-20-cyclopropyl-23,24-E-ene-26,27-hexafluoro-19-nor-cholecalciferol and 1-alpha,3-beta-Di(tert-Butyl-dimethyl-silanyloxy)-25-hydroxy-16-ene-20-cyclopropyl-23,24-E-ene-26,27-hexafluoro-19-nor-cholecalciferol (250 mg). To the mixture of 1-alpha,3-beta-Di(tert-Butyl-dimethyl-silanyloxy)-25-trimethylsilanyloxy-16-ene-20-cyclopropyl-23,24-E-ene-26,27-hexafluoro-19-nor-cholecalciferol and 1-alpha,3-beta-Di(tert-Butyl-dimethyl-silanyloxy)-25-hydroxy-16-ene-20-cyclopropyl-23,24-E-ene-26,27-hexafluoro-19-nor-cholecalciferol (250 mg) tetrabutylammonium fluoride (4 mL, 4 mmol, 1M solution in THF) was added, at room temperature. The mixture was stirred for 24 h. diluted with AcOEt (25 mL) and washed with water (5×20 mL), brine (20 mL) and dried over Na₂SO₄. The residue (270 mg) after evaporation of the solvent was purified by FC (10 g, 50% AcOEt in hexane and AcOEt) to give the titled compound (27) (157 mg, 0.30 mmol, 70%). [α]³⁰_{D}=+63.3 c 0.45, EtOH. UV λmax (EtOH): 243 nm (ε30821), 251 εnm (36064), 260 nm (ε 24678); ¹H NMR (CDCl₃): 6.29 (1H, d, J=11.3 Hz), 6.24 (1H, dt, J=15.9, 6.4 Hz), 5.92 (1H, d, J=111.1 Hz), 5.61 (1H, d, J=15.7 Hz), 5.38 (1H, m), 4.13 (1H, m), 4.05 (1H, m), 2.88 (1H, s), 2.82-1.34 (19H, m), 0.770 (3H, s), 0.80-0.36 (4H, m); MS HRES Calculated for C₂₇H₃₄O₃F₆ M+H 521.2485. Observed M+H 521.2489.

### Synthetic Example 34

### Synthesis of 1-alpha,25-Dihydroxy-16-ene-20-cyclopropyl-23,24-E-ene-26,27-hexafluoro-cholecalciferol (28)

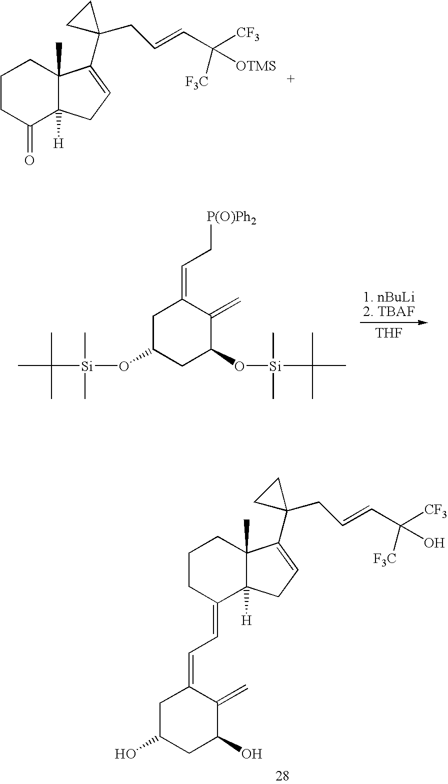

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (525 mg, 0.90 mmol) in tetrahydrofurane (6 mL) at −78° C. was added n-BuLi (0.57 mL, 0.91 mmol). The resulting mixture was stirred for 15 min and solution of (3aR,7aR)-7a-Methyl-1-[1-(5,5,5-trifluoro-4-trifluoromethyl-4-tridimethylsilanyloxy-pent-2E-enyl)-cyclopropyl]-3a,4,5,6,7,7a-hexahydro-3H-inden-4-one (200 mg, 0.43 mmol, in tetrahydrofurane (2 mL) was added dropwise. The reaction mixture was stirred at −72° C. for 2.5 h diluted with hexane (35 mL) washed brine (30 mL) and dried over Na₂SO₄. The residue (760 mg) after evaporation of the solvent was purified by FC (15 g, 10% AcOEt in hexane) to give a mixture of 1-alpha,3-beta-Di(tert-Butyl-dimethyl-silanyloxy)-25-trimethylsilanyloxy-16-ene-20-cyclopropyl-23,24-E-ene-26,27-hexafluoro-cholecalciferol and 1-alpha,3-beta-Di(tert-Butyl-dimethyl-silanyloxy)-25-hydroxy-16-ene-20-cyclopropyl-23,24-E-ene-26,27-hexafluoro-cholecalciferol (274 mg). To the mixture of 1-alpha,3-beta-Di(tert-Butyl-dimethyl-silanyloxy)-25-trimethylsilanyloxy-16-ene-20-cyclopropyl-23,24-E-ene-26,27-hexafluoro-cholecalciferol and 1-alpha,3-beta-Di(tert-Butyl-dimethyl-silanyloxy)-25-hydroxy-16-ene-20-cyclopropyl-23,24-E-ene-26,27-hexafluoro-cholecalciferol (274 mg) tetrabutylammonium fluoride (4 mL, 4 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (280 mg) after evaporation of the solvent was purified by FC (15 g, 50% AcOEt in hexane and AcOEt) to give the titled compound (28) (167 mg, 0.31 mmol, 73%). [α]³⁰_{D}=+18.3 c 0.41, EtOH. UV λmax (EtOH): 207 nm (ε 17778), 264 nm (ε 15767); ¹H NMR (CDCl₃): 6.36 (1H, d, J=11.1 Hz), 6.24 (1H, dt, J=15.7, 6.7 Hz), 6.07 (1H, d, J=11.3 Hz), 5.60 (1H, d, J=15.5 Hz), 5.35 (1H, m), 5.33 (1H, s), 5.00 (1H, s), 4.44 (1H, m), 4.23 (1H, m), 3.14 (1H, s), 2.80 (1H, m), 2.60 (1H, m), 2.40-1.40 (15H, m), 0.77 (3H, s), 0.80-0.36 (4H, m); MS HRES Calculated for C₂₈H₃₄O₃F₆ M+H 533.2485. Observed M+H 533.2483.

### Synthetic Example 35

### Synthesis of (3aR,4S,7aR)-7a-Methyl-1-[1-(5,5,5-trifluoro-4-hydroxy-4-trifluoromethyl-pent-2Z-enyl)-cyclopropyl]-3a,4,5,6,7,7a-hexahydro-3H-inden-4-ol

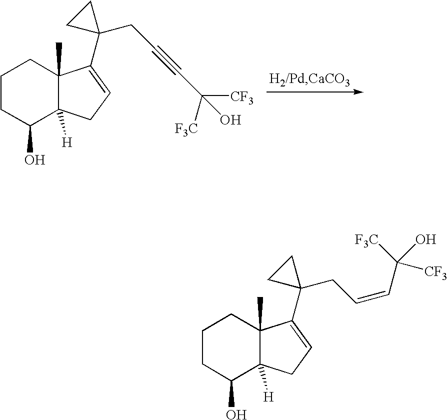

The mixture of (3aR,4S,7aR)-7a-Methyl-1-[1-(5,5,5-trifluoro-4-hydroxy-4-trifluoromethyl-pent-2-ynyl)-cyclopropyl]-3a,4,5,6,7,7a-hexahydro-3H-inden-4-ol (300 mg, 0.76 mmol), ethyl acetate (5 mL), hexane (12 mL), absolute ethanol (0.5 mL) quinoline (30 uL) and Lindlar catalyst (75 mg, 5% Pd on CaCO₃) was hydrogenated at room temperature for 2 h. The reaction mixture was filtered through a celite pad and the pad was washed with AcOEt. The solvent was evaporated to give the titled compound (257 mg, 0.65 mmol, 87%). [α]²⁸_{D}=+1.8 c 0.61, CHCl₃. ¹H NMR (CDCl₃): 6.08 (1H, dt, J=12.3, 6.7 Hz), 5.47 (1H, m,), 5.39 (1H, d, J=12.1 Hz), 4.15 (1H, br. s), 3.28 (1H, s), 2.52-1.34 (12H, m), 1.16 (3H, s), 0.78-0.36 (4H, m); ¹³C NMR (CDCl₃): 156.66 (0), 141.77 (1), 126.51 (1), 122.79 (0, q, J=285 Hz), 115.77 (1), 69.59 (1), 55.41 (1), 47.28 (0), 36.44 (2), 35.90 (2), 33.75 (2), 30.22 (2), 20.89 (0), 19.41 (3), 17.94 (2), 12.05 (2), 11.11 (2); MS HRES Calculated for C₁₉H₂₄O₂F₆ M+H 399.1753. Observed M+H 399.1757.

### Synthetic Example 36

### Synthesis of (3aR,7aR)-7a-Methyl-1-[1-(5,5,5-trifluoro-4-trifluoromethyl-4-trimethylsilanyloxy-pen-2Z-enyl)-cyclopropyl]-3a,4,5,6,7,7a-hexahydro-3H-inden-4-one

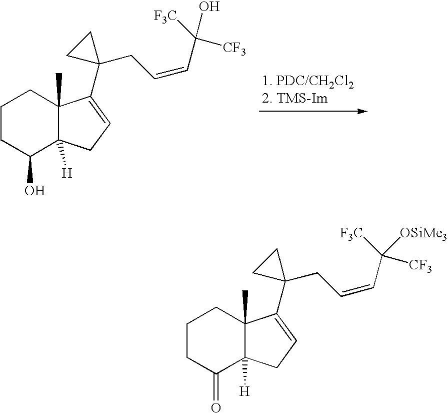

To a stirred suspension of (3aR,4S,7aR)-7a-Methyl-1-[1-(5,5,5-trifluoro-4-hydroxy-4-trifluoromethyl-pent-2Z-enyl)-cyclopropyl]-3a,4,5,6,7,7a-hexahydro-3H-inden-4-ol (617 mg, 1.55 mmol) and Celite (2.0 g) in dichloromethane (10 mL) at room temperature wad added pyridinium dichromate (1.17 g, 3.1 mmol). The resulting mixture was stirred for 2.5 h filtered through silica gel (5 g), and then silica gel pad was washed with 20% AcOEt in hexane. The combined filtrate and washes were evaporated, to give a crude (3aR,7aR)-7a-Methyl-1-[1-(5,5,5-trifluoro-4-hydroxy-4-trifluoromethyl-pentenyl)-cyclopropyl]-3a,4,5,6,7,7a-hexahydro-3H-inden-4-one (600 mg, 1.51 mmol, 98%). To a stirred solution of (3aR,7aR)-7a-Methyl-1-[1-(5,5,5-trifluoro-4-hydroxy-4-trifluoromethyl-pent-2Z-enyl)-cyclopropyl]-3a,4,5,6,7,7a-hexahydro-3H-inden-4-one (600 mg, 1.51 mmol) in dichloromethane (15 mL) at room temperature was added trimethylsilyl-imidazole (1.76 mL, 12.0 mmol). The resulting mixture was stirred for 1.0 h filtered through silica gel (10 g) and the silica gel pad was washed with 10% AcOEt in hexane. Combined filtered and washes were evaporated to give the titled compound (640 mg, 1.37 mmol, 88%). [α]²⁸_{D}=−0.2 c 0.55, CHCl₃. ¹H NMR (CDCl₃): 5.97 (1H, dt, J=12.2, 6.2 Hz), 5.40 (1H, m), 5.38 (1H, d, J=12.2 Hz), 2.82 (1H, dd, J=10.7, 6.6 Hz), 2.60-1.74 (10H, m), 0.89 (3H, s), 0.75-0.36 (4H, m), 0.21 (9H, s); ¹³C NMR (CDCl₃): 210.56 (0), 154.30 (0), 139.28 (1), 125.81 (1), 122.52 (0, q, J=289 Hz), 118.17 (1), 64.11 (1), 53.69 (0), 40.43 (2), 35.51 (2), 34.85 (2), 26.94 (2), 24.07 (2), 20.89 (0), 18.39 (3), 12.26 (2), 10.61 (2), 1.43 (3); MS HRES Calculated for C₂₂H₃₀O₂F₆Si M+H 469.1992. Observed M+H 469.1992.

### Synthetic Example 37

### Synthesis of 1-alpha,25-Dihydroxy-16-ene-20-cyclopropyl-23,24-Z-ene-26,27-hexafluoro-19-nor-cholecalciferol (29)

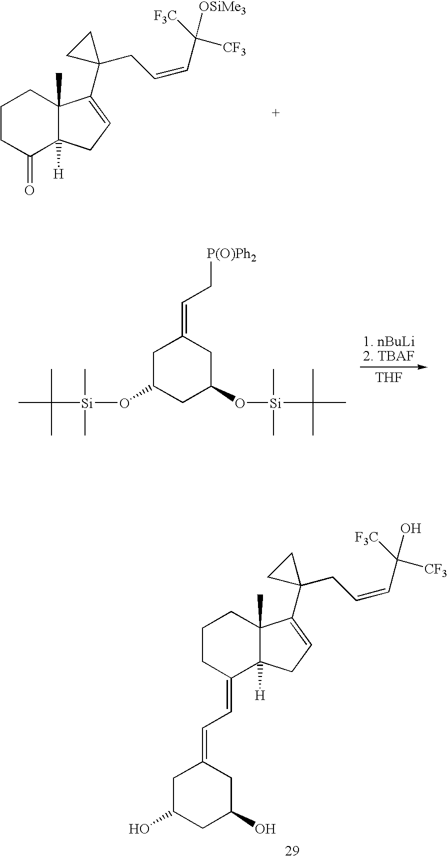

To a stirred solution of a (1R,3R)-1,3-bis-((tert-butyldimethyl)silanyloxy)-5-[2-(diphenylphosphinoyl)ethylidene]-cyclohexane (514 mg, 0.90 mmol) in tetrahydrofurane (6 mL) at −78° C. was added n-BuLi (0.57 mL, 0.91 mmol). The resulting mixture was stirred for 15 min and solution of (3aR,7aR)-7a-Methyl-1-[1-(5,5,5-trifluoro-4-trifluoromethyl-4-trimethylsilanyloxy-pent-2Z-enyl)-cyclopropyl]-3a,4,5,6,7,7a-hexahydro-3H-inden-4-one (194 mg, 0.41 mmol, in tetrahydrofurane (2 mL) was added dropwise. The reaction mixture was stirred at −72° C. for 3.0 h diluted with hexane (35 mL) washed brine (30 mL) and dried over Na₂SO₄. The residue (750 mg) after evaporation of the solvent was purified by FC (15 g, 10% AcOEt in hexane) to give a mixture of 1-alpha,3-beta-Di(tert-Butyl-dimethyl-silanyloxy)-25-trimethylsilanyloxy-16-ene-20-cyclopropyl-23,24-Z-ene-26,27-hexafluoro-19-nor-cholecalciferol and 1-alpha,3-beta-Di(tert-Butyl-dimethyl-silanyloxy)-25-hydroxy-16-ene-20-cyclopropyl-23,24-Z-ene-26,27-hexafluoro-19-nor-cholecalciferol (230 mg). To the mixture of 1-alpha,3-beta-Di(tert-Butyl-dimethyl-silanyloxy)-25-trimethylsilanyloxy-16-ene-20-cyclopropyl-23,24-Z-ene-26,27-hexafluoro-19-nor-cholecalciferol and 1-alpha,3-beta-Di(tert-Butyl-dimethyl-silanyloxy)-25-hydroxy-16-ene-20-cyclopropyl-23,24-Z-ene-26,27-hexafluoro-19-nor-cholecalciferol (230 mg) tetrabutylammonium fluoride (4 mL, 4 mmol, 1M solution in THF) was added, at room temperature. The mixture was stirred for 40 h. diluted with AcOEt (25 mL) and washed with water (5×20 mL), brine (20 mL) and dried over Na₂SO₄. The residue (260 mg) after evaporation of the solvent was purified by FC (10 g, 50% AcOEt in hexane and AcOEt) to give the titled compound (29) (1327 mg, 0.25 mmol, 62%). [α]²⁸_{D}=+53.6 c 0.33, EtOH. UV λmax (EtOH): 243 nm (ε 2698) 2 . . . 251 εnm (32081), 260 nm (ε 21689); ¹H NMR (CDCl₃): 6.29 (1H, d, J=10.7 Hz), 6.08 (1H, dt, J=12.5, 6.7 Hz), 5.93 (1H, d, J=11.1 Hz), 5.46 (1H, m,), 5.40 (1H, d, J=12.7 Hz)), 4.12 (1H, m), 4.05 (1H, m), 3.14 (1H, s), 2.80-1.40 (19H, m), 0.77 (3H, s), 0.80-0.36 (4H, m); MS HRES Calculated for C₂₇H₃₄O₃F₆ M+H 521.2485. Observed M+H 521.2487.

### Synthetic Example 38

### Synthesis of 1-alpha,25-Dihydroxy-16-ene-20-cyclopropyl-23,24-Z-ene-26,27-hexafluoro-cholecalciferol (30)

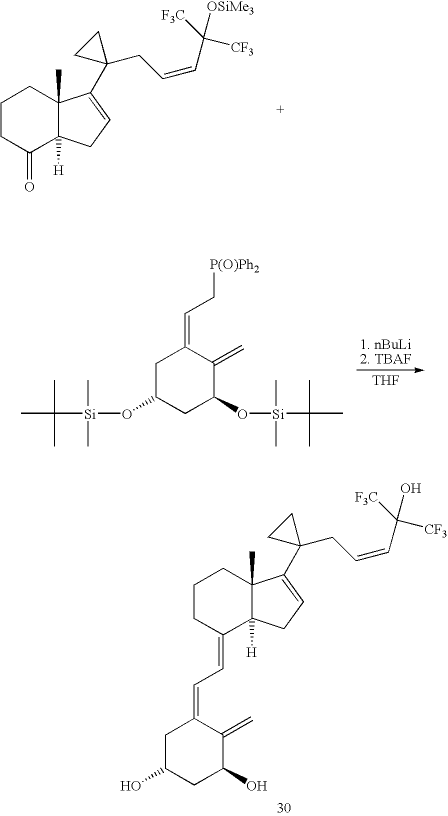

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (525 mg, 0.90 mmol) in tetrahydrofurane (6 mL) at −78° C. was added n-BuLi (0.57 mL, 0.91 mmol). The resulting mixture was stirred for 15 min and solution of (3aR,7aR)-7a-Methyl-1-[1-(5,5,5-trifluoro-4-trifluoromethyl-4-trimethylsilanyloxy-pent-2Z-enyl)-cyclopropyl]-3a,4,5,6,7,7a-hexahydro-3H-inden-4-one (200 mg, 0.43 mmol, in tetrahydrofurane (2 mL) was added dropwise. The reaction mixture was stirred at −72° C. for 2.5 h diluted with hexane (35 mL) washed brine (30 mL) and dried over Na₂SO₄. The residue (680 mg) after evaporation of the solvent was purified by FC (15 g, 10% AcOEt in hexane) to give a mixture of 1-alpha,3-beta-Di(tert-Butyl-dimethyl-silanyloxy)-25-trimethylsilanyloxy-16-ene-20-cyclopropyl-23,24-Z-ene-26,27-hexafluoro-cholecalciferol and 1-alpha,3-beta-Di(tert-Butyl-dimethyl-silanyloxy)-25-hydroxy-16-ene-20-cyclopropyl-23,24-Z-ene-26,27-hexafluoro-cholecalciferol (310 mg). To the mixture of 1-alpha,3-beta-Di(tert-Butyl-dimethyl-silanyloxy)-25-trimethylsilanyloxy-16-ene-20-cyclopropyl-23,24-Z-ene-26,27-hexafluoro-cholecalciferol and 1-alpha,3-beta-Di(tert-Butyl-dimethyl-silanyloxy)-25-hydroxy-16-ene-20-cyclopropyl-23,24-Z-ene-26,27-hexafluoro-cholecalciferol (310 mg) tetrabutylammonium fluoride (4 mL, 4 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (370 mg) after evaporation of the solvent was purified by FC (10 g, 50% AcOEt in hexane and AcOEt) to give the titled compound (30) (195 mg, 0.37 mmol, 85%). [α]³⁰_{D}=+9.4 c 0.49, EtOH. UV λmax (EtOH): 262 nm (ε 11846); ¹H NMR (CDCl₃): 6.36 (1H, d, J=111.1 Hz), 6.08 (2H, m), 5.44 (1H, m), 5.40 (1H, d, J=12.3 Hz), 5.32 (1H, s), 5.00 (1H, s), 4.43 (1H, m), 4.23 (1H, m), 3.08 (1H, s), 2.80 (1H, m), 2.60 (1H, m), 2.55-1.40 (15H, m), 0.77 (3H, s), 0.80-0.34 (4H, m); MS HRES Calculated for C₂₈H₃₄O₃F₆ M+H 533.2485. Observed M+H 533.2502.

### Synthetic Example 39

### Synthesis of 1-alpha,25-Dihydroxy-16-ene-20-cyclopropyl-19-nor-cholecalciferol (31)

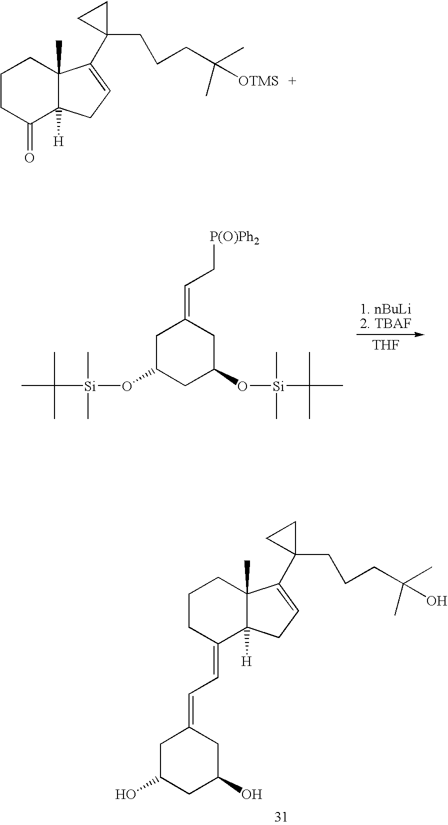

To a stirred solution of a (1R,3R)-1,3-bis-((tert-butyldimethyl)silanyloxy)-5-[2-(diphenylphosphinoyl)ethylidene]-cyclohexane (697 mg, 1.22 mmol) in tetrahydrofurane (9 mL) at −78° C. was added n-BuLi (0.77 mL, 1.23 mmol). The resulting mixture was stirred for 15 min and solution of (3aR,7aR)-7a-Methyl-1-[1-(4-methyl-4-trimethylsilanyloxy-pentyl)-cyclopropyl]-3a,4,5,6,7,7a-hexahydro-3H-inden-4-one (220 mg, 0.61 mmol, in tetrahydrofurane (2 mL) was added dropwise. The reaction mixture was stirred at −72° C. for 3.5 h diluted with hexane (35 mL) washed brine (30 mL) and dried over Na₂SO₄. The residue (900 mg) after evaporation of the solvent was purified by FC (15 g, 10% AcOEt in hexane) to give 1-alpha,3-beta-Di(tert-Butyl-dimethyl-silanyloxy)-25-trimethylsilanyloxy-16-ene-20-cyclopropyl-19-nor-cholecalciferol (421 mg, 0.59 mmol). To the 1-alpha,3-beta-Di(tert-Butyl-dimethyl-silanyloxy)-25-trimethylsilanyloxy-16-ene-20-cyclopropyl-26,27-hexadeutero-19-nor-cholecalciferol (421 mg, 0.59 mmol) tetrabutylammonium fluoride (4 mL, 4 mmol, 1M solution in THF) was added, at room temperature. The mixture was stirred for 40 h. diluted with AcOEt (25 mL) and washed with water (5×20 mL), brine (20 mL) and dried over Na₂SO₄. The residue (450 mg) after evaporation of the solvent was purified by FC (15 g, 50% AcOEt in hexane and AcOEt) to give the titled compound (31) (225 mg, 0.54 mmol, 89%). [α]²⁹_{D}=+69.5 c 0.37, EtOH. UV λmax (EtOH): 243 nm (ε 2794) 6 . . 251 εnm (33039), 261 nm (ε 22701); ¹H NMR (CDCl₃): 6.30 (1H, d, J=11.3 Hz), 5.93 (1H, d, J=11.3 Hz), 5.36 (1H, m), 4.12 (1H, m), 4.04 (1H, m), 2.75 (2H, m), 2.52-1.04 (22H, m), 1.18 (6H, s), 0.79 (3H, s), 0.65-0.26 (4H, m); ¹³C NMR (CDCl₃): 157.16 (0), 142.33 (0), 131.25 (0), 124.73 (1), 123.76 (1), 115.50 (1), 71.10 (0), 67.39 (1), 67.19 (1), 59.47 (1), 50.12 (0), 44.60 (2), 43.84 (2), 42.15 (2), 38.12 (2), 37.18 (2), 35.57 (2), 29.26 (3), 29.11 (2), 29.08 (3), 28.48 (2), 23.46 (2), 22.26 (2), 21.27 (0), 17.94 (3), 12.70 (2), 10.27 (2); MS HRES Calculated for C₂₇H₄₂O₃ M+H 415.3207. Observed M+H 415.3207.

### Synthetic Example 40

### Synthesis of 1-alpha,25-Dihydroxy-16-ene-20-cyclopropyl-cholecalciferol (32)

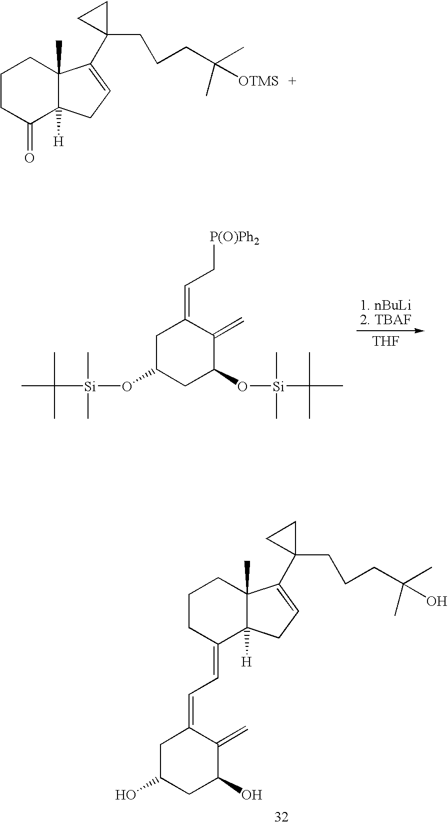

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (675 mg, 1.16 mmol) in tetrahydrofurane (8 mL) at −78° C. was added n-BuLi (0.73 mL, 1.17 mmol). The resulting mixture was stirred for 15 min and solution of (3aR,7aR)-7a-Methyl-1-[1-(4-methyl-4-trimethylsilanyloxy-pentyl)-cyclopropyl]-3a,4,5,6,7,7a-hexahydro-3H-inden-4-one (210 mg, 0.58 mmol, in tetrahydrofurane (2 mL) was added dropwise. The reaction mixture was stirred at −72° C. for 3.5 h diluted with hexane (35 mL) washed brine (30 mL) and dried over Na₂SO₄. The residue (850 mg) after evaporation of the solvent was purified by FC (15 g, 10% AcOEt in hexane) to give 1-alpha,3-beta-Di(tert-Butyl-dimethyl-silanyloxy)-25-trimethylsilanyloxy-16-ene-20-cyclopropyl-cholecalciferol (382 mg, 0.53 mmol). To the 1-alpha,3-beta-Di(tert-Butyl-dimethyl-silanyloxy)-25-trimethylsilanyloxy-16-ene-20-cyclopropyl-cholecalciferol (382 mg, 0.53 mmol) tetrabutylammonium fluoride (4 mL, 4 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (380 mg) after evaporation of the solvent was purified by FC (15 g, 50% AcOEt in hexane and AcOEt) to give the titled compound (32) (204 mg, 0.48 mmol, 83%). [α]²⁹_{D}=+16.1 c 0.36, EtOH. UV λmax (EtOH): 208 nm (ε 17024), 264 nm (ε 16028); ¹H NMR (CDCl₃): 6.37 (1H, d, J=11.3 Hz), 6.09 (1H, d, J=11.1 Hz), 5.33 (2H, m), 5.01 (1H, s), 4.44 (1H, m), 4.23 (1H, m), 2.80 (1H, m), 2.60 (1H, m), 2.38-1.08 (20H, m), 1.19 (6H, s), 0.79 (3H, s), 0.66-0.24 (4H, m); ¹³C NMR (CDCl₃): 157.07 (0), 147.62 (0), 142.49 (0), 133.00 (0), 124.90 (1), 124.73 (1), 117.19 (1), 111.64 (2), 71.10 (1), 70.70 (0), 66.88 (1), 59.53 (1), 50.28 (0), 45.19 (2), 43.85 (2), 42.86 (2), 38.13 (2), 35.59 (2), 29.27 (2), 29.14 (3), 28.65 (2), 23.57 (2), 22.62 (2), 21.29 (0), 17.84 (3), 12.74 (2), 10.30 (2); MS HRES Calculated for C₂₈H₄₂O₃ M+Na 449.3026. Observed M+Na 449.3023.

### Synthetic Example 41

### Synthesis of 1,25-Dihydroxy-21-(2R,3-dihydroxy-3-methyl-butyl)-20R-Cholecalciferol (33)

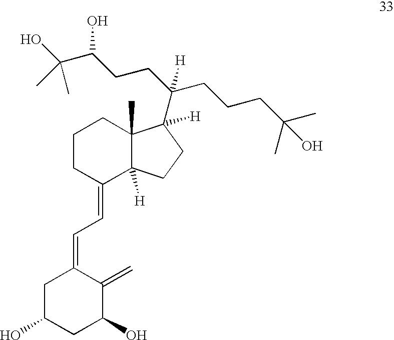

### [1R,3aR,4S,7aR]-2(R)-[4-(1,1-dimethylethyl)dimethyl-silanyloxy)-7a-methyl-octahydro-inden-1-yl]-6-methyl-heptane-1,6-diol (34) and [1R,3aR,4S,7aR]-2(S)-[4-(1,1-dimethylethyl)dimethyl-silanyloxy)-7a-methyl-octahydro-inden-1-yl]-6-methyl-heptane-1,6-diol (35)

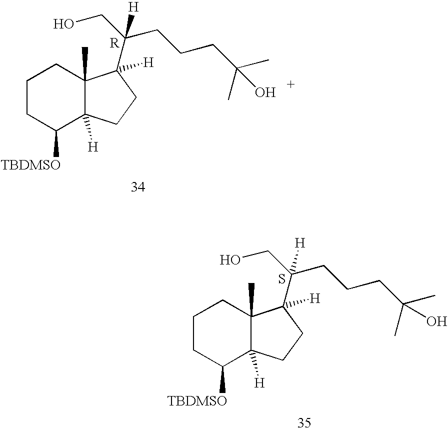

A solution of the alkenol in tetrahydrofuran (9 mL) was cooled in an ice bath and a 1 M solution of borane-THF in tetrahydrofuran (17 mL) was added dropwise in an originally effervescent reaction. The solution was stirred overnight at room temperature, re-cooled in an ice bath water (17 mL) was added dropwise followed by sodium percarbonate (7.10 g, 68 mmol). The mixture was immersed into a 50° C. bath and stirred for 70 min to generate a solution. The two-phase system was allowed to cool then equilibrated with 1:1 ethyl acetate-hexane (170 mL). The organic layer was washed with water (2×25 mL) then brine (20 mL), dried and evaporated to leave a colorless oil (2.76 g). This material was passed through a short flash column using 1:1 ethyl acetate-hexane and silica gel G. The effluent, obtained after exhaustive elution, was evaporated, taken up in ethyl acetate, filtered and chromatographed on the 2×18″ 15-20 g silica YMC HPLC column using 2:1 ethyl acetate-hexane as mobile phase and running at 100 mL/min. Isomer 34 emerged at an effluent maximum of 2.9 L, colorless oil, 1.3114 g, [α]_{D}+45.2° (methanol, c 0.58; ¹H NMR δ −0.002 (3H, s), 0.011 (3H, s), 0.89 (9H, s), 0.93 (3H, s), 1.17 (1H, m), 1.22 (6H, s), 1.25-1.6 (16H, m), 1.68 (1H, m), 1.80 (2H, m), 1.89 (1H, m), 3.66 (1H, dd, J=4.8 and 11 Hz), 3.72 (1H, dd, J=3.3 and 11 Hz), 4.00 (1H, m); LR-ES(−) m/z 412 (M), 411 (M−H); HR-ES(+): calcd for (M+Na) 435.3265. found: 435.3269.

Isomer 35 at was eluted at an effluent maximum of 4.9 L, colorless oil, 0.8562 g that crystallized upon prolonged standing: mp 102-3°, [α]_{D}+25.2° (methanol, c 0.49); ¹H NMR δ −0.005 (3H, s), 0.009 (3H, s), 0.89 (9H, s), 0.93 (3H, s), 1.16 (1H, m), 1.22 (6H, s), 1.3-1.5, (14H, m), 1.57 (2H, m), 1.67 (1H, m), 1.80 (2H, m), 1.91 (1H, m), 3.54 (1H, dd, J=4.8 and 11 Hz), 3.72 (1H, dd, J=2.9 and 11 Hz), 4.00 (1H, m);); LR-ES(−) m/z 412 (M), 411 (M−H). Anal. Calcd for C₂₄H₄₈O₃Si: C, 69.84; H, 11.72. found: C, 69.91; H, 11.76.

### [1R,3aR,4S,7aR]-6(R)-[4-(tert-Butyl-dimethyl-silanyloxy)-7a-methyl-octahydro-inden-1-yl]-7-iodo-2-methyl-heptan-2-ol (36)

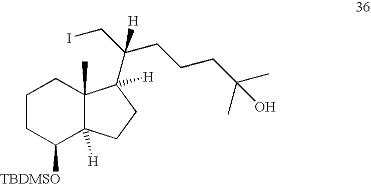

A stirred mixture of triphenylphosphine (0.333 g, 1.27 mmol) and imidazole (0.255 g, 3 mmol) in dichloromethane (3 mL) was cooled in an ice bath and iodine (0.305 g, 1.20 mmol) was added. This mixture was stirred for 10 min then a solution of 34 (0.4537 g, 1.10 mmol) in dichloromethane (3 mL) was added dropwise over a 10 min period. The mixture was stirred in the ice bath for 30 min then at ambient temperature for 2% h. TLC (1:1 ethyl acetate-hexane) confirmed absence of educt. A solution of sodium thiosulfate (0.1 g) in water (5 mL) was added, the mixture equilibrated and the organic phase washed with 0.1 N sulfuric acid (10 mL) containing a few drops of brine then with 1:1 water-brine (2×10 mL), once with brine (10 mL) then dried and evaporated. The residue was purified by flash chromatography using 1:9 ethyl acetate-hexane as mobile phase to furnish 36 as a colorless syrup, 0.5637 g, 98%: ¹H NMR δ-0.005 (3H, s), 0.010 (3H, s), 0.89 (9H, s), 0.92 (3H, s), 1.23 (6H, s), 1.1-1.6 (16H, m), 1.68 (1H, m), 1.79 (2H, m), 1.84 (1H, m), 3.37 (1H, dd, J=4 and 10 Hz), 3.47 (1H, dd, J=3 and 10 Hz), 4.00 (1H, m); LR-EI(+) m/z 522 (M), 465 (M-C₄H₉), 477 (M-C₄H₉—H₂O); HR-EI(+): calcd for C₂₄H₄₇IO₂Si: 522.2390. found: 522.2394.

### [1R,3aR,4S,7aR]-6(S)-[4-(tert-Butyl-dimethyl-silanyloxy)-7a-methyl-octahydro-inden-1-yl]-2-methyl-non-8-yn-2-ol (37)

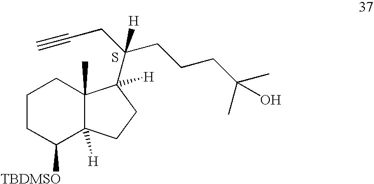

Lithium acetylide DMA complex (0.110 g, 1.19 mmol) was added to a solution of 36 (0.2018 g (0.386 mmol) in dimethyl sulfoxide (1.5 mL) and tetrahydrofuran (0.15 mL). The mixture was stirred overnight. TLC (1:4 ethyl acetate-hexane) showed a mixture of two spots traveling very close together (Rf 0.52 and 0.46). Fractions at the beginning of the eluted band contained pure alkenol, which is the elimination product of 36, and was produced as the major product. Fractions at the end of the elution band, however, were also homogeneous and gave the desired acetylene 37 upon evaporation. The NMR spectra of 37 and its 6-epimer which served for identification were previously reported.

### [1R,3aR,4S,7aR]-7-Benzenesulfonyl-6(S)-[4-(tert-butyl-dimethyl-silanyloxy)-7a-methyl-octahydro-inden-1-yl]-2-methyl-heptan-2-ol (38)

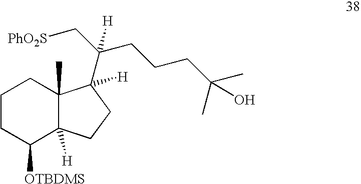

A mixture of 37b (0.94 g, 1.8 mmol), sodium benzenesulfinate (2.18 g, 13 mmol) and N,N-dimethylformamide (31.8 g) was stirred at room temperature for 12 h, then in a 40° C. bath for ca.6 h until all educt was converted as shown by TLC (1:4 ethyl acetate-hexane). The solution was equilibrated with 1:1 ethyl acetate-hexane (120 mL) and 1:1 brine-water (45 mL). The organic layer was washed with water (4×25 mL) brine (10 mL), then dried and evaporated to leave a colorless oil, 1.0317 g. This material was flash-chromatographed using a stepwise gradient (1:9, 1:6, 1:3 ethyl acetate-hexane) to give a colorless oil, 0.930 g, 96%: 300 MHz ¹H NMR δ −0.02 (3H, s), 0.00 (3H, s), 0.87 (9H, s), 0.88 (3H, s), 1.12 (1H, m), 1.20 (6H, s), 1.2-1.8 (18H, m), 1.81 (1H, m), 3.09 (2H, m), 3.97 (1H, brs), 7.59 (3H, m), 7.91 2H, m).

### [1R,3aR,4S,7aR]-1-(1(S)-Benzenesulfonylmethyl-5-methyl-5-trimethylsilanyloxy-hexyl)-4-(tert-butyl-dimethyl-silanyloxy)-7a-methyl-octahydro-indene (39)

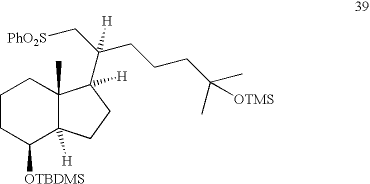

1-(Trimethylsilyl)imidazole (1 mL) was added to a solution of 38 (0.8 g) in cyclohexane (10 mL) and stirred overnight then flash-chromatographed using a stepwise gradient of hexane, 1:39 and 1:19 ethyl acetate-hexane. The elution was monitored by TLC (1:4 ethyl acetate-hexane) leading to 39 as a colorless syrup, 0.7915 g: 300 MHz ¹H NMR δ 0.00 (3H, s), 0.02 (3H, s), 0.12 (9H, s), 0.90 (12H, s, t-butyl+7a-Me), 1.16 (1H, m), 1.20 (6H, s), 1.2-1.6 (15H, m), 1.66-1.86 (3H, m), 3.10 (2H, m), 4.00 (1H, brs), 7.56-7.70 (3H, m), 7.93 (2H, m).

### [1R,3aR,4S,7aR]-6(R)-[4-(tert-Butyl-dimethyl-silanyloxy)-7a-methyl-octahydro-inden-1-yl]-2,10-dimethyl-undecane-2,3(R),10-triol (40)

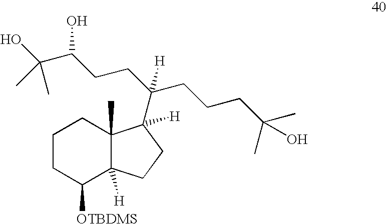

A solution of 39 (0.7513 g, 1.23 mmol) and diol (0.508 g, 1.85 mmol) in tetrahydrofuran (28 mL) was cooled to −35° C. then 2.5 M butyllithium in hexane (2.75 mL) was added dropwise. The temperature was allowed to rise to −20° C. and maintained at that temperature for 6 h or until the educt was consumed. Reaction progress was monitored by TLC (1:4 ethyl acetate-hexane) exhibiting the educt (Rf 0.71) and the two epimeric diols (Rf 0.09 and 0.12). Toward the end of the reaction period the temperature was increased briefly to 0° C., lowered again to −10, then saturated ammonium chloride (25 mL) was added followed by ethyl acetate (50 mL) and enough water to dissolve the precipitated salts. The resulting aqueous phase was extracted with ethyl acetate (15 mL). The combined extracts were washed with brine (15 mL), dried and evaporated. The resulting syrup was flash-chromatographed using a stepwise gradient of 1:9, 1:6, 1:4 and 1:1 ethyl acetate-hexane to give 39a as a colorless syrup, 0.8586 g. This material was dissolved in a mixture of tetrahydrofuran (30 mL) and methanol (18 mL), then 5% sodium amalgam (20 g) was added. The reductive de-sulfonylation was complete after stirring of the mixture for 14 h. Progress of the reaction was monitored by TLC (1:1 ethyl acetate-hexane) which showed the disappearance of the epimeric diols (Rf 0.63 and 0.74) and the generation of 40a (Rf 0.79) and the partially de-silylated analog 40 (Rf 0.16). The mixture was diluted with methanol (20 mL), stirred for 3 min, then ice (20 g) was added, stirred for 2 min and the supernatant decanted into a mixture containing saturated ammonium chloride (50 mL). The residue was repeatedly washed with small amounts of tetrahydrofuran that was also added to the salt solution, which was then equilibrated with ethyl acetate (80 mL). The aqueous layer was re-extracted once with ethyl acetate (20 mL), the combined extracts were washed with brine (10 mL) then dried and evaporated. The resulting colorless oil containing both 40a and 40 was dissolved in 10 mL of a 1 N oxalic acid solution in methanol (prepared from the dihydrate) effecting the selective hydrolysis of the trimethylsilyl ether within minutes. Calcium carbonate (1 g) was added and the suspension stirred overnight, then filtered. The solution was evaporated and the resulting residue flash-chromatographed using a stepwise gradient of 1:4, 1:2, 1:1 and 2:1 ethyl acetate-hexane giving a residue of the triol 40 that crystallized in very fine branching needles from acetonitrile, 0.45 g: mp 94-95° C., [α]_{D}+44.1° (methanol, c 0.37); 400 MHz ¹H NMR δ −0.005 (3H, s), 0.007 (3H, s), 0.89 (9H, s), 0.92 (3H, s), 1.15 (1H, m), 1.16 (3H, s), 1.21 (9H, s), 1.2-1.6 (19H, m), 1.67 (1H, m), 1.79 (2H, m), 1.90 (2H, m), 2.06 (1H, m), 3.31 (1H, brd, J=10 Hz), 4.00 (1H, brs), LR-ES(−) m/z: 533 (M+Cl), 497 (M−H); HR-ES(+): Calcd for C₂₉H₅₈O₄Si+Na: 521.3996. found: 521.4003. Anal Calcd for C₂₉H₅₈O₄Si: C, 69.82; H, 11.72. found: C, 69.97; H, 11.65.

### [1R,3aR,4S,7aR]-6(R)-(4-Hydroxy-7a-methyl-octahydro-inden-1-yl)-2,10-dimethyl-undecane-2,3(R),10-triol (41)

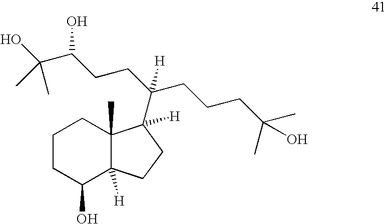

A stirred solution of the triol 40 (0.4626 g, 0.927 mmol) in acetonitrile (10 mL) and dioxane (0.7 mL) was cooled to 10° C. and a fluorosilicic acid solution (2 mL) was added dropwise. The cooling bath was removed, the 2-phase system further diluted with acetonitrile (2 mL) then stirred at room temperature for 3¼ h. The disappearance of educt was monitored by TLC (ethyl acetate). The mixture was equilibrated with water (10 mL) and ethyl acetate (30 mL). The aqueous phase was re-extracted with ethyl acetate (2×20 mL), the combined extracts were washed with water (5 mL) and brine (10 mL), then 1:1 brine-saturated sodium hydrogen carbonate solution and dried. The residue was purified by flash-chromatography using a step-wise gradient from 1:1 to 2:1 ethyl acetate-hexane and neat ethyl acetate to give a residue that was taken up in 1:1 dichloromethane-hexane, filtered and evaporated to furnish amorphous solids, 0.3039 g (85%): [α]_{D}+42.6° (methanol, c 0.48); ¹H NMR (DMSO-d₆): δ 0.87 (3H, s), 0.97 (3H, s), 1.02 (3H, s), 1.04 (6H, s), 1.1-1.4 (18H, m), 1.5-1.8 (4H, m), 1.84 (1H, m), 2.99 (1H, dd, J=6 and 10 Hz), 3.87 (1H, brs), 4.02 (1H, s, OH), 4.05 (1H, s, OH), 4.16 (1H, d, OH, J=3.6 Hz), 4.20 (1H, d, OH, J=6.4 Hz); LR-ES(+): m/z 384 (M), 383 (M−H); HR-ES(+): Calcd for (M+Na) 407.3132. found: 407.3134.

### [1R,3aR,4S,7aR]-1-{5-Hydroxy-5-methyl-1(R)-[2-(2,2,5,5-tetramethyl-[1,3]dioxolan-4(R)-yl)-ethyl]-hexyl}-7a-methyl-octahydro-inden-4-ol (42)

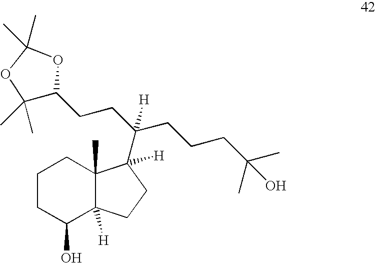

A solution of the tetraol 40 (0.2966 g, 0.771 mmol) and pyridinium tosylate (100 mg) in acetone (8 mL) and 2,2-dimethoxypropane (8 mL) was kept at room temperature for 12 h. TLC analysis (ethyl acetate) showed the absence of educt (Rf 0.21) and two new spots with Rf 0.82 and 0.71, the former the expected 42 and the latter assumed to be the methylacetal. The reaction mixture was diluted with water (5 mL) and stirred for 10 min. At that time only the spot with higher Rf value was observed. The mixture was neutralized with sodium hydrogen carbonate (0.5 g) then equilibrated with ethyl acetate (50 mL) and brine (5 mL). The organic layer was washed with water (5 mL) and brine (5 mL) then dried and evaporated to leave a sticky residue (0.324 g) that was used directly in the next step: 300 MHz ¹H NMR: δ 0.94 (3H, s), 1.10 (3H, s), 1.20 (1H, m), 1.22 (6H, s), 1.25 (3H, s), 1.34 (3H, s), 1.41 (3H, s), 1.2-1.65 (20H, m), 1.78-1.86 (3H, m), 1.93 (1H, m), 3.62 (1H, dd, J=4.6 and 8.3 Hz), 4.08 (1H, brs).

### [1R,3aR,4S,7aR]-Acetic acid 1-{5-hydroxy-5-methyl-1(R)-[2-(2,2,5,5-tetramethyl-[1,3]dioxolan-4(R)-yl)-ethyl]-hexyl}-7a-methyl-octahydro-inden-4-yl ester (43)

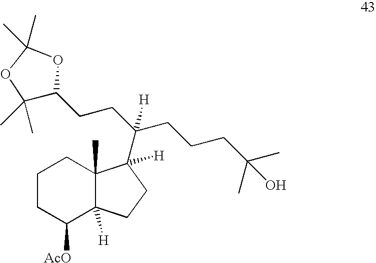

The residue obtained above was dissolved in pyridine (6.9 g) and further diluted with acetic anhydride (3.41 g). The mixture was allowed to stand at room temperature for 24 h, then in a 35° C. bath for ca. 10 h until the educt was no longer detectable (TLC, ethyl acetate). The mixture was diluted with toluene and evaporated. The residue was purified by flash chromatography (1:4 ethyl acetate-hexane) to give 43 as colorless syrup, 0.3452 g, 97%: ¹H NMR: δ 0.89 (3H, s), 1.10 (3H, s), 1.20 (1H, m), 1.22 (6H, s), 1.25 (3H, s), 1.33 (3H, s), 1.41 (3H, s), 1.25-1.6 (19H, m), 1.72 (1H, m), 1.82 (2H, m), 1.95 (1H, m), 2.05 (3H, s), 3.63 (1H, dd, J=4.4 and 8.4 Hz), 5.15 (1H, brs); LR-FAB(+) m/z 467 (M+H), 465 (M−H), 451 (M-Me).

### [1R,3aR,4S,7aR]-Acetic acid 1-[4(R),5-dihydroxy-1(R)-(4-hydroxy-4-methyl-pentyl)-5-methyl-hexyl]-7a-methyl-octahydro-inden-4-yl ester (44)

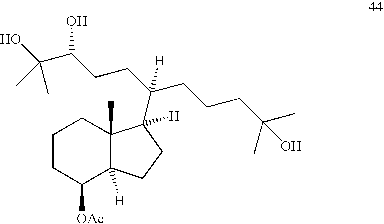

A solution of 43 (0.334 g, 0.716 mmol) in 80% acetic acid (2 mL) was kept in a 68° C. bath. TLC (ethyl acetate, Rf 0.33) monitored the progress of the hydrolysis. The educt was no longer detectable after 2.5 h. The mixture was evaporated then co-evaporated with a small amount of toluene to leave a colorless film (0.303 g) that was used directly in the next step: 300 MHz ¹H NMR: δ 0.89 (3H, s), 1.17 (3H, s), 1.22 (6H, s), 1.56 (3H, s), 1.1-1.6 (21H, m), 1.6-2.0 (5H, m), 2.04 (3H, s), 3.32 (1H, brd, J=10 Hz), 5.15 (1H, brs).

### [1R,3aR,4S,7aR]-Acetic acid 1-[4(R)-[dimethyl-(1,1,2-trimethyl-propyl)-silanyloxy]-5-hydroxy-1(R)-(4-hydroxy-4-methyl-pentyl)-5-methyl-hexyl]-7a-methyl-octahydro-inden-4-yl ester (45)

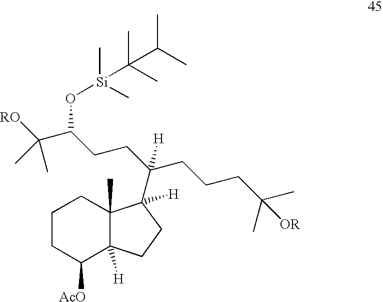

A solution of the triol 44 (0.30 g), imidazole (0.68 g, 10 mmol) and dimethylthexylsilyl chloride (1.34 g, 7.5 mmol) in N,N-dimethylformamide (6 g) was kept at room temperature. After 48 h 4-(N,N-dimethylamino)pyridine (15 mg) was added and the mixture stirred for an additional 24 h. Reaction progress was monitored by TLC (ethyl acetate; 24, Rf 0.83; 25a, Rf 0.38). The mixture was diluted with water (2 mL), stirred for 10 min then distributed between ethyl acetate (45 mL) and water (20 mL). The aqueous layer was extracted once with ethyl acetate (10 mL). The combined organic phases were washed with water (4×12 mL) and brine (8 mL) then dried and evaporated. The residual oil was purified by flash-chromatography using a stepwise gradient of 1:9 and 1:4 ethyl acetate-hexane to give 45 as colorless syrup. A small amount of unreacted educt (80 mg) was eluted with ethyl acetate. The syrupy 45 was used directly in the next step: 400 MHz ¹H NMR: δ 0.13 (3H, s), 0.14 (3H, s), 0.87 (6H, s), 0.91 (9H, m), 1.10 (1H, m), 1.14 (3H, s), 1.15 (3H, s), 1.21 (6H, s), 1.1-1.6 (19H, m), 1.6-1.9 (5H, m), 1.94 (1H, brd, J=12.8 Hz), 2.05 (3H, s), 3.38 (1H, brs), 5.15 (1H, brs).

### [1R,3aR,4S,7aR]-Acetic acid 1-[4(R)-[dimethyl-(1,1,2-trimethyl-propyl)-silanyloxy]-5-methyl-1(R)-(4-methyl-4-trimethylsilanyloxy-pentyl)-5-trimethylsilanyloxy-hexyl]-7a-methyl-octahydro-inden-4-yl ester (46)

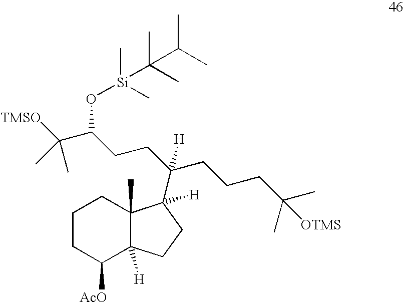

1-(Trimethylsilyl)imidazole (0.90 mL, 6.1 mmol) was added to a solution of 45 (0.2929 mg) in cyclohexane (6 mL) and stirred for 12 h, then flash-chromatographed (1:79 ethyl acetate-hexane) to yield 46 as colorless syrup (0.3372 g). The elution was monitored by TLC (1:4 ethyl acetate-hexane) leading to 46 as a colorless syrup, 0.7915 g: ¹H NMR δ 0.074 (3H, s), 0.096 (3H, s), 0.103 (9H, s), 0.106 (9H, s), 0.82 (1H, m), 0.83 (6H, s), 0.88 (9H, m), 1.32 (3H, s), 1.20 (9H, s), 1.15-1.6 (17H, m), 1.6-1.9 (5H, m), 1.97 (1H, brd, J=12.8 Hz), 2.05 (3H, s), 3.27 (1H, m), 5.15 (1H, brs); LR-FAB(+) m/z: 712 (M), 711 (M−H), 697 (M-Me), 653 (M-AcO), 627 (M-C₆H₁₃).

### [1R,3aR,4S,7aR]-1-[4(R)-[Dimethyl-(1,1,2-trimethyl-propyl)-silanyloxy]-5-methyl-1(R)-(4-methyl-4-trimethylsilanyloxy-pentyl)-5-trimethylsilanyloxy-hexyl]-7a-methyl-octahydro-inden-4-ol (47)

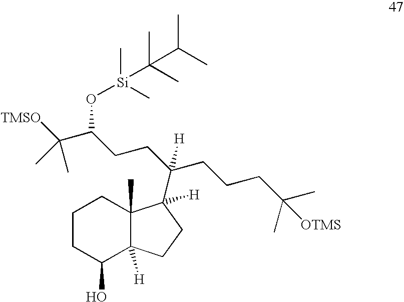

A stirred solution of 46 (0.335 mg, 0.47 mmol) in tetrahydrofuran (15 mL) was cooled in an ice-bath and a 1 M solution of lithium aluminum hydride in tetrahydrofuran (2 mL) was added dropwise. TLC (1:9 ethyl acetate-hexane) showed complete conversion 25b (Rf 0.61) to 26 (Rf 0.29) after 1.5 h. A 2 M sodium hydroxide solution (14 drops) was added, followed by water (0.5 mL) and ethyl acetate (30 mL). A small amount of Celite was added and, after stirring for 15 min, the liquid layer was filtered off. The solid residue was rinsed repeatedly with ethyl acetate and the combined liquid phases evaporated to leave a colorless syrup, that was taken up in hexane, filtered and evaporated to yield 26 (0.335 g) that was used without further purification: ¹H NMR δ: 0.075 (3H, s), 0.10 (21H, brs), 0.82 (1H, m), 0.84 (6H, s), 0.89 (6H, m), 0.93 (3H, s), 1.13 (3H, s), 1.20 (9H, s), 1.2-1.6 (16H, m), 1.6-1.7 (2H, m), 1.82 (3H, m), 1.95 (1H, brd, J=12.4 Hz), 3.27 (1H, m), 4.08 (1H, brs); LR-FAB(+) m/z: 585 (M-C₆H₁₃), 481 (M-TMSO); HR-ES(+) m/z: Calcd for C₃₇H₇₈O₄Si₃+Na: 693.5100. found: 693.5100.

### [1R,3aR,7aR]-1-[4(R)-[Dimethyl-(1,1,2-trimethyl-propyl)-silanyloxy]-5-methyl-1(R)-(4-methyl-4-trimethylsilanyloxy-pentyl)-5-trimethylsilanyloxy-hexyl]-7a-methyl-octahydro-inden-4-one (48)

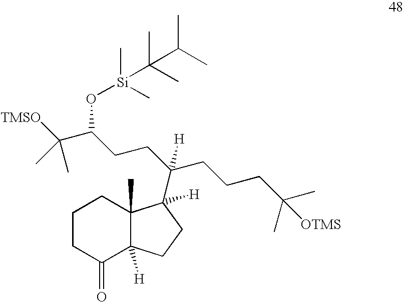

Celite (0.6 g) was added to a stirred solution of 47 (0.310 g, 0.462 mmol) in dichloromethane (14 mL) followed by pyridinium dichromate (0.700 g, 1.86 mmol). The conversion of 47 (Rf 0.54) to the ketone 27 (Rf 0.76) was followed by TLC (1:4 ethyl acetate-hexane). The mixture was diluted with cyclohexane after 4.5 h then filtered trough a layer of silica gel. Filtrate and ether washes were combined and evaporated. The residue was flash-chromatographed (1:39 ethyl acetate-hexane) to give 27 as a colorless syrup, 0.2988 g, 96.6%: ¹H NMR δ: 0.078 (3H, s), 0.097 (3H, s), 0.107 (18H, s), 0.64 (3H, s), 0.81 (1H, m), 0.84 (6H, s), 0.89 (6H, m), 1.134 (3H, s), 1.201 (3H, s), 1.207 (3H, s), 1.211 (3H, s), 1.3-1.6 (14H, m), 1.6-1.7 (3H, m), 1.88 (1H, m), 2.04 (2H, m), 2.2-2.32 (2H, m), 2.46 (1H, dd, J=7.5 and 11.5 Hz), 3.28 (1H, m); LR-FAB(+) m/z: 583 (M-C₆H₁₃), 479 (M-OTMS); HR-ES(+) m/z: Calcd for C₃₇H₇₆O₄Si₃+Na: 691.4943. found: 691.4949.

### [1R,3aR,7aR,4E]-4-{2(Z)-[3(S),5(R)-Bis-(tert-butyl-dimethyl-silanyloxy)-2-methylene-cyclohexylidene]-ethylidene}-7a-methyl-1-[5-methyl-1(R)-(4-methyl-4-trimethylsilanyloxy-pentyl)-4(R)-[dimethyl-(1,1,2-trimethyl-propyl)-silanyloxy]-5-trimethylsilanyloxy-hexyl]-octahydro-indene (49)

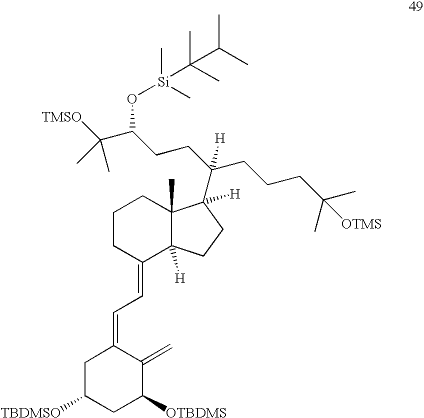

A solution of 2.5-M butyllithium in hexane (0.17 mL) was added to a solution of 28 in tetrahydrofuran (2 mL) at −70° C. to produce a deep cherry-red color of the yield. After 10 min a solution of ketone 27 (0.1415 g, 0.211 mmol) in tetrahydrofuran (2 mL) was added dropwise over a 15 min period. The reaction was quenched after 4 h by the addition of pH 7 phosphate buffer (2 mL). The temperature was allowed to increase to 0° C. then hexane (30 mL) was added. The aqueous layer was re-extracted with hexane (15 mL). The combined extracts were washed with of brine (5 mL), dried and evaporated to give a colorless oil that was purified by flash-chromatography (1:100 ethyl acetate-hexane) to yield 49 as colorless syrup, 0.155 g, 71%: ¹H NMR δ: 0.068 (15H, m), 0.103 (12H, s), 0.107 (9H, s), 0.53 (3H, s), 0.82 (1H, m), 0.84 (6H, s), 0.88 (18H, m), 0.89 (6H, m), 1.14 (3H, m), 1.20 (9H, s), 12-1.9 (22H, m), 1.97 (2H, m), 2.22 (1H, dd, J=7.5 an 13 Hz), 2.45 (1H, brd, J=13 Hz), 2.83 (1H, brd, J=13 Hz), 3.28 (1H, m), 4.20 (1H, m), 4.38 (1H, m), 4.87 (1H, d, J=2 Hz), 5.18 (1H, d, J=2 Hz), 6.02 (1H, d, J=11.4 Hz, 6.24 (1H, d, J=11.4 Hz); LR-FAB(+) m/z 1033 (M+H), 1032 (M), 1031 (M−H), 901 (M-TBDMS).

### Synthesis of 1,25-Dihydroxy-21-(2R,3-dihydroxy-3-methyl-butyl)-20R-Cholecalciferol (33)

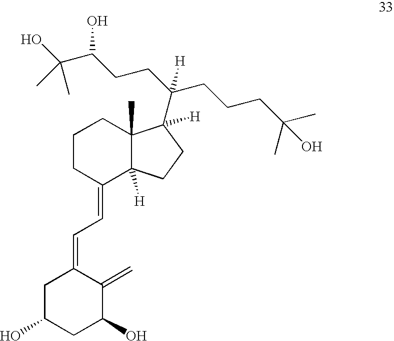

The residue of 49 (0.153 g, 0.148 mmol), as obtained in the previous experiment, was dissolved in a 1 M solution of tetrabutylammonium fluoride (3.5 mL). TLC (ethyl acetate) monitored reaction progress. Thus, the solution was diluted with brine (5 mL) after 24 h, stirred for 5 min then equilibrated with ethyl acetate (35 mL) and water (15 mL). The aqueous layer was re-extracted once with ethyl acetate (15 mL). The combined organic layers were washed with water (5×10 mL), once with brine (5 mL) then dried and evaporated. The residue was purified by flash chromatography using a stepwise gradient of ethyl acetate and 1:100 methanol-ethyl acetate furnishing 33 as colorless, microcrystalline material from methyl formate-pentane, 70 mg, 91%: [α]_{D}+34.3° (methanol, c 0.51); ¹H NMR (DMSO-d₆) δ: 0.051 (3H, s), 0.98 (3H, s), 1.03 (3H, s), 1.05 (6H, s), 1.0-1.6 (17H, m), 1.64 (3H, m), 1.80 (2H, m), 1.90 (1H, d, J=11.7 Hz), 1.97 (1H, dd, J=J=9.8 Hz), 2.16 (1H, dd, J=5.9 and J=13.7 Hz), 2.36 (1H, brd), 2.79 (1H, brd), 3.00 (1H, dd, J=5 and 10 Hz), 3.99 (1H, brs), 4.01 (1H, s, OH), 4.04 (1H, s, OH), 4.54 (1H, OH, d, J=3.9 Hz), 4.76 (1H, brs), 4.87 (1H, OH, d, J=4.9 Hz), 5.22 (1H, brs), 5.99 (1H, d, J=10.7 Hz), 6.19 (1H, d, J=10.7 Hz); LR-ES(+) m/z: 519 (M+H), 518 (M), 517 (M−H), 501 (M-OH); HR-ES(+) calcd for C₃₂H₅₄O₅+Na: 541.3863. found 541.3870; UVₘₐₓ (ε): 213 (13554), 241 sh (12801), 265 (16029) nm.

### Synthetic Example 42

### Synthesis of 1,25-Dihydroxy-21 (2R,3-dihydroxy-3-methyl-butyl)-20S-Cholecalciferol (50)

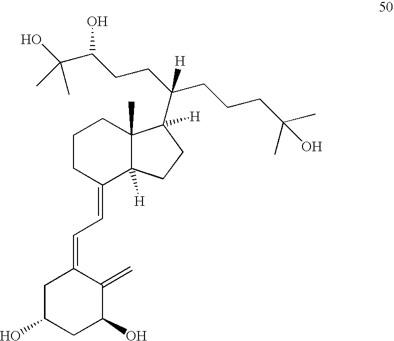

[1R,3aR,4S,7aR]-7-Benzenesulfonyl-6(R)-[4-(tert-butyl-dimethyl-silanyloxy)-7a-methyl-octahydro-inden-1-yl]-2-methyl-heptan-2-ol (51)

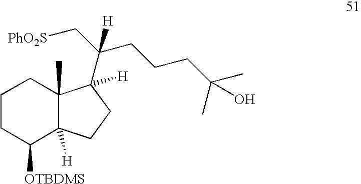

A solution of 36 and sodium benzenesulfonate (0.263 g, 1.6 mmol) in N,N-dimethyl formamide (5 mL) was stirred in a 77° C. bath for 3 h. The solution was equilibrated with 1:1 ethyl acetate-hexane (25 mL) and the organic layer washed with water (5×10 mL), dried and evaporated. The residue was flash-chromatographed with a stepwise gradient of 1:9, 1:4, and 1:3 ethyl acetate-hexane to furnish the sulfone as a colorless syrup: ¹H NMR δ −0.02 (3H, s), 0.005 (3H, s), 0.79 (3H, s), 0.87 (9H, s), 1.12 (1H, m), 1.19 (6H, s), 1.12 (1H, m), 1.20 (6H, s), 1.2-1.8 (18H, m), 2.08 (1H, m), 3.09 (1H, dd, J=9.3 and 14.5 Hz), 3.31 (1H, dd, J=3 and 14.5 Hz), 3.97 (1H, brs), 7.58 (3H, m), 7.66 (1H, m), 7.91 2H, m); LR-ES(+) m/z: 600 (M+Na+MeCN), 559 (M+Na); LR-ES(−) m/z: 536 (M), 535 (M−H); HR-ES(+): Calcd for C₃₀H₅₂O₄SSi+Na 559.3248. found 559.3253.

### [1R,3aR,4S,7aR]-1-(1(R)-Benzenesulfonylmethyl-5-methyl-5-trimethylsilanyloxy-hexyl)-4-(tert-butyl-dimethyl-silanyloxy)-7a-methyl-octahydro-indene (52)

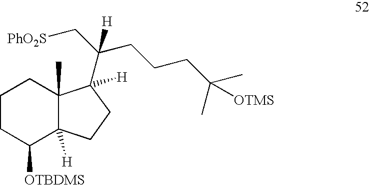

1-(Trimethylsilyl)imidazole (0.146 mL) was added to a solution of 51 (0.145 g, 0.27 mmol) in cyclohexane (2 mL). After 17 h the product was purified by flash chromatography using a stepwise gradient of 1:79 and 1:39 ethyl acetate-hexane to give 52 as colorless residue, 0.157 g 0.258 mmol, TLC (1:9 ethyl acetate-hexane) Rf 0.14. 300 MHz ¹H NMR: δ −0.02 (3H, s), 0.00 (3H, s), 0.87 (12H, s), 1.12 (1H, m), 1.17 (6H, s), 1.2-1.6 (15H, m), 1.6-1.9 (3H, m), 3.08 (2H, m), 3.97 (1H, brs), 7.53-7.70 (3H, m), 7.90 (2H, d, J=7 Hz).

### [1R,3aR,4S,7aR]-5(R,S)-Benzenesulfonyl-6(R)-[4-(tert-butyl-dimethyl-silanyloxy)-7a-methyl-octahydro-inden-1-yl]-2,10-dimethyl-10-trimethylsilanyloxy-undecane-2,3(R)-diol (53)

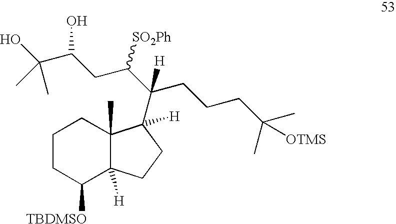

A solution of 152 (0.2589, 0.425 mmol) and diol (0.176 g, 0.638 mmol) in tetrahydrofuran (9 mL) was cooled to −25° C. and 1.6 M butyllithium in hexane (1.4 mL) was added. The temperature was raised to −20° C. and maintained for 3 h then at −10° C. for 2.5 h and 0° C. for 10 min. The mixture was cooled again to −10° C., saturated ammonium chloride solution (5 mL) was added, then equilibrated with ethyl acetate (50 mL) and enough water to dissolve precipitated salts. The aqueous layer was re-extracted with ethyl acetate (15 mL), the combined extracts were dried and evaporated and the residue purified by flash chromatography using a stepwise gradient of 1:6, 1:4, and 1:1 ethyl acetate-hexane to produce 53 as a colorless syrup, 0.212 g, 70%: 300 MHz ¹H NMR: δ 0.00 (3H, s), 0.017 (3H, s), 0.12 (9H, s), 0.81 (3H, s), 0.89 (9H, s), 1.16 (1H, m), 1.19 (12H, m), 1.1-1.6 (20H, m), 1.6-1.8 (2H, m), 3.10 (1H, dd, J=8.4 and 14.7 Hz), 3.30 (1H, m), 3.99 (1H, brs), 7.61 (2H, m), 7.67 (1H, m), 7.93 (2H, m).

### [1R,3aR,4S,7aR]-6(S)-[4-(tert-Butyl-dimethyl-silanyloxy)-7a-methyl-octahydro-inden-1-yl]-2,10-dimethyl-10-trimethylsilanyloxy-undecane-2,3(R)-diol (54)

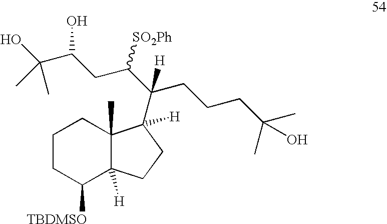

Compound 53 (0.186 mg, 0.262 mmol) was dissolved in 0.5 M oxalic acid dihydrate in methanol (2.5 mL). The solution was stirred for 15 min then calcium carbonate was added (0.5 g) and the suspension stirred overnight then filtered. The filtrate was evaporated to give 54 as a white foam, 0.188 g, 98%: TLC (1:1 ethyl acetate-hexane) Rf 0.06. This material was used in the next step without further purification.

### [1R,3aR,4S,7aR]-6(S)-[4-(tert-Butyl-dimethyl-silanyloxy)-7a-methyl-octahydro-inden-1-yl]-2,10-dimethyl-undecane-2,3(R),10-triol (triol 55)

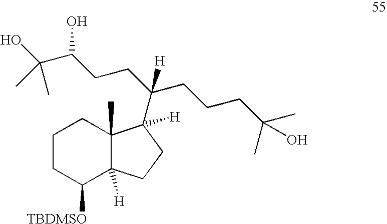

Sodium amalgam (5% sodium, 10.8 g) was added to a vigorously stirred solution of 54 (0.426 g, 0.667 mmol) in a mixture of tetrahydrofuran (15 mL) and methanol (9 mL). The suspension was stirred for 24 h and the reaction monitored by TLC (1:1 ethyl acetate-hexane0 to observe the production of 55 (Rf 0.17). The mixture was diluted with methanol (3 mL), stirred for 5 min then further diluted with water (10 mL), stirred for 2 min and decanted into saturated ammonium chloride solution (25 mL). The aqueous layer was extracted with ethyl acetate (2×20 mL). The combined extracts were washed with pH 7 phosphate buffer (5 mL) then brine (10 mL), dried and evaporated. The residue was purified by flash-chromatography using a stepwise gradient of 1:1 and 2:1 ethyl acetate-hexane to provide 55 as a colorless syrup, 0.244 g, 73%: ¹H NMR: δ-0.006 (3H, s), 0.006 (3H, s), 0.86 (9H, s), 0.92 (3H, s), 1.11 (1H, m), 1.15 (3H, s), 1.21 (9H, s), 1.2-1.75 (21H, m), 1.7-1.85 (3H, m), 1.90 (1H, m), 3.29 (1H, brd), 3.99 (1H, brs); LR-ES(+) m/z: 521 (M+Na), 481 (M-OH); LR-ES(−): m/z 544: (M+CH₂O₂), 543 (M−H+CH₂O₂), 533 (M−Cl); HR-ES(+) m/z: Calcd for C₂₉H₅₈O₄Si+Na: 521.3996. found 521.3999.

### [1R,3aR,4S,7aR]-6(S)-(4-Hydroxy-7a-methyl-octahydro-inden-1-yl)-2,10-dimethyl-undecane-2,3(R),10-triol (56)

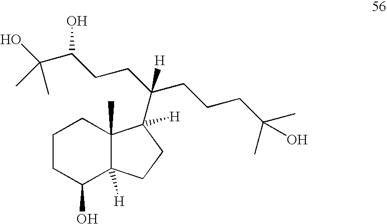

An aqueous fluorosilicic acid solution (3 mL) was added to a stirred solution of 55 (0.240 g, 0.481 mmol) in acetonitrile (12 mL). TLC (ethyl acetate) monitored the reaction. After 2.5 h compound 56 (Rf 0.37) was the predominating species, produced at the expense of less polar 55. The mixture was equilibrated with ethyl acetate and water (10 mL), the aqueous layer was re-extracted with water (2×10 mL) and the combined extracts were washed with water (6 mL) and brine (2×10 mL) then dried and evaporated. The colorless residue was flash-chromatographed using a stepwise gradient of 1:2, 1:1 and 2:1 ethyl acetate-hexane to elute some unreacted 55, followed by 56, obtained as colorless syrup, 0.147 g, 79%: ¹H NMR: 0.94 (3H, s), 1.12 (1H, m), 1.15 (3H, s), 1.21 (9H, s), 1.15-1.7 (20H, m), 1.7-1.9 (5H, m), 1.96 (1H, brd), 3.29 (1H, d, J=9.6 Hz), 4.08 (1H, brs); LR-ES(+): m/z 448: (M+Na+MeCN), 407 (M+Na); LR-ES(−): m/z 419 (M+Cl); HR-ES(+) m/z: Calcd for C₂₃H₄₄O₄+Na: 407.3132. found 407.3135.

### [1R,3aR,4S,7aR]-1-(5-Hydroxy-1(S)-{2-[2-(4-methoxy-phenyl)-5,5-dimethyl-[1,3]dioxolan-4(R)-yl]-ethyl}-5-methyl-hexyl)-7a-methyl-octahydro-inden-4-ol (57)

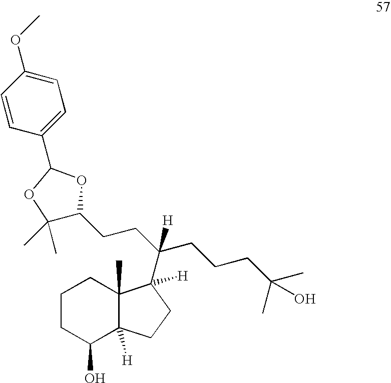

4-Methoxybenzaldehyde dimethyl acetal (60 μL, 0.35 mmol) was added to a solution of 56 (81.2 mg, 0.211 mmol) in dichloromethane (2 mL), followed by a solution (0.2 mL) containing pyridinium tosylate (200 mg) in dichloromethane (10 mL). Reaction progress was followed by TLC (1:2 ethyl acetate-hexane) which showed 4-methoxybenzaldehyde dimethyl acetal (Rf 0.80), 4-methoxybenzaldehyde (Rf 0.65), educt 56 (Rf 0.42) and product 57 (Rf 0.26). After 5¾ h the mixture was stirred for 15 min with saturated sodium hydrogencarbonate solution (5 mL) then equilibrated with ethyl acetate (25 mL). The organic layer was washed with brine (5 mL), dried and evaporated. The residue was flash-chromatographed using a stepwise gradient of 1:3 and 1:2 ethyl acetate-hexane to yield 57 as colorless syrup, 0.106 mg (100%): ¹H NMR: 0.94 (3H, s), 1.19, 1.21 (6H, s each, Me₂COH), 1.23, 1.35 and 1.24, 1.37 (6H, s each, major and minor 5,5-dimethyloxolane diastereomer), 1.1-1.7 (18H, m), 1.7-1.9 (5H, m), 1.9-2.0 (2H, m), 3.65 (1H, m), 3.81 (3H, s), 4.08 (1H, brs), 5.78 and 5.96 (1H, s each, major and minor acetal diastereomer), 6.89 (2H, m), 7.41 (2H, m).

### [1R,3aR,7aR]-1-(5-Hydroxy-1(S)-{2-[2-(4-methoxy-phenyl)-5,5-dimethyl-[1,3]dioxolan-4(R)-yl]-ethyl}-5-methyl-hexyl)-7a-methyl-octahydro-inden-4-one (58)

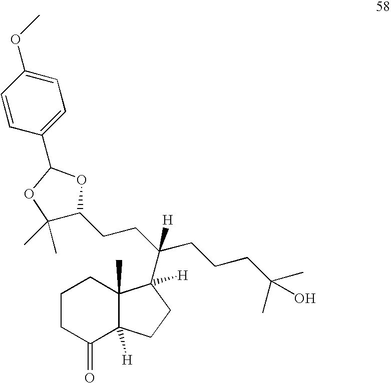

Pyridinium dichromate (230 mg, 0.61 mmol) was added to a stirred mixture containing 57 (0.0838, 0.167 mmol), Celite (185 mg), and dichloromethane (4 mL). The conversion of 57 (Rf 0.31) to 58 (Rf 0.42) was monitored by TLC (1:25 methanol-chloroform) The mixture was diluted with dichloromethane (10 mL) after 2.5 h, then filtered through a layer of silica gel. Filtrate and washings (1:1 dichloromethane-ethyl acetate) were evaporated and the residue chromatographed (1:4 ethyl acetate-hexane) to give ketone 58, 0.0763 g, 91%: ¹H NMR: 0.63 (3H, s), 1.19, 1.21 and 1.23 (6H, s each, Me₂COH), 1.25, 1.36, 1.38 (6H, m,s,s,5,5-dimethyloxolane diastereomer), 1.1-1.9 (18H, m), 1.9-2.1 (3H, m), 2.1-2.4 (2H, m), 2.45 (1H, m), 3.66 (1H, m), 3.802 and 3.805 (3H, s each), 5.78 and 5.95 (1H, s each, major and minor acetal diastereomer), 6.89 (2H, m), 7.39 (2H, m).

### [1R,3aR,7aR]-1-[4(R),5-Dihydroxy-1(S)-(4-hydroxy-4-methyl-pentyl)-5-methyl-hexyl]-7a-methyl-octahydro-inden-4-one (59)

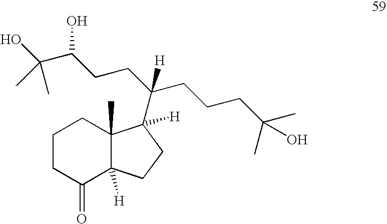

The ketone 58 was stirred in a 1 N oxalic acid solution in 90% methanol. The mixture became homogeneous after a few min. TLC (ethyl acetate) suggested complete reaction after 75 min (Rf 0.24 for 59). Thus, calcium carbonate (0.60 g) was added and the suspension stirred overnight, then filtered. The filtrate was evaporated and flash-chromatographed using a stepwise gradient of 4:1:5 dichloromethane-ethyl acetate-hexane, 1:1 ethyl acetate-hexane, and neat ethyl acetate produce 59 as a colorless residue, 0.060 mg, 94%: ¹H NMR: 0.5 (3H, s), 1.17 (3H, s), 1.22 (6H, s), 1.23 (3H, s), 1.2-1.21 (23H, m), 2.15-2.35 (2H, m), 2.45 (1H, dd, J=7 and 11 Hz), 3.30, 1H, brd).

### [1R,3aR,7aR]-7a-Methyl-1-[5-methyl-1(S)-(4-methyl-4-trimethylsilanyloxy-pentyl)-4(R),5-bis-trimethylsilanyloxy-hexyl]-octahydro-inden-4-one (60)

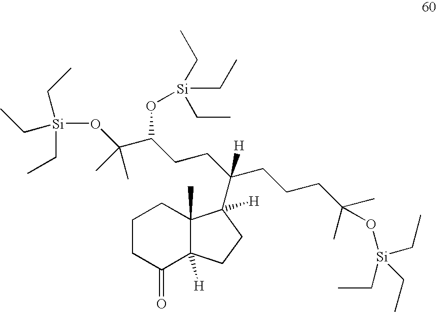

A mixture of 59 (0.055 g, 0.143 mmol), imidazole, (14.9 mg, 1.69 mmol), N,N-dimethylpyridine (6 mg), triethylchlorosilane (0.168 mL, 1 mmol) and N,N-dimethylformamide (1.5 mL) was stirred for 17 h. The reaction was followed by TLC (1:4 ethyl acetate-hexane) and showed rapid conversion to the disilyl intermediate (Rf 0.47). Further reaction proceeded smoothly overnight to give the fully silylated 60 (Rf 0.90). The solution was equilibrated with water (3 mL), equilibrated with ethyl acetate (20 mL), the ethyl acetate layer was washed with water (3×4 mL), dried and evaporated. The residue was flash-chromatographed using a stepwise gradient of hexane and 1:100 ethyl acetate-hexane to yield 60 as a colorless syrup, 0.0813 g, 78.4%: ¹H NMR δ 0.55-0.64 (21H, m), 0.92-0.97 (27H, m), 1.12 (3H, s), 1.18 (3H, s), 1.19 (3H, s), 1.21 (3H, s), 1.1-1.7 (18H, m), 1.9-2.15 (2H, m), 2.15-2.35 (2H, m), 2.43 (1H, dd, J=7.7 and 11 Hz), 3.30 (1H, dd, J=3 and 8.4 Hz).

### [1R,3aR,7aR,4E]-4-{2(Z)-[3(S),5(R)-Bis-(tert-butyl-dimethyl-silanyloxy)-2-methylene-cyclohexylidene]-ethylidene}-7a-methyl-1-[5-methyl-1(S)-(4-methyl-4-trimethylsilanyloxy-pentyl)-4(R),5-bis-triethylsilanyloxy-hexyl]-octahydro-indene (61)

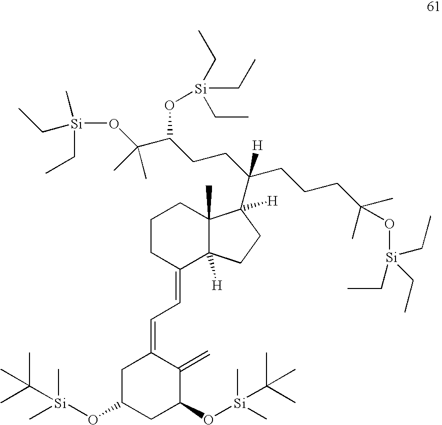

A solution of 1.6 M butyllithium in hexane (0.14 mL) was added to a solution of phosphine (0.1308 g, 0.224 mmol) in tetrahydrofuran (1.5 mL) at −70° C. After 10 min a solution of ketone 60 (0.0813 g, 0.112 mmol) in tetrahydrofuran (1.5 mL) was added dropwise over a 15 min period. The yield color had faded after 3 h so that pH 7 phosphate buffer (2 mL) was added and the temperature allowed to increase to 0° C. The mixture was equilibrated with hexane (30 mL), the organic layer was washed with brine (5 mL), dried and evaporated to give a colorless oil that was purified by flash-chromatography (1:100 ethyl acetate-hexane). Only the band with Rf 0.33 (TLC 1:39 ethyl acetate-hexane) was collected. Evaporation of those fractions gave 61 as colorless syrup, 0.070 g, 57%: ¹H NMR δ 0.06 (12H, brs), 0.53-0.64 (21H, m), 0.88 (18H, s), 0.92-0.97 (27H, m), 1.11 (3H, s), 1.177 (3H, s), 1.184 (3H, s), 1.195 (3H, s), 1-1.9 (22H, m), 1.98 (2H, m), 2.22 (1H, m), 2.45 (1H, m), 2.83 (1H, brd, J=13 Hz, 3.27 (1H, d, J=6 Hz), 4.19 (1H, m), 4.38 (1H, m), 4.87 (1H, brs), 5.18 (1H, brs), 6.02 (1H, d, J=11 Hz), 6.24 (1H, d, J=11 Hz).

### Synthesis of 1,25-Dihydroxy-21(2R,3-dihydroxy-3-methyl-butyl)-20S-Cholecalciferol (50)

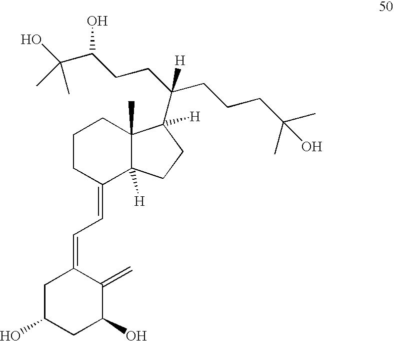

The deprotection reaction of 61 (0.068 g, 0.06238 mmol) in 1M solution of tetrabutylammonium fluoride in tetrahydrofuran, followed by TLC (ethyl acetate), gradually proceeded to give 50 (Rf 0.19). The mixture was diluted with brine (5 mL) after 25 h, stirred for 5 min the equilibrated with ethyl acetate (35 mL) and water (15 mL). The aqueous layer was re-extracted once with ethyl acetate (35 mL), the combined extracts were washed with water (5×10 mL) and brine (5 mL) then dried and evaporated. The residue was flash-chromatographed using a linear gradient of 1:1 and 2:1 ethyl acetate-hexane, and 2:98 methanol-ethyl acetate to give a residue that was taken up in methyl formate and evaporated to a white foam, 30 mg, 93%: [α]_{D}+29.3° (methanol, c 0.34); MHz ¹H NMR δ: 0.55 (3H, s), 1.16 (3H, s), 1.21 (9H, s), 1.1-1.75 (22H, m), 1.80 (2H, m), 1.9-2.1 (5H, m), 2.31 (1H, dd, J=7 and 13 Hz), 2.60 (1H, brd), 284 (1H, m), 3.29 (1H, d, J=9.5 Hz), 4.22 (1H, m), 4.43 (1H, m), 5.00 (1H, s), 5.33 (1H, s), 6.02 (1H, d, J=11 Hz), 6.02 (1H, d, J=11 Hz); LR-ES(−) m/z: 564 (M+H2CO2), 563 M−H+ H2CO2); HR-ES(+) calcd for C₃₂H₅₄O₅+Na: 541.3863. found 541.3854; UVₘₐₓ (ε): 211 (15017), 265 (15850), 204 sh (14127), 245 sh (13747) nm.

### Synthetic Example 43

### Synthesis of 1,25-Dihydroxy-21-(2R,3-dihydroxy-3-methyl-butyl)-20S-19-nor-cholecalciferol (62)

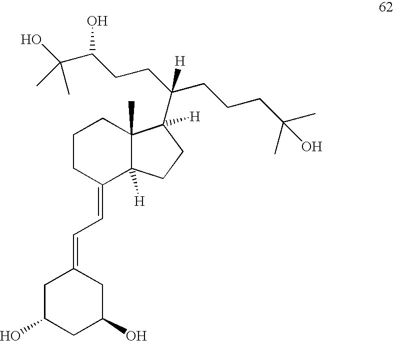

### [1R,3aR,7aR,4E]-4-{2(Z)-[3(S),5(R)-Bis-(tert-butyl-dimethyl-silanyloxy)-cyclohexylidene]-ethylidene}-7a-methyl-1-[5-methyl-1(S)-(4-methyl-4-triethylsilanyloxy-pentyl)-4(R),5-bis-triethylsilanyloxy-hexyl]-octahydro-indene (63)

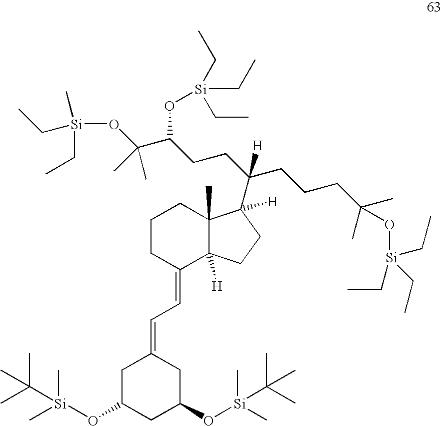

A solution of 1.6 M butyllithium in hexane was added to a solution of phosphine in tetrahydrofuran at −70° C. After 10 min a solution of ketone 60 from Example 2 in tetrahydrofuran was added dropwise over a 15 min period. After the yield color had faded, pH 7 phosphate buffer was added and the temperature allowed to increase to 0° C. The mixture was equilibrated with hexane, the organic layer was washed with brine, dried and evaporated to give a colorless oil that was purified by flash-chromatography (1:100 ethyl acetate-hexane) that gave 63.

### 1,25-Dihydroxy-21-(2R,3-dihydroxy-3-methyl-butyl)-20S-19-nor-cholecalciferol (62)

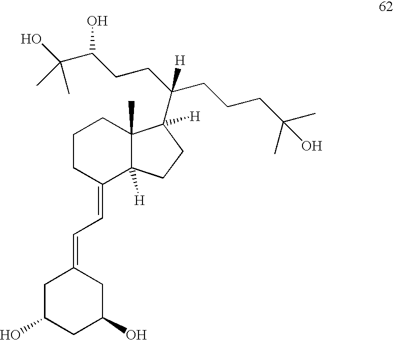

The deprotection reaction of 63 was carried out in 1M solution of tetrabutylammonium fluoride in tetrahydrofuran to give 62. The mixture was diluted with brine after 25 h, stirred for 5 min and then equilibrated with ethyl acetate and water. The aqueous layer was re-extracted once with ethyl acetate, the combined extracts were washed with water and brine, and then dried and evaporated. The residue was flash-chromatographed to give a residue that was taken up in methyl formate and evaporated to yield 62.

### Synthetic Example 44

### Synthesis of 1,25-dihydroxy-20S-21 (3-hydroxy-3-methyl butyl)-24-keto-19-nor-cholecalciferol (64)

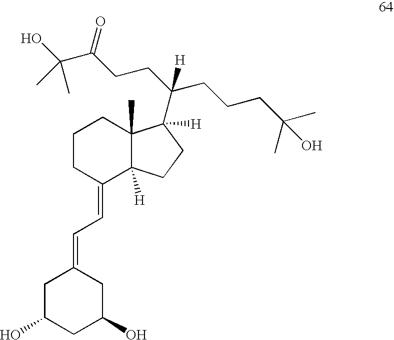

### (R)-6-[(1R,3aR,4S,7aR)-4-(tert-Butyl-dimethyl-silanyloxy)-7a-methyl-octahydro-inden-1-yl]-2-methyl-7-phenylsulfanyl-heptan-2-ol (65)

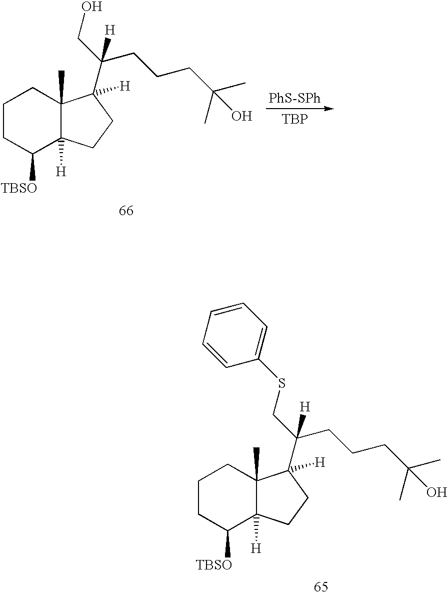

The reaction above was carried out as described in Tet Lett. 1975,17: 1409-12. Specifically, a 50 mL round-bottom flask was charged with 1.54 g (3.73 mmol) of (R)-2-[(1R,3aR,4S,7aR)-4-(tert-Butyldimethylsilanyloxy)-7a-methyloctahydroinden-1-yl]-6-methylheptane-1,6-diol (1) (Eur. J. Org. Chem. 2004, 1703-1713) and 2.45 g (11.2 mmol) of diphenylsulfide. The mixture was dissolved in 5 mL of pyridine and 2.27 g (11.2 mmol, 2.80 mL) of tributylphosphine was added. The mixture was stirred overnight and then diluted with 20 mL of toluene and evaporated. The residue was again taken up in toluene and evaporated, the remaining liquid chromatographed on silica gel using stepwise gradients of hexane, 1:39, 1:19 and 1:9 ethyl acetate-hexane to provide the title compound 65 as a syrup, 1.95 g.

### (R)-7-Benzenesulfonyl-6-[(1R,3aR,4S,7aR)-4-(tert-butyl-dimethyl-silanyloxy)-7a-methyl-octahydro-inden-1-yl]-2-methyl-heptan-2-ol (67) and (1R,3aR,4S,7aR)-1-((R)-1-Benzenesulfonylmethyl-5-methyl-5-triethylsilanyloxy-hexyl)-4-(tert-butyl-dimethyl-silanyloxy)-7a-methyl-octahydro-indene (68)

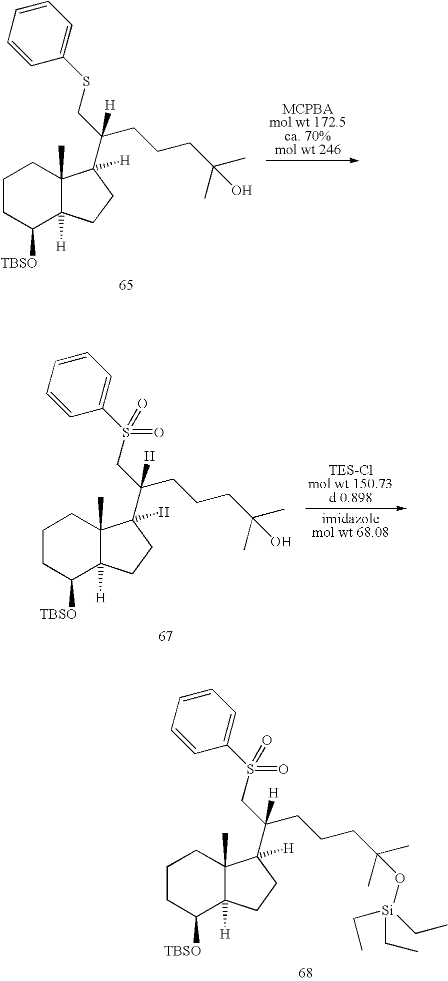

A 500-mL round-bottom flask containing 1.95 g (3.9 mmol) of the crude sulfide 65 was admixed with 84 g of dichloromethane (63 mL). The solution was stirred in an ice bath, then 2.77 g (11 mmol) of meta-chloroperbenzoic acid was added in one portion. The suspension was stirred in the ice bath for 40 min then at room temperature for 2 h. The reaction was monitored by TLC (1:19 methanol-dichloromethane). At the end of the reaction period, only one spot at Rf 0.45 observed. Then, 1.68 g (20 mmol) of solid sodium hydrogen carbonate was added to the suspension, the suspension was stirred for 10 min, then 30 mL of water was added in portions and vigorous stirring continued for 5 min to dissolve all solids. The mixture was further diluted with 40 mL of hexane, stirred for 30 min, transferred to a separatory funnel with 41.6 g of hexane. The lower layer was discarded and the upper one was washed with 25 mL of saturated sodium hydrogen carbonate solution, dried (sodium sulfate) and evaporated to give 3.48 g of 67. This material was triturated with hexane, filtered, and evaporated, to leave 67 as a cloudy syrup (2.81 g) that was used directly in the next step.

A 100-mL round bottom flask containing 2.81 g of 67 obtained above, was charged with 30 mL of N,N-dimethylformamide 1.43 g of (21 mmol) of imidazole and 1.75 mL of (10 mmol) of triethylsilyl chloride. The mixture was stirred for 17 h then diluted with 50 g of ice-water, stirred for 10 min, further diluted with 5 mL of brine and 60 mL of hexane. The aqueous layer was re-extracted with 20 mL of hexane, both extracts were combined, washed with 2×30 mL of water, dried, evaporated. This material contained a major spot with Rf 0.12 (1:39 ethyl acetate-hexane) and a minor spot with Rf 0.06. This material was chromatographed on silica gel using hexane, 1:100, 1:79, 1:39 and 1:19 ethyl acetate-hexane as stepwise gradients. The major band was eluted with 1:39 and 1:19 ethyl acetate-hexane to yield 1.83 g of 68.

### (R)-5-Benzenesulfonyl-6-[(1R,3aR,4S,7aR)-4-(tert-butyl-dimethyl-silanyloxy)-7a-methyl-octahydro-inden-1-yl]-10-methyl-2-(R)-methyl-10-triethylsilanyloxy-undecane-2,3-diol (69)

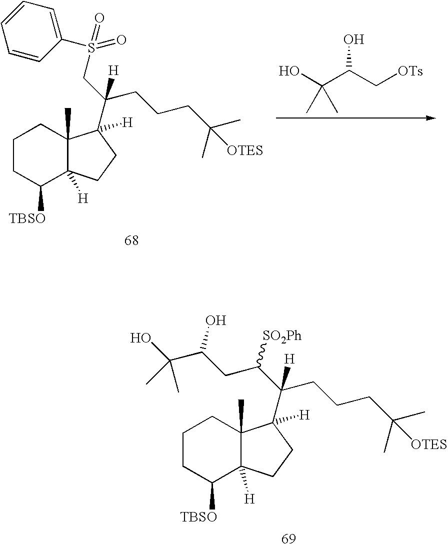

A 100-mL 3-neck round-bottom flask, equipped with magnetic stirrer, thermometer and Claisen adapter with rubber septum and nitrogen sweep, was charged with 1.7636 g of (2.708 mmol) of sulfone 68, 1.114 g of (4.062 mmol) tosylate, and 50 mL of tetrahydrofuran freshly distilled from benzophenone ketyl. This solution was cooled to −20° C. and 9.31 mL of a 1.6 M butyllithium solution in hexane was added dropwise at ≦−20° C. The temperature range between −10 and −20° C. was maintained for 5 h. The cooling bath was removed and 50 mL of saturated ammonium chloride solution added followed by 75 mL of ethyl acetate and enough water to dissolve all salts. The organic layer was washed with 15 mL of brine, dried, and evaporated to a colorless oil. This residue was chromatographed on silica gel using hexane, 1:9, 1:6, 1:4 and 1:3 ethyl acetate-hexane as stepwise gradients. The main band was eluted with 1:4 and 1:3 ethyl acetate-hexane to furnish 1.6872 g of compound 69 as colorless syrup.

### (S)-6-[(1R,3aR,4S,7aR)-4-(tert-Butyl-dimethyl-silanyloxy)-7a-methyl-octahydro-inden-1-yl]-10-methyl-2-(R)-methyl-10-triethylsilanyloxy-undecane-2,3-diol (70)

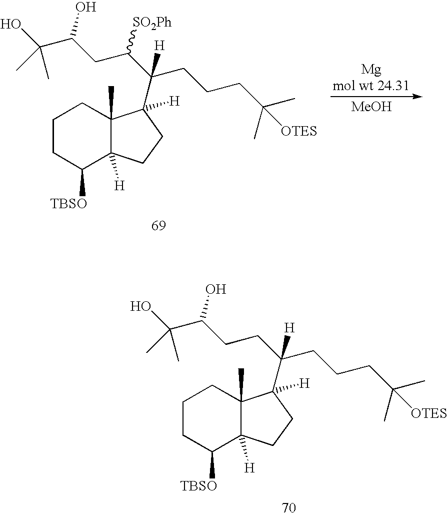

A 25-mL 2-neck round-bottom flask, equipped with magnetic stirrer, thermometer and Claisen adapter with rubber septum and nitrogen sweep, was charged with 1.6872 g (2.238 mmol) of sulfone 69 and 40 mL of methanol. Then 1.25 g (51.4 mmol) of magnesium was added to the stirred solution in two equal portions, in a 30 min time interval. The suspension was stirred for 70 min then another 0.17 g of magnesium and ca. 5 mL of methanol was added and stirring continued 1 h. The mixture was then diluted with 100 mL of hexane and 50 mL of 1 M sulfuric acid was added dropwise to give two liquid phases. The aqueous layer was neutral. The aqueous layer was re-extracted once with 25 mL of 1:1 dichloromethane-hexane. The organic layers were combined then washed once with 15 mL of brine, dried and evaporated. The resulting material was chromatographed on silica gel using hexane, 1:39, 1:19 and 1:9 ethyl acetate-hexane as stepwise gradients. The main band was eluted with 1:9 ethyl acetate-hexane to provide 1.2611 g of 70 as a colorless syrup.

### (S)-6-[(1R,3aR,4S,7aR)-4-(tert-Butyl-dimethyl-silanyloxy)-7a-methyl-octahydro-inden-1-yl]-2,10-dihydroxy-2,10-dimethyl-undecan-3-one (71)

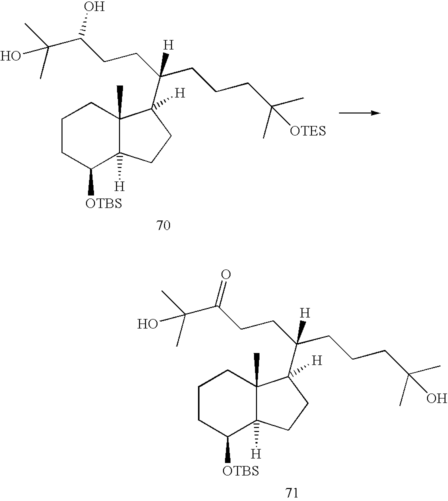

A 25-mL round-bottom flask, equipped with magnetic stirrer, thermometer, Claisen adapter with nitrogen sweep and rubber septum, was charged with 518 mg (3.88 mmol) of N-chlorosuccinimide and 11 mL of toluene. Stir for 5 min (not all dissolved), then cool to 0° C. and add 2.4 mL (4.8 mmol) of a 2M dimethyl sulfide solution in toluene. The mixture was stirred from 5 min then cooled to −30° C. and a solution of 0.7143 g (1.165 mmol) of the diol 70 in 4×1.5 mL of toluene was added dropwise at −30° C. Stirring was continued at this temperature for 1 h. The mixture was then allowed to warm to −10° C. during a 2 h time period then cooled to −17° C. and 3.20 mL (6.4 mmol) of 2 M triethylamine in toluene added dropwise. The mixture was stirred at −17 to −20° C. for 10 min then allowed to warm to room temperature slowly. The mixture was chromatographed on a silica gel column using hexane, 1:79, 1:39, 1:19, 1:9, 1:4, and 1:1 ethyl acetate-hexane as stepwise gradients. The major band was eluted with 1:1 ethyl acetate-hexane providing 0.3428 g of the compound 71 as solids.

### (S)-2,10-Dihydroxy-6-((1R,3aR,4S,7aR)-4-hydroxy-7a-methyl-octahydro-inden-1-yl)-2,10-dimethyl-undecan-3-one (72)

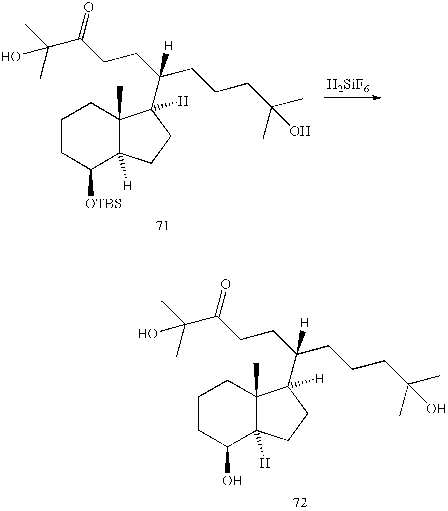

A 25-mL round-bottom flask, equipped with magnetic stirrer was charged with 0.3428 g (0.69 mmol) of the diol 71, was dissolved in 5 mL of acetonitrile then 1.25 mL of fluorosilicic acid solution. After 3 h, the mixture was distributed between 35 mL of ethyl acetate and 10 mL of water, the aqueous layer was re-extracted with 10 mL of ethyl acetate, the organic layers combined, washed with 2×5 mL of water, once with 5 mL of 1:1 brine-saturated sodium hydrogen carbonate solution, dried and evaporated. This material was chromatographed on silica gel using 1:4, 1:3, 1:2, and 1:1 as stepwise gradients furnishing 0.2085 g of the title compound 72.

### (1R,3aR,7aR)-1-[(S)-5-Hydroxy-1-(4-hydroxy-4-methyl-pentyl)-5-methyl-4-oxo-hexyl]-7a-methyl-octahydro-inden-4-one (73)

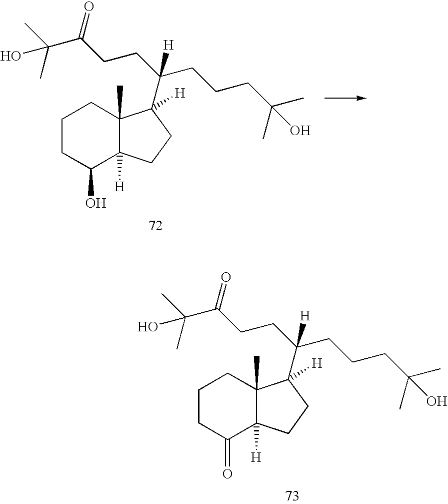

A 25-mL round bottom flask was charged with 0.2153 g (0.56 mmol) of 72, 5 mL of dichloromethane, and 0.20 g of Celite. To this stirred suspension was added, in on portion, 1.00 g (2.66 mmol) of pyridinium dichromate. The reaction stirred for 3 h and the progress was monitored by TLC (1:1 ethyl acetate-hexane). The reaction mixture was diluted with 5 mL of cyclohexane then filtered trough silica gel G. The column was eluted with dichloromethane followed by 1:1 ethyl acetate-hexane until no solute was detectable in the effluent. The effluent was evaporated and the colorless oil. This oil was then chromatographed on a silica gel using 1:4, 1:3, 1:2, 1:1 and 2:1 ethyl acetate-hexane as stepwise gradients to furnish 0.2077 g of the diketone 73.

### (1R,3aR,7aR)-7a-Methyl-1-[(S)-5-methyl-1-(4-methyl-4-trimethylsilanyloxy-pentyl)-4-oxo-5-trimethylsilanyloxy-hexyl]-octahydro-inden-4-one (74)

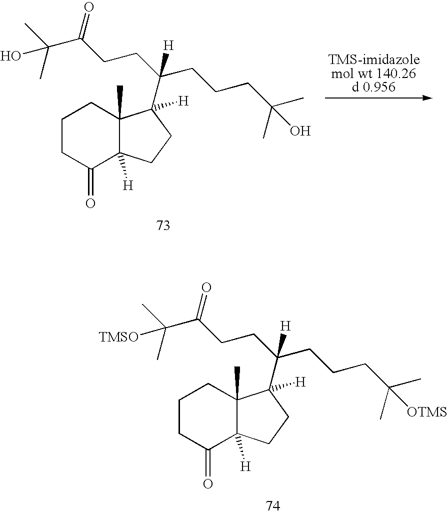

A 25-mL round bottom flask was charged with 0.2077 g (0.545 mmol) of the diketone 73. This material was dissolved in a mixture of 0.5 mL of tetrahydrofuran and 3 mL of cyclohexane. To the resulting mixture was added 0.30 mL (2.0 mmol) of TMS-imidazole. The reaction mixture was diluted with 3 mL of hexane after 10 h then concentrated and chromatographed on silica gel using hexane, 1:79, 1:39, 1:19 and ethyl acetate-hexane as stepwise gradients to provide 0.2381 g of 74 as a colorless oil.

### (S)-6-((1R,3aS,7aR)-4-{2-[(R)-3-((R)-tert-Butyldimethylsilanyloxy)-5-(tert-butyldimethylsilanyloxy)-cyclohexylidene]-ethylidene}-7a-methyloctahydroinden-1-yl)-2,10-dimethyl-2,10-bis-trimethylsilanyloxyundecan-3-one (75)

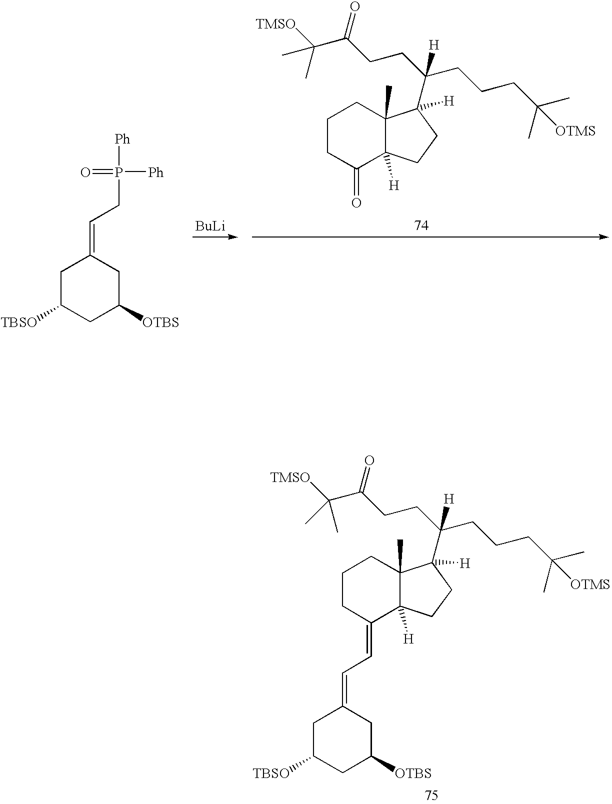

A 15-mL 3-neck pear-shaped flask, equipped with magnetic stirrer, thermometer and a Claisen adapter containing a nitrogen sweep and rubber septum, was charged with 0.2722 g (0.4768 mmol) of [2-[(3R,5R)-3,5-bis(tert-butyldimethylsilanyloxy)cyclohexylidene]ethyl]diphenylphosphine oxide and 2 mL of tetrahydrofuran. The solution was cooled to −70° C. and 0.30 mL of 1.6 M butyllithium in hexane was added. The deep red solution was stirred at that temperature for 10 min then 0.1261 g (0.240 mmol) of the diketone 74, dissolved in 2 mL of tetrahydrofuran was added, via syringe, dropwise over a 10 min period. After 3 h and 15 min, 5 mL of saturated ammonium chloride solution was added at −65° C., the mixture allowed to warm to 10° C. then distributed between 35 mL of hexane and 10 mL of water. The aqueous layer was re-extracted once with 10 mL of hexane, the combined layers washed with 5 ml of brine containing 2 mL of pH 7 buffer, then dried and evaporated. This material was chromatographed on a flash column, 15×150 mm using hexane and 1:100 ethyl acetate-hexane as stepwise gradients to yield 0.1572 g of the title compound 75 as a colorless syrup.

### 1,25-Dihydroxy-20S-21 (3-hydroxy-3-methyl-butyl)-24-keto-19-nor-cholecalciferol (64)

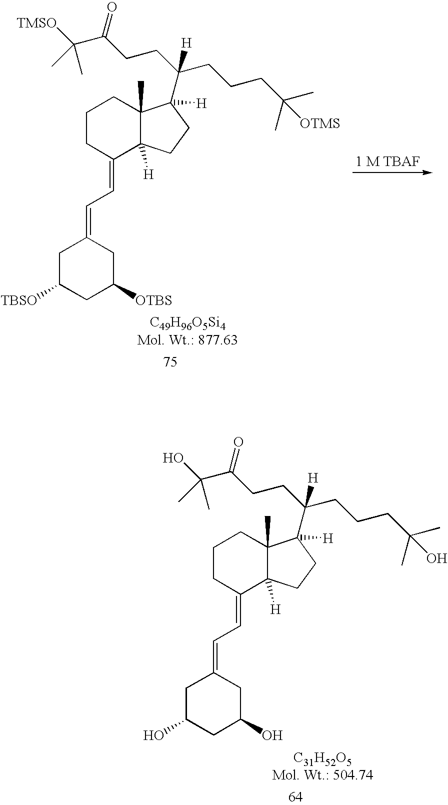

A 15-mL 3-neck round-bottom flask, equipped with magnetic stirrer, was charged with 155 mg (0.17 mmol) of tetrasilyl ether 75. This colorless residue was dissolved is 2 mL of a 1 M solution of tetrabutylammonium fluoride in tetrahydrofuran. After 43 h an additional 0.5 mL of 1 M solution of tetrabutylammonium fluoride solution was added and stirring continued for 5 h. The light-tan solution was the diluted with 5 mL of brine, stirred for 5 min and transferred to a separatory funnel with 50 mL of ethyl acetate and 5 mL of water then re-extraction with 5 mL of ethyl acetate. The organic layers were combined, washed with 5×10 mL of water, 10 mL of brine, dried and evaporated. The resulting residue was chromatographed on a 15×123 mm column using 2:3, 1:1, 2:1 ethyl acetate-hexane, and ethyl acetate as stepwise gradients to provide the 64 as a white solid (TLC, ethyl acetate, Rf 0.23) that was taken up in methyl formate, filtered and evaporated furnishing 0.0753 g of the title compound 64 as a solid substance.

### Synthetic Example 45

### Synthesis of 1,25-dihydroxy-20S-21 (3-hydroxy-3-methyl-butyl)-24-keto-cholecalciferol (76)

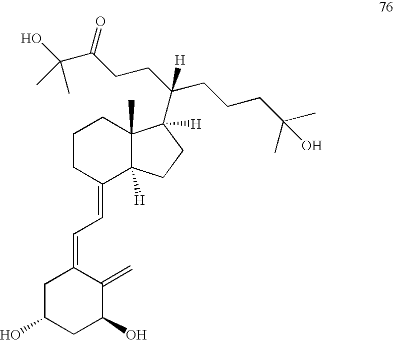

### (S)-6-{(1R,3aS,7aR)-4-[2-[(R)-3-(tert-Butyl-dimethyl-silanyloxy)-5-((S)-tert-butyl-dimethyl-silanyloxy)-2-methylene-cyclohexylidene]-eth-(E)-ylidene]-7a-methyl-octahydro-inden-1-yl}-2,10-dimethyl-2,10-bis-trimethylsilanyloxy-undecan-3-one

Compound 77 was prepared as described for 75 in Example 4 but by reacting 74 with [(2Z)-2-[(3S,5R)-3,5-bis(tert-butyldimethylsilanyloxy)methylenecyclohexylidene]-ethyl]diphenylphosphine oxide.

### 1,25-Dihydroxy-20S-21 (3-hydroxy-3-methyl-butyl)-24-keto-cholecalciferol (76)

Compound 76 was prepared from 77 by deprotecting 77 as described in Example 44 for 64.

### Synthetic Example 46

### Synthesis of 1α,25-Dihydroxy-16-ene-20-cyclopropyl-cholecalciferol (78) (Compound H)

Compound (78) was synthesized according to the following synthetic procedure.

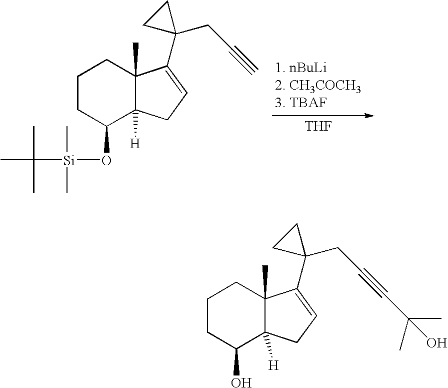

To a stirred solution of (3aR,4S,7aR)-1-{1-[4-(tert-Butyl-dimethyl-silanyloxy)-7a-methyl-3a,4,5,6,7,7a-hexahydro-3H-inden-1-yl])-cyclopropyl}ethynyl (1.0 g, 2.90 mmol) in tetrahydrofurane (15 mL) at −78° C. was added n-BuLi (2.72 mL, 4.35 mmol, 1.6M in hexane). After stirring at −78° C. for 1 h., acetone (2.5 mL, 34.6 mmol) was added and the stirring was continued for 2.5 h. NH₄Cl_{aq} was added (15 mL) and the mixture was stirred for 15 min at room temperature then extracted with AcOEt (2×50 mL). The combined extracts were washed with brine (50 mL) and dried over Na₂SO₄. The residue after evaporation of the solvent (2.4 g) was purified by FC (50 g, 10% AcOEt in hexane) to give (3aR,4S,7aR)-5-{1-[4-(tert-Butyl-dimethyl-silanyloxy)-7a-methyl-3a,4,5,6,7,7a-hexahydro-3H-inden-1-yl]-cyclopropyl}-2-methyl-pent-3-yn-2-ol (1.05 g, 2.61 mmol) which was treated with tetrabutylammonium fluoride (6 mL, 6 mmol, 1.0M in THF) and stirred at 65-75° C. for 48 h. The mixture was diluted with AcOEt (25 mL) and washed with water (5×25 mL), brine (25 mL). The combined aqueous washes were extracted with AcOEt (25 mL) and the combined organic extracts were dried over Na₂SO₄. The residue after evaporation of the solvent (1.19) was purified by FC (50 g, 20% AcOEt in hexane) to give the titled compound (0.75 g, 2.59 mmol, 90%). [α]³⁰_{D}=+2.7 c 0.75, CHCl₃. ¹H NMR (CDCl₃): 5.50 (1H, m), 4.18 (1H, m), 2.40 (2H, s), 2.35-1.16 (11H, m), 1.48 (6H, s), 1.20 (3H, s), 0.76-0.50 (4H, m); ¹³C NMR (CDCl₃): 156.39, 125.26, 86.39, 80.19, 69.21, 65.16, 55.14, 46.94, 35.79, 33.60, 31.67, 29.91, 27.22, 19.32, 19.19, 17.73, 10.94, 10.37;

MS HREI Calculated for C₂₂H₂₈O₂ M+ 288.2089. Observed M+ 288.2091.

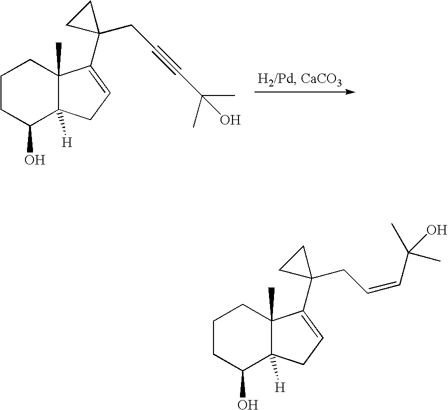

The mixture of (3aR,4S,7aR)-7a-Methyl-1-[1-(-4-hydroxy-4-methyl-pent-2-ynyl)-cyclopropyl]-3a,4,5,6,7,7a-hexahydro-3H-inden-4-ol (0.72 g, 2.50 mmol), ethyl acetate (10 mL), hexane (24 mL), absolute ethanol (0.9 mL), quinoline (47 μL) and Lindlar catalyst (156 mg, 5% Pd on CaCO₃) was hydrogenated at room temperature for 2 h. The reaction mixture was filtered through a celite pad and the pad was washed with AcOEt. The filtrates and the washes were combined and washed with 1 M HCl, NaHCO₃ and brine. After drying over Na₂SO₄ the solvent was evaporated and the residue (0.79 g) was purified by FC (45 g, 20% AcOEt in hexane) to give the titled compound (640 mg, 2.2 mmol, 88%).

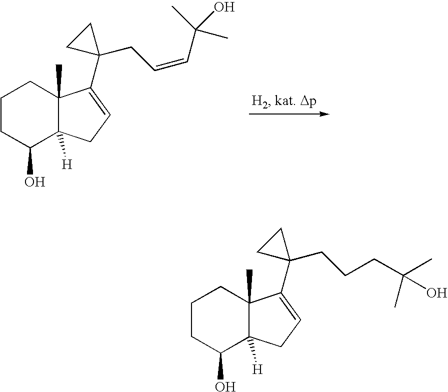

The mixture of (3aR,4S,7aR)-7a-Methyl-1-[1-(4-hydroxy-4-methyl-pent-2Z-enyl)-cyclopropyl]-3a,4,5,6,7,7a-hexahydro-3H-inden-4-ol (100 mg, 0.34 mmol), 1,4-bis(diphenyl-phosphino)butane 1,5 cyclooctadiene rhodium tetrafluoroborate (25 mg, 0.034 mmol), dichloromethane (5 mL) and one drop of mercury was hydrogenated using Paar apparatus at room temperature and 50 p.s.i. pressure for 3 h. The reaction mixture was filtered through Celite pad, which was then washed with ethyl acetate. The combine filtrates and washes were evaporated to dryness (110 mg) and purified by FC (10 g, 20% AcOEt in hexane) to give the titled compound (75 mg, 0.26 mmol, 75%). [α]³⁰_{D}=−8.5 c 0.65, CHCl₃. ¹H NMR (CDCl₃): 5.37 (1H, m,), 4.14 (1H, m), 2.37-1.16 (17H, m), 1.19 (6H, s), 1.18 (3H, s), 0.66-0.24 (4H, m);

MS HREI Calculated for C₁₉H₃₂O₂ M+H 292.2402. Observed M+H 292.2404.

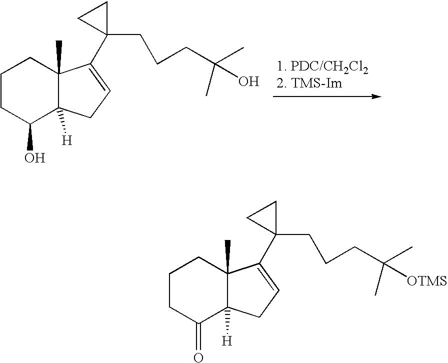

To a stirred suspension of (3aR,4S,7aR)-7a-Methyl-1-[1-(4-hydroxy-4-methyl-pentenyl)-cyclopropyl]-3a,4,5,6,7,7a-hexahydro-3H-inden-4-ol (440 mg, 1.50 mmol) and Celite (2.0 g) in dichloromethane (10 mL) at room temperature wad added pyridinium dichromate (1.13 g, 3.0 mmol). The resulting mixture was stirred for 5 h filtered through silica gel (10 g), and then silica gel pad was washed with 20% AcOEt in hexane. The combined filtrate and washes were evaporated, to give a crude (3aR,7aR)-7a-Methyl-1-[1-(4-hydroxy-4-methyl-pentenyl)-cyclopropyl]-3a,4,5,6,7,7a-hexahydro-3H-inden-4-one (426 mg, 1.47 mmol, 98%). To a stirred solution of (3aR,7aR)-7a-Methyl-1-[1-(4-hydroxy-4-methyl-pentenyl)-cyclopropyl]-3a,4,5,6,7,7a-hexahydro-3H-inden-4-one (424 mg, 1.47 mmol) in dichloromethane (10 mL) at room temperature was added trimethylsilyl-imidazole (0.44 mL, 3.0 mmol). The resulting mixture was stirred for 1.0 h filtered through silica gel (10 g) and the silica gel pad was washed with 10% AcOEt in hexane. Combined filtered and washes were evaporated to give the titled compound (460 mg, 1.27 mmol, 86%). [α]²⁹_{D}=−9.9 c 0.55, CHCl₃. ¹H NMR (CDCl₃): 5.33 (1H, dd, J=3.2, 1.5 Hz), 2.81 (1H, dd, J=10.7, 6.2 Hz), 2.44 (1H, ddd, J=15.6, 10.7, 1.5 Hz), 2.30-1.15 (13H, m) overlapping 2.03 (ddd, J=15.8, 6.4, 3.2 Hz), 1.18 (6H, s), 0.92 (3H, s), 0.66-0.28 (4H, m), 0.08 (9H, s); ¹³C NMR (CDCl₃): 211.08 (0), 155.32 (0), 124.77 (1), 73.98 (0), 64.32 (1), 53.91 (0), 44.70 (2), 40.45 (2), 38.12 (2), 34.70 (2), 29.86 (3), 29.80 (3), 26.80 (2), 24.07 (2), 22.28 (2), 21.24 (0), 18.35 (3), 12.60 (2), 10.64 (2), 2.63 (3); MS HRES Calculated for C₂₂H₃₈O₂Si M+ 362.2641. Observed M+ 362.2648.

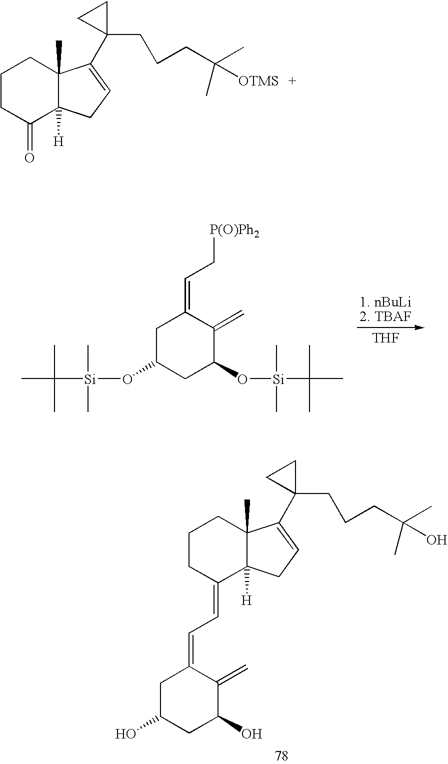

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (675 mg, 1.16 mmol) in tetrahydrofurane (8 mL) at −78° C. was added n-BuLi (0.73 mL, 1.17 mmol). The resulting mixture was stirred for 15 min and solution of (3aR,7aR)-7a-Methyl-1-[1-(4-methyl-4-trimethylsilanyloxy-pentyl)-cyclopropyl]-3a,4,5,6,7,7a-hexahydro-3H-inden-4-one (210 mg, 0.58 mmol, in tetrahydrofurane (2 mL) was added dropwise. The reaction mixture was stirred at −72° C. for 3.5 h diluted with hexane (35 mL) washed brine (30 mL) and dried over Na₂SO₄. The residue (850 mg) after evaporation of the solvent was purified by FC (15 g, 10% AcOEt in hexane) to give 1α,3β-Di(tert-Butyl-dimethyl-silanyloxy)-25-trimethylsilanyloxy-16-ene-20-cyclopropyl-cholecalciferol (382 mg, 0.53 mmol). To the 1α,3β-Di(tert-Butyl-dimethyl-silanyloxy)-25-trimethylsilanyloxy-16-ene-20-cyclopropyl-cholecalciferol (382 mg, 0.53 mmol) tetrabutylammonium fluoride (4 mL, 4 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (380 mg) after evaporation of the solvent was purified by FC (15 g, 50% AcOEt in hexane and AcOEt) to give the titled compound (78) (204 mg, 0.48 mmol, 83%). [α]²⁹_{D}=+16.1 c 0.36, EtOH. UV λmax (EtOH): 208 nm (ε 17024), 264 nm (ε 16028); ¹H NMR (CDCl₃): 6.37 (1H, d, J=11.3 Hz), 6.09 (1H, d, J=11.1 Hz), 5.33 (2H, m), 5.01 (1H, s), 4.44 (1H, m), 4.23 (1H, m), 2.80 (1H, m), 2.60 (1H, m), 2.38-1.08 (20H, m), 1.19 (6H, s), 0.79 (3H, s), 0.66-0.24 (4H, m); ¹³C NMR (CDCl₃): 157.07 (0), 147.62 (0), 142.49 (0), 133.00 (0), 124.90 (1), 124.73 (1), 117.19 (1), 111.64 (2), 71.10 (1), 70.70 (0), 66.88 (1), 59.53 (1), 50.28 (0), 45.19 (2), 43.85 (2), 42.86 (2), 38.13 (2), 35.59 (2), 29.27 (2), 29.14 (3), 28.65 (2), 23.57 (2), 22.62 (2), 21.29 (0), 17.84 (3), 12.74 (2), 10.30 (2); MS HRES Calculated for C₂₈H₄₂O₃ M+Na 449.3026. Observed M+Na 449.3023.

### Synthetic Example 47

### Synthesis of 1-alpha-fluoro-25-hydroxy-16,23E-diene-26,27-bishomo-20-epi-cholecalciferol (79) (Compound B)

Compound (79) is synthesized according to the following synthetic procedure.

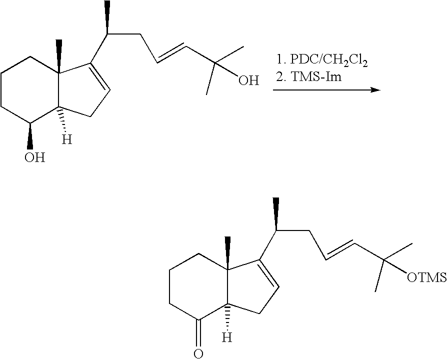

To a stirred suspension of 11-(5-Hydroxy-1,5-dimethyl-hex-3-enyl)-7a-methyl-3a,4,5,6,7,7a-hexahydro-3H-inden-4-ol and Celite in dichloromethane (10 mL) at room temperature is added pyridinium dichromate. The resulting mixture is stirred for 5 h filtered through silica gel, and then silica gel pad is washed with 20% AcOEt in hexane. The combined filtrate and washes are evaporated, to give a ketone. To a stirred solution of ketone in dichloromethane at room temperature is added trimethylsilyl-imidazole. The resulting mixture is stirred for 1.0 h filtered through silica gel and the silica gel pad is washed with 10% AcOEt in hexane. Combined filtered and washes are evaporated to give the titled compound.

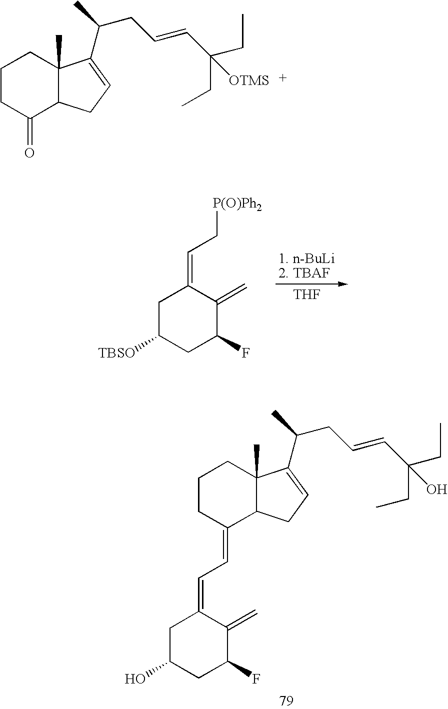

To a stirred solution of a tert-Butyl-{3-[2-(diphenyl-phosphinyl)-ethylidene]-5-fluoro-4-methylene-cyclohexyloxy}-dimethyl-silanein tetrahydrofurane at −78° C. is added n-BuLi. The resulting mixture is stirred for 15 min and solution of 1-(5-Ethyl-1-methyl-5-trimethylsilanyloxy-hept-3-enyl)-7a-methyl-3,3a,5,6,7,7a-hexahydro-inden-4-one in tetrahydrofurane is added dropwise. The reaction mixture is stirred at −78° C. for 3.5 h diluted with hexane washed brine and dried over Na₂SO₄. The residue after evaporation of the solvent was purified by FC (15 g, 10% AcOEt in hexane) to give the silylated compound. To the silylated compound, tetrabutylammonium fluoride is added, at room temperature. The mixture is stirred for 15 h. diluted with AcOEt (25 mL) and washed with water (5×20 mL), brine (20 mL) and dried over Na₂SO₄ The residue (380 mg) after evaporation of the solvent is purified by FC (15 g, 50% AcOEt in hexane and AcOEt) to give the titled compound (79).

### BIOLOGICAL EXAMPLES

### Biological Example 1

### Evaluation of Vitamin D3 Analogues (Compound A) in an In Vivo Model—Cyclophosphamide (CYP) Induced Chronic IC in Rats

The rat model of chemical cystitis induced by intraperitoneal injection of CYP has been Well accepted. CYP is used in clinical practice in the treatment of a number of malignant tumors. One of its metabolites, acrolein, is excreted in urine in large concentrations causing hemorrhagic cystitis associated with symptoms of urinary frequency, urgency and pelvic pain. The inflammatory process is characterized by changes in gross histology of bladder, increase in number and distribution of inflammatory cell infiltrates (mast cells, macrophage, PMNs), cyclo-oxygenase-2 expression and prostaglandin production, growth factor and cytokine production. The rat model of chemical cystitis closely resembles interstitial cystitis, a chronic, painful urinary bladder syndrome and has been used for the testing of therapeutic agents in the past.

This model was used to test the effects of oral administration of 1,25-dihydroxyvitamin D3 analogue in rats with CYP-induced cystitis. The effects of the treatment on the cystometric parameters in a conscious freely moving rat with CYP-induced cystitis were monitored. The following cystometric parameters were recorded in each animal:
bladder capacity
filling pressure (pressure at the beginning of the bladder filling)
threshold pressure (bladder pressure immediately prior to micturition)
micturition pressure (the maximal bladder pressure during micturition)
presence or absence of non-voiding bladder contractions (increases in bladder pressure of at least 10 cm H₂O without release of urine)
amplitude of non-voiding bladder contraction.

Animals: Wistar female rats, age 8 weeks, weighing 125-175 g were used. Two groups of animals had a tube implanted into the urinary bladder for intravesical pressure recording. Following recovery all animals received three intraperitoneal injections of CYP and subsequently were divided into the treatment and sham control groups.

Treatment group: Rats treated with oral 1,25-dihydroxyvitamin D3 analog (1,3-Di-O-acetyl-1,25-dihydroxy-16,23Z-diene-26,27-hexafluoro-19-nor-cholecalciferol) “Compound A” for 14 days (daily dose of 0.1 μg/kg)

Control group: Rats treated with oral vehicle (miglyol) in the dose identical to that delivered in the treatment group

Cystometry was performed 24 hours following the last dose of the drug or vehicle on awake freely moving animals.

Number of animals per group:

| | |
|---|---|
| Sham control animals | 4 |
| Treated animals | 3 |

Methods
Implantation of the Polyethylene Tubing into the Urinary Bladder:

A lower midline abdominal incision was performed under general inhalation anesthesia (isoflurine with O2) and polyethylene tubing (PE-50, Clay Adams, Parsippany, N.J.) with the end flared by heat was inserted into the dome of the bladder and secured in place with a 6-0 prolene purse string suture. The distal end of the tubing was heat-sealed, tunneled subcutaneously and externalized at the back of the neck, out of the animal's reach. Abdominal and neck incisions were closed with 4-0 nylon sutures.

Intraperitoneal Injection of Cyclophosphamide:

Following recovery (5 days) subject animals underwent three intraperitoneal injections of CYP (Sigma Chemical, St. Luis, Mo.; 75 mg/kg each, intraperitoneal) over the period of nine days. On the tenth day following the first CYP injection the sham control animals received the vehicle only, whereas the experimental group were treated with the 1,25-dihydroxyvitamin D3 analogue 1,3-Di-O-acetyl-1,25-dihydroxy-16,23Z-diene-26,27-hexafluoro-19-nor-cholecalciferol “Compound A” (delivered orally using gavage). Two weeks following the initiation of the treatment animals underwent a conscious cystometrogram to assess the function of the urinary bladder.

Cystometrogram

An animal was placed unrestrained in a cage and the catheter was connected via a T-tube to a pressure transducer (Grass® Model PT300, West Warwick, R.I.) and microinjection pump (Harvard Apparatus 22, South Natick, Mass.). A 0.9% saline solution was infused at room temperature into the bladder at a rate of 10 ml/hour. Intravesical pressure was recorded continuously using a Neurodata Acquisition System (Grass® Model 15, Astro-Med, Inc, West Warwick, R.I.). At least three reproducible micturition cycles were recorded after the initial stabilization period of 25-30 minutes.

Timeline of an Experiment:

| Procedure | Days |
|---|---|
| Acclimation period | 1-5 |
| Tube implantation + recovery period | 6-10 |
| CYP treatment (three doses of 75 mg/kg i.p. | 11-17 |
| every three days) | |
| Treatment (sham or active) | 18-31 |
| Cystometric evaluation | 32 |

Results

The data analysis is summarized in Tables 1 and 2 and FIG. 1 in which:

TABLE 1
cystometric parameters for the control group.

| | Bl. | | | | # | Amplitude |
|---|---|---|---|---|---|---|
| Rat | Cap. | FP | TP | MP | of NVBC | of NVBC |
| RB8 | 1.2 | 15 | 15 | 100 | 22 | 15 |
| | 1.2 | 13 | 18 | 100 | 14 | 14 |
| | 1.1 | 16 | 15 | 82 | 12 | 11 |
| RB10 | 0.7 | 30 | 40 | 110 | 26 | 25 |
| | 0.9 | 32 | 26 | 94 | 32 | 28 |
| | 0.6 | 26 | 26 | 108 | 35 | 16 |
| RB12 | 1.7 | 35 | 40 | 115 | 40 | 17 |
| | 1.7 | 25 | 30 | 125 | 35 | 14 |
| | 1.9 | 30 | 25 | 118 | 22 | 17 |
| RB14 | 1.3 | 16 | 16 | 104 | 10 | 10 |
| | 1.2 | 17 | 17 | 95 | 4 | 8 |
| | 1.1 | 19 | 21 | 92 | 9 | 18 |

TABLE 2
cystometnc parameters for the treatment group

| | Bl. | | | | # | amplitude |
|---|---|---|---|---|---|---|
| Rat | Cap. | FP | TP | MP | of NVBC | of NVBC |
| RB7 | 0.7 | 13 | 14 | 98 | 0 | 0 |
| | 0.7 | 14 | 14 | 97 | 0 | 0 |
| | 0.8 | 13 | 14 | 101 | 0 | 0 |
| RB13 | 1.4 | 14 | 15 | 104 | 8 | 11 |
| | 1.9 | 15 | 16 | 105 | 4 | 10 |
| | 1.3 | 14 | 17 | 97 | 8 | 11 |
| RB15 | 2.5 | 12 | 14 | 90 | 0 | 0 |
| | 1.3 | 11 | 12 | 100 | 0 | 0 |
| | 1.5 | 10 | 11 | 108 | 0 | 0 |

Changes were noted in a number of cystometric parameters. Dramatic reductions in both the number and amplitude of non-voiding bladder contractions were observed in the drug treated animals. Less pronounced but still statistically significant reductions in the filling and threshold pressures were also recorded. The treatment did not result in a change of the bladder capacity.

Bladder overactivity associated with chronic cystitis manifests itself in frequent contractions of the bladder wall associated with irritative often painful urinary symptoms. The fact that non-voiding bladder contractions were reduced both in their frequency and amplitude strongly suggest that if the effects on the bladder function in patients with interstitial cystitis will be similar, oral treatment with vitamin D3 analogues has a potential to relieve these debilitating symptoms. Reduction in filling and threshold pressures is significant from a clinical standpoint because the increased intravesical pressure associated with interstitial cystitis is a condition potentially jeopardizing the upper urinary tract.

### Biological Example 2

### Histological Analysis of Rat Bladders

Rat bladders from the experiments of Example 1 were fixed in formalin, embedded in paraffin and stained with hematoxylin and eosin by methods known in the art.

Histopathological examination was performed on at least 10 sections per bladder. Different inflammatory parameters were considered:

hemostasis

edema

infiltration of inflammatory cells (mostly lymphocytes and monocytes)

epithelial erosion

fibrosis

and were scored as follows: 0=normal without any sign of inflammation, 1=mild, 2=moderate, 3=severe, 4=severe signs diffused across all of the section.

Results

Tables 3 and 4 below show the effect of Compound A on histological score. Table 3 refers to vehicle treated animals and Table 4 to “Compound A” treated animals. Each inflammatory parameter was scored from 0 to 4, where 0 is normal and 4 the most severe symptom.

TABLE 3

| | | | | | EPITHELIAL | |
|---|---|---|---|---|---|---|
| RAT # | EDEMA | INFILTRATION | HEMOSTASIS | FIBROSIS | EROSION | TREATED |
| RB8 | 2 | 1 | 2 | 0 | 0 | MIGLYOL |
| RB10 | 1 | 1 | 1 | 1 | 2 | MIGYOL |
| RB12 | 0 | 3 | 1 | 3 | 2 | MIGYOL |
| RB14 | 4 | 4 | 3 | 0 | 0 | MIGYOL |
| MEAN | 1.75 | 2.25 | 1.75 | 1 | 1 | |
| STD | 1.71 | 1.5 | 0.96 | 1.41 | 1.15 | |

TABLE 4

| | | | | | EPITHELIAL | |
|---|---|---|---|---|---|---|
| RAT# | EDEMA | INFILTRATION | HEMOSTASIS | FIBROSIS | EROSION | TREATED |
| RB1 | 0.5 | 0.5 | 0 | 0 | 0 | Compound A |
| RB15 | 0 | 1 | 1 | 0 | 0 | Compound A |
| RB13 | 0 | 0.5 | 0.5 | 0 | 0 | Compound A |
| RB7 | 2 | 2 | 0.5 | 0 | 0 | Compound A |
| MEAN | 0.63 | 1 | 0.5 | 0 | 0 | |
| STD | 0.95 | 0.71 | 0.41 | 0 | 0 | |

FIG. 2 shows the effect of Compound A on the histological signs of inflammation in rat bladders, whilst FIG. 3 shows a histogram summarizing the histological score for each sign of inflammation.

The data of Examples 1 and 2 clearly demonstrate the utility of vitamin D3 analogues for treating the inflammatory component of interstitial cystitis as well as the consequent bladder overactivity characterizing interstitial cystitis.

### Biological Example 3

### Evaluation of Vitamin D3 Analogues (Compound B) in an In Vivo Model—Cyclophosphamide (CYP) Induced Chronic IC in Rats

Method

Implantation of the Polyethylene Tubing into the Urinary Bladder

Wistar rats (250 gr female weighing 125-175 g, age 8 weeks) were used. Under general inhalation anesthesia with 2% isoflurane, polyethylene tubing (PE-50, Clay Adams, Parsippany, N.J.) with the end flared by heat was inserted into the dome of the bladder and secured in place with a 6-0 nylon purse string suture. The distal end of the tubing was sealed, tunneled subcutaneously and externalized through a small incision at the back of the neck. The tubing was then coiled and buried subcutaneously.

Intraperitoneal Injection of Cyclophosphamide

Following recovery (5 days) subject animals underwent three intraperitoneal injections of CYP (Sigma Chemical, St. Louis, Mo.; 75 mg/kg each, intraperitoneal) over the period of nine days. On the tenth day following the first CYP injection the sham control animals received the vehicle only, whereas the experimental group were treated with the 1,25-dihydroxyvitamin D3 analogue 1,3-Di-O-acetyl-1,25-dihydroxy-16,23Z-diene-26,27-hexafluoro-19-nor-cholecalciferol “Compound A” (delivered orally using gavage). Two weeks following the initiation of the treatment animals underwent a conscious cystometrogram to assess the function of the urinary bladder.

Treatment

Treatment Group: 4-6 rats were treated with oral vitamin D₃ analogue (1-alpha-fluoro-25-hydroxy-16,23E-diene-26,27-bishomo-20-epi-cholecalciferol) Compound “B” continuously for 14 days (daily dose of either 30 or 75 ug/kg).

Control Group: 4 rats were treated with oral vehicle (miglyol) at a dose identical to that delivered in the treatment group.

An ethanol stock solution of Compound B (1 mg/ml) was dissolved in Miglyol vehicle at the appropriate concentration. Control animals received the vehicle containing the same amount of ethanol. Drug (or vehicle) treatment was carried out by daily gavage after weighing the animal. Drug solution was prepared calculating a final volume of 100 ul/10 grams body weight.

Cystometrogram

Following two weeks of treatment, animals were placed unrestrained in a cage and the distal end of the catheter was extracted from the subcutaneous pouch and connected via T-tube to a pressure transducer (Grass® Model PT300, West Warwick, R.I.) and microinjection pump (Harvard Apparatus 22, South Natick, Mass.). Saline solution (0.9%, room temperature) was infused into the bladder at a rate of 10 ml/h. Intravesical pressure was recorded continuously using a Neurodata Acquisition System (Grass®, Astro-Med, West Warwick, R.I.). At a minimum, three reproducible micturition cycles were recorded. The following cystometric parameters were recorded in each animal: filling pressure (FP: pressure at the beginning of the bladder filling), threshold pressure (TP: bladder pressure immediately prior to micturition), micturition pressure (MP: the maximal bladder pressure during micturition), presence or absence of non-voiding bladder contractions (NVBC: increases in bladder pressure of at least 10 cm H₂O without release of urine). The post-void residual (PVR) was measured by aspirating the residual urine remaining in the bladder after the last micturition or draining the bladder by gravity. Bladder capacity (BC) was calculated as the sum of voided volume and PVR. The bladder was harvested and its weight was recorded following euthanasia.

Calcemia Assessment

Serum calcemia was evaluated by a commercially available calorimetric assay (Calcium Dry-Fast, Sentinel CH. Italy). Briefly, 10 ul serum were added to 100 ul reaction solution prepared according to the manufacturer's procedure in a 96-wells plate. After 5 min incubation at RT samples were read at 570 nm with a spectrophotometer and the calcium levels were calculated in triplicate by using a standard reference as directed.

Results

TABLE 5
cystometric parameters (standard error
values are shown in brackets)

| | Bl. | FP (cm | TP (cm | MP (cm | # of | Amplitude |
|---|---|---|---|---|---|---|
| Group | Cap. | H₂O) | H₂O) | H₂O) | NVBC | of NVBC |
| Control | 1.2 | 22.8 | 24.1 | 103.6 | 21.8 | 16.8 |
| | (0.1) | (8.0) | (8.2) | (4.1) | (4.0) | (1.9) |
| Compound | 2.0 | 23.3 | 29.3 | 79.3 | 11.7 | 16.6 |
| B (30 ug/kg) | (0.6) | (10.6) | (12.6) | (26.8) | (16.6) | (9.7) |
| Compound | 2.1 | 26.1 | 36.3 | 90.5 | 8.3 | 13.6 |
| B (75 ug/kg) | (0.1) | (9.0) | (7.6) | (3.1) | (1.9) | (1.3) |

Changes were again noted in a number of cystometric parameters as a result of treatment with a vitamin D compound (in this case Compound B).

Non-voiding bladder contractions were significantly reduced in their frequency at both dosage levels (30 ug/kg, p<0.01; 75 ug/kg, p<0.005), see FIG. 4. The treatment with Compound B resulted in an increased bladder capacity at both dosage levels (30 ug/kg, p<0.01; 75 ug/kg, p<0.01) as shown in FIG. 5.

Marginal increase in the filling and threshold pressures were seen as a result of treatment with Compound B (not statistically significant). The amplitude of non-voiding bladder contractions was reduced by treatment with Compound B at the higher dose level (not statistically significant).

At both dosage levels of Compound B average serum calcium levels were only slightly but not significantly elevated, see FIG. 6 (the dotted line indicated the limit of normal range of 10.7 mg/dl in rats).

### Biological Example 4

### Evaluation of Vitamin D3 Analogues (Compound B) in an In Vivo Model—Allergic IC in Mice

Method

General

Mice (BALB/c, females 8 weeks old, weight 18-20 g, Charles River, Calco ITA) were sensitized by injecting 10 ug/mouse of chicken ovalbumin from Sigma (OVA, grade V) in the presence of 4 mg of alum (SERVA, Germany) by intraperitoneal injection, once a week for 4 weeks. This induces a sustained level of IgE detectable in serum.

One or two weeks after the last immunization boost, sensitized mice were anesthetized (1.5% Isoflurane) and then trans-urethrally catheterized (24 gauge, ¾ in.; Angiocath, Becton-Dickson). Slight digital pressure was applied the lower abdomen to drain the urine.

The urinary bladders were instilled with either 150 ul of either saline or OVA alone, (1 mg/150 ul) infused at a slow rate to avoid trauma and vesico-ureteral reflux and repeated twice within a 30-min interval with the syringe kept in place on the catheter for at least 30 min. Intravesical challenge was repeated 7-10 days after the first one with the same procedure.

Treatment Group: 10 mice were treated with oral vitamin D₃ analogue (1-alpha-fluoro-25-hydroxy-16,23Ediene-26,27-bishomo-20-epi-cholecalciferol) Compound “B” for 14 days (daily dose of either 30 or 75 ug/kg).

Control Group: 10 mice treated with oral vehicle (miglyol) in the dose identical to that delivered in the treatment group.

An ethanol stock solution of Compound B (1 mg/ml) was dissolved in Miglyol vehicle at the appropriate concentration. Control animals received the vehicle containing the same amount of ethanol. Drug (or vehicle) treatment was carried out by daily gavage after weighing the animal. Drug solution was prepared calculating a final volume of 100 ul/10 grams body weight, and treatment was started on the day of the first intravesical ovalbumin challenge, maintained daily, but discontinued over the weekends, for 12 days total treatment. FIG. 7 illustrates the timeline of the experiment.

ELISAS

Serum total IgE levels were measured by using a commercially available kit (BD Opteia, Mouse IgE ELISA Set Cat. No. 555248) and following manufacturer's instructions. Briefly, microwells were coated with 50 uL per well of Capture Antibody diluted in Coating Buffer (0.1 M Sodium Carbonate, pH 9.5 8.40 g NaHCO3, 3.56 g Na2CO3; q.s. to 1.0 L; pH to 9.5; freshly prepared or used within 7 days of preparation, stored at 2-8° C.). Recommended antibody coating dilution 1:250 as from lot-specific Instruction/Analysis Certificate. Plates were sealed and incubated overnight at 4° C. After washes (6× in PBS/tween 0.1%) plates were blocked with ≧200 μL/well PBS/10% FBS) and incubated at room temperature (RT) for 1 hour. 100 uL of each standard, sample, and control were pipetted into appropriate wells and plates were incubated for 2 hours at RT. After 6 washes 100 uL of prepared Detection Antibody+Avidin-HRP (1:250) reagent were dispensed to each well and let them stand for 1 hour at RT. After washes as above, 0.1 ml of Substrate Solution were added to the plates and further incubated (without plate sealer) for 30 minutes RT in the dark. After reaction stopping (H₂SO₄ 1M, 50 ul/well) absorbance at 450 nm was recorded within 30 minutes.

Ova-specific IgE were measured according to the following procedure. An anti-mouse IgE antibody (1 ug/ml Pharmingen, cat.) was coated in carbonate buffer and plates were incubated ON at 4° C. After blocking (>200 ul PBS/10% FBS, 1 hr 37° C.) plates were further incubated with sample sera appropriately diluted ON at 4° C. After extensive washes, PBS/5% FBS containing biotinylated ovalbumin (10 ug/ml final,) was added to each well and plates were incubated 2 hrs at RT. Ova-specific IgE were revealed by adding streptavidin-HRP (1:5000, Pharmingen, 45 min RT) and the specific substrate. Finally, 450 nm absorbance was recorded after the reaction was stopped.

Histology and Immunohistochemistry

Bladders were explanted from the animals, longitudinally divided in two moieties, one half was either immediately fixed in formalin (10% buffered) for at least 3 hrs or snap frozen in liquid nitrogen upon inclusion in OCT freezing medium (Tissue-Tek Sakura). For histological analysis, fixed bladders were further processed and finally paraffin embedded. Five um sections were serially cut then stained with GIEMSA (BDH, 20% solution, 3 hrs RT) and then de-stained in 0.1% acetic acid for 10 sec. After a clarification step in Xylene, slides were permanently mounted with Eukitt medium and analyzed by a pathologist in a blinded fashion in order to evaluate for inflammatory cell infiltrate, mast cell numbers, and the presence of interstitial edema. A semi-quantitative histological score was used, assigning: 1 for mild infiltrate, no edema; 2 for intermediate infiltration, little edema; and 3 for severe infiltration and edema.

In selected bladder tissues, the expression of vitamin D3 receptor (VDR) was checked by performing immunohistochemical staining on frozen sections. Sections were fixed in acetone 10 min at RT. After 5 PBS washes, slides are incubated 30 min RT with methanol containing 0.3% H₂O₂, in order to quench endogenous peroxidase and then pre-incubated 1 hr with 5% normal rabbit serum. Anti VDR antibody (Affinity Bio Reagents clone 9A7 isotype IgG2b) was then added to slides (5 ug/ml final) and incubation was prolonged ON at 4° C.

After thorough washes, sections were incubated with rabbit anti rat IgG (10 ug/ml) for 1 hr at RT, washed again, and further incubated with streptavidin-HRP (vector Labs) for 30 min RT. Slides were developed by adding the specific chromogen (Vector labs) until visually good staining was achieved, counterstained with hematoxilin, and finally permanently mounted with Eukitt medium (Bio-optica).

Taq-Man Analysis

Total RNA was extracted from one half of bladder by using RNeasy Mini kit (Qiagen), treated with DNase 1 (Qiagen) and reverse transcription performed with Reverse Transcription Reagent (Applied Biosystems) with Random Hexamers (according to the manufacturer's instructions). cDNA was synthesized from 1 ug of RNA. Real-Time PCR was performed in 96-well optical reaction plates (Applied Biosystems). For each sample we amplified both the target gene and the housekeeping gene (HPRT) in different wells (singleplex) and in duplicate. The amplification Master Mix was prepared according to the following protocol (volumes refer to a single well with a final volume of 40 ul/well)): 2× TaqMan® R Universal PCR Master Mix (Applied Biosystems, 4304437): 20 ul; 20× Assay target gene: 2 ul; H₂O: 8 ul; cDNA: 10 ul. Reaction was run on an SDS 7000 (Applied Biosystems) instruments, with the following amplification program: 2′ at 50° C.; 10′ at 95° C.; 15″ at 95° C. and 1′ at 60° C. for 40 cycles; Cycle threshold (Ct) values were exported into Excel Worksheets for analysis and relative quantitations were performed using the ΔCt method. All primers used carried the FAM reporter.

Calcemia Assessment

Serum calcemia was evaluated by a commercially available colorimetric assay (Calcium Dry-Fast, Sentinel CH. Italy). Briefly, 10 ul serum were added to 100 ul reaction solution prepared according to the manufacturer's procedure in a 96-wells plate. After 5 min incubation at RT samples were read at 570 nm with a spectrophotometer and the calcium levels were calculated in triplicate by using a standard reference as directed.

Results

ELISAS

FIG. 8 shows the total amounts of IgE, and FIG. 9 shows the amounts of antigen specific IgE. The data represent a single experiment repeated at least three time, with 8-10 subject animals per group (treatment, control and serum levels pre-challenge). Results for only one dose of 75 ug/kg are shown, similar results were obtained with a dose of 30 ug/kg (not shown).

The data indicate that the procedure has been very effective in inducing an immune response, providing a 8-fold increase of antigen specific serum IgE in ovalbumin/alum treated animals compared to pre-challenged sera of the same animals. However, no significant changes were detected in the levels of either the total amount of IgE or ovalbumin specific IgE between vehicle and Compound B treated animals. This finding is as would be expected.

Serum levels of mast-cell derived chymase MMCP1 protein are shown in FIG. 10. Upon exposure to the antigen, the bladder mucosa reacts by triggering degranulation of resident mast cells and causing the release of a variety of inflammatory mediators. The serum levels of chymase MMCP1 protein are significantly lower (p<0.05) in mice treated with Compound B (75 ug/kg) than those treated with the vehicle, suggesting an inhibitory effect on mast cell induced inflammatory responses.

Calcemia

Treatment with Compound B (75 ug/kg) did not cause calcemic levels to rise above the toxicity threshold, as shown in FIG. 11 (the dotted line indicates the toxicity threshold of 10.7 mg/dl in mice). A small, but statistically significant increase in serum calcium level is noted in the Compound B treated group (p<0.05). No significant changes in serum calcium levels was detected in mice treated with a lower dosage (30 mg/kg) of Compound B (not shown)

Body Weight

FIG. 12 illustrates the variation in body weight of treated (compound B, 75 ug/kg) and control animals. Data are represented as mean values with standard deviation values shown. 8-10 subject animals per group. Data points for the two groups show no significant difference. As body weight is a good indicator of toxicity, this finding is supportive of a lack of adverse effects resulting from treatment with Compound B.

Taq-Man Analysis

The levels of various inflammatory markers are shown in FIG. 13: IL-13, MCPT2 and FcεR1α are presented for saline challenged (vehicle treated) and ovalbumin challenged (vehicle, Compound B 30 ug/kg and Compound B 75 ug/kg treated). Results were obtained by pooling equal amounts of serum recovered from test subjects. Oral treatment with Compound B significantly reduces the expression of all three inflammatory markers at both dosage levels (in a dose dependent fashion). Challenge with saline does not show any increase in the Th2/mast-cell specific markers.

FIG. 14 illustrates data on the presence of the inflammatory markers, IL-13, MMCP4 and FcεR1α for ovalbumin challenged (vehicle and Compound B treated 75 ug/kg) mice. The data is presented showing the individual values from single animals. Up-regulation of the markers is observed to varying degrees within the control group, suggesting that the animals may recover from challenge at different rates. However, oral treatment with the vitamin D₃ analogue down-modulates expression of the inflammatory markers, this finding suggests that the compounds of the invention may modulate Th2 type inflammatory responses in the bladder.

Histology

Data from the blind histological analysis of bladder sections from both vehicle and Compound B treated (75 ug/kg) animals are presented in FIG. 15. A significant reduction in mast cells (p<0.05), eosinophils (p<0.01) and lymphocytes (p<0.01) is observed, with edema appearing to be completely resolved (p<0.001).

The effects of treatment with Compound B are also apparent in the representative slides shown in FIG. 16, where no signs of edema are visible in the Compound B treated animal (75 ug/kg), but a number of lesions indicated by arrows) are visible in the control animal (vehicle treated).

Discussion

The data presented in this Example support a hypothesis that the vitamin D₃ analogue Compound B is effective in modulating the Th2-type inflammatory response in the bladder by down-modulating the expression of typical markers such as IL-13 or FcεR1α. Additionally, a diminished inflammatory cell infiltrate is also detectable within the ovalbumin-challenged bladders upon drug treatment, indicating that migration of inflammatory cells might be reduced, possibly because of a reduced cytokine/chemokine environment and an impairment of cell maturation in bone marrow. Compound B treatment also inhibits the release of MMCP1 protein in the serum, suggesting a direct effect on mast cell activation. However, it is still unclear whether this observed effect is generated by a lower number of mast cell migrating into the bladder mucosa or by a direct effect of vitamin D₃ analogues on mast cell de-granulation.

It can be concluded that oral treatment with vitamin D₃ analogue Compound B exerts anti-inflammatory and effects on the bladder in the allergen induced model of chronic bladder inflammation suggesting that vitamin D compounds represent a new therapeutic option for interstitial cystitis.

### Biological Example 5

### Evaluation of Vitamin D3 Analogues (Compounds C-I) in an In Vivo Model—Allergic IC in Mice

Method

Experiments in Example 7 were performed according to the general procedures described previously in Example 6. Mice were challenged with saline (untreated) or ovalbumin (treatment with vehicle or one of Compounds C-I at a dosage indicated in Table 6 in ug/kg).

TABLE 6
Chemical names and dosages of Compounds
A-I used in Example 7.

| Compound | Chemical Name | Dosage (ug/kg) |
|---|---|---|
| Compound C | 1,3-Di-O-acetyl-1,25- | 3 |
| | dihydroxy-16-ene-19-nor- | |
| | cholecalciferol | |
| Compound A | 1,3-Di-O-acetyl-1,25- | 0.1 |
| | dihydroxy-16,23Z-diene- | |
| | 26,27-hexafluoro-19-nor- | |
| | cholecalciferol | |
| Compound E | 1,3-Di-O-acetyl-1,25- | 0.3 |
| | dihydroxy-20-cyclopropyl- | |
| | 23E-ene-26,27-hexafluoro- | |
| | 19-nor-cholecalciferol | |
| Compound F | 1,3-Di-O-acetyl-1,25- | 30 |
| | dihydroxy-20-cyclopropyl- | |
| | cholecalciferol | |
| Compound G | A vitamin D3 analogue | 100 |
| Compound H | 1,25-Dihydroxy-16-ene-20- | 1 |
| | cyclopropyl-cholecalciferol | |
| Compound I | 1,25-dihydroxy-21-(3- | 3 |
| | hydroxy-3-methylbutyl)-19- | |
| | nor-cholecalciferol | |

Results

Experimental results for Example 7 are summarised in FIG. 17. The data is illustrated graphically in FIGS. 18 to 26.

Expression of the FcεR¹α inflammatory marker is shown in FIG. 18. Treatment with Compound E (p<0.05), Compound H (p<0.05) and in particular Compound F (p<0.001) led to statistically significant reductions in the mRNA expression level of the FcεR1α gene.

Expression of the IL-13 inflammatory marker is shown in FIG. 19. Treatment with Compound H (p<0.05) and Compound I (p<0.05), and in particular Compound E (p<0.001) and Compound F (p<0.001) led to statistically significant reductions in the mRNA expression level of the IL-13 gene.

Expression of the MMCP4 inflammatory marker is shown in FIG. 20. Treatment with Compound H (p<0.05) and Compound I (p<0.05), and in particular Compound E (p<0.001) and Compound F (p<0.001) led to statistically significant reductions in the mRNA expression level of MMCP4 gene.

FIG. 21 shows serum levels of MMCP1. Treatment with Compound F (p<0.05) and in particular Compound A (p<0.001) and Compound E (p<0.001) led to a statistically significant reduction in the serum level of the MMCP1 protein.

Data from the blind histological analysis is illustrated in FIGS. 22 to 25. Note that data for treatment with some of the tested compounds is not available at this time. Treatment with the vitamin D₃ analogue Compound C results in significantly reduced numbers of mast cells in the bladder wall (p<0.05) relative to the control (vehicle treated) animals, as shown in FIG. 22. Treatment with Compound A results in significantly reduced numbers of eosinophils in the bladder wall (p<0.05), as shown in FIG. 23. FIG. 24 shows that treatment with Compound E and Compound I both led to a significant reduction in the number of LMPC in the bladder wall (p<0.05 for both treatments. EDEMA evaluation is illustrated in FIG. 25.

Treatment with Compound C, Compound E, Compound F, Compound H and Compound I led to slight elevation of serum calcium levels relative to the vehicle treated control group (p<0.001 in all cases), with the degree of serum calcium elevation varying among treatment groups. Treatment with Compound A or Compound G showed no significant increase in serum calcium levels.

Summarizing all the results presented above in Example 7, we can conclude that these compounds can be ranked with respect to their efficacy in reducing several parameters measuring inflammation of the bladder in the allergen-induced chronic bladder inflammation model of interstitial cystitis. Efficacy ranking is as follows: Compound E>Compound F>Compound B>Compound H>Compound I>Compound A>Compound A=Compound G. Taken together, these data confirm that vitamin D compounds represent a new therapeutic option for interstitial cystitis.

### FORMULATION EXAMPLES

### Formulation Example 1

### Soft Gelatin Capsule Formulation I

| Item | Ingredients | mg/Capsule |
|---|---|---|
| 1 | Compound A | 10.001-0.02 |
| 2 | Butylated Hydroxytoluene (BHT) | 0.016 |
| 3 | Butylated Hydroxyanisole (BHA) | 0.016 |
| 4 | Miglyol 812 qs. | 160.0 |

Manufacturing Procedure:

1. BHT and BHA is suspended in Miglyol 812 and warmed to about 50° C. with stirring, until dissolved.

2. Compound A is dissolved in the solution from step 1 at 50° C.

3. The solution from Step 2 is cooled at room temperature.

4. The solution from Step 3 is filled into soft gelatin capsules.

Note: All manufacturing steps are performed under a nitrogen atmosphere and protected from light.

### Formulation Example 1B

### Soft Gelatin Capsule Formulation II

| Item | Ingredients | mg/Capsule |
|---|---|---|
| 1 | Compound A | 10.001-0.02 |
| 2 | di-.alpha.-Tocopherol | 0.016 |
| 3 | Miglyol 812 qs. | 160.0 |

Manufacturing Procedure:

1. Di-alpha-Tocopherol is suspended in Miglyol 812 and warmed to about 50° C. with stirring, until dissolved.

2. Compound A is dissolved in the solution from step 1 at 50° C.

3. The solution from Step 2 is cooled at room temperature.

4. The solution from Step 3 is filled into soft gelatin capsules.

### Formulation Example 2A

### Oral Dosage Form Soft Gelatin Capsule

A capsule for oral administration is formulated under nitrogen in amber light from 0.01 to 25.0 mg of Compound B in 150 mg of fractionated coconut oil (e.g. Miglyol 812), with 0.015 mg butylated hydroxytoluene (BHT) and 0.015 mg butylated hydroxyanisole (BHA), filled in a soft gelatin capsule.

The capsule is prepared by the following process:

1. BHT and BHA are suspended in fractionated coconut oil (e.g. Miglyol 812) and warmed to around 50° C. with stirring, until dissolved.

2. Compound B is dissolved in the solution from step 1 at 50° C.

3. The solution from step 2 is cooled to room temperature.

4. The solution from step 3 is filled into soft gelatin capsules.

All manufacturing steps are performed under a nitrogen atmosphere and protected from natural light.

### Formulation Example 2B

### Oral Dosage Form Soft Gelatin Capsule

A capsule for oral administration is formulated under nitrogen in amber light: 150 μg of Compound B in 150 mg of fractionated coconut oil (Miglyol 812), with 0.015 mg butylated hydroxytoluene (BHT) and 0.015 mg butylated hydroxyanisole (BHA), filled in a soft gelatin capsule.

### Formulation Example 2C

### Oral Dosage Form Soft Gelatin Capsule

A capsule for oral administration is formulated under nitrogen in amber light: 75 μg of Compound B in 150 mg of fractionated coconut oil (Miglyol 812), with 0.015 mg butylated hydroxytoluene (BHT) and 0.015 mg butylated hydroxyanisole (BHA), filled in a soft gelatin capsule.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended with be encompassed by the following claims.

1. (canceled)

2. (canceled)

3. A method for preventing and/or treating interstitial cystitis in a subject comprising administering to a subject in need thereof therapeutically effective amount of a vitamin D compound, thereby preventing and/or treating interstitial cystitis in said subject.

4. The method of claim 3, wherein said interstitial cystitis is characterized by the presence of symptoms of bladder dysfunction and bladder inflammation.

5. The method of claim 3 wherein the vitamin D compound is administered separately, sequentially or simultaneously in separate or combined pharmaceutical formulations with a second medicament for the treatment of interstitial cystitis.

6. (canceled)

7. A pharmaceutical formulation comprising a vitamin D compound and a pharmaceutically acceptable carrier packaged with instructions for use in the prevention and/or treatment of interstitial cystitis.

8. (canceled)

9. A kit comprising a vitamin D compound together with instructions directing administration of said compound to a subject in need of treatment and/or prevention of interstitial cystitis thereby to treat and/or prevent interstitial cystitis in said patient.

10. The method of claim 3, wherein said vitamin D compound is a compound of the formula: 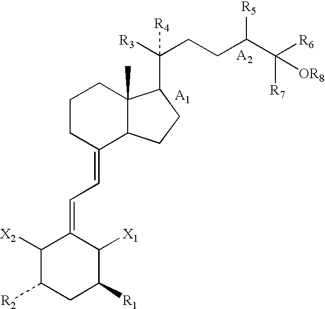
wherein:
A₁ is single or double bond;
A₂ is a single, double or triple bond;
X₁ and X₂ are each independently H or ═CH₂, provided X₁ and X₂ are not both ═CH₂;
R₁ and R₂ are each independently OC(O)C₁-C₄ alkyl, OC(O)hydroxyalkyl, OROC(O)haloalkyl, OAc;
R₃, R₄ and R₅ are each independently hydrogen, C₁-C₄ alkyl, hydroxyalkyl, or haloalkyl, or R₃ and R₄ taken together with C₂₀ form C₃-C₆ cycloalkyl; and
R₆ and R₇ are each independently C₁₋₄alkyl or haloalkyl; and
R₈ is H, —COC₁-C₄alkyl, —COhydroxyalkyl or —COhaloalkyl; and
pharmaceutically acceptable esters, salts, and prodrugs thereof.

11. The method of claim 3, wherein said vitamin D compound is a compound of the formula: 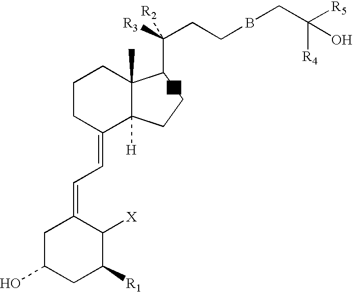
wherein:
X is H₂ or CH₂;
R₁ is hydrogen, hydroxy or fluorine;
R₂ is hydrogen or methyl;
R₃ is hydrogen or methyl provided that when R₂ or R₃ is methyl, R₃ or R₂ must be hydrogen;
R₄ is methyl, ethyl or trifluoromethyl;
R₅ is methyl, ethyl or trifluoromethyl;
A is a single or double bond; and
B is a single, E-double, Z-double or triple bond.

12. The method according to claim 11, wherein each of R₄ and R₅ is methyl or ethyl.

13. The method of claim 10, wherein said vitamin D compound is 1,3-Di-O-acetyl-1,25-dihydroxy-16,23Z-diene-26,27-hexafluoro-19-nor-cholecalciferol, having the formula:

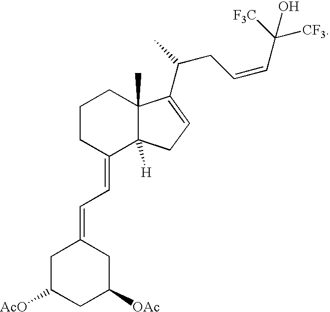

14. The method of claim 11, wherein said vitamin D compound is 1-alpha-fluoro-25-hydroxy-16,23E-diene-26,27-bishomo-20-epi-cholecalciferol, having the formula:

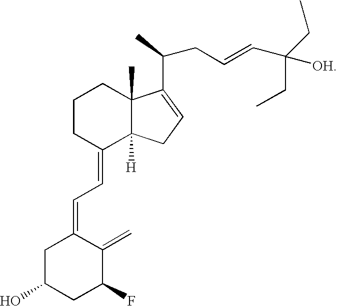

15. The method of claim 10, wherein said compound is 1,3-Di-O-acetyl-1,25-dihydroxy-20-cyclopropyl-23E-ene-26,27-hexafluoro-19-nor-cholecalciferol, having the formula:

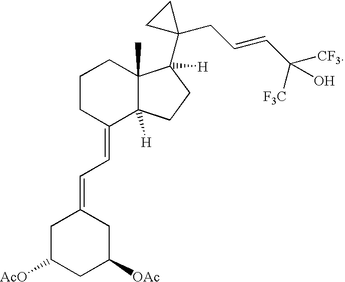

16. The method of claim 3, wherein said vitamin D compound is administered as a pharmaceutical composition comprising a therapeutically effective amount of the vitamin D compound and a pharmaceutically acceptable diluent or carrier therefore.

17. The method of claim 3, wherein the subject is a mammal.

18. The method of claim 17, wherein the mammal is a human.

19. The pharmaceutical formulation of claim 7, wherein the instructions for use are in accordance with the method of claim 3.

20. The pharmaceutical formulation of claim 9, wherein the instructions are in accordance with the method of claim 3.
